(19) United States
(12) Patent Application Publication
(10) Pub. No.: US 2021/0387954 A1
(43) Pub. Date: Dec. 16, 2021

(54) OXADIAZOLES AS FUNGICIDES

(71) Applicant: PI INDUSTRIES LIMITED, Rajasthan (IN)

(72) Inventors: Paras Raybhan BHUJADE, Maharashtra (IN); Maruti N NAIK, Karnataka (IN); Visannagari RAMAKRISHNA, Telangana (IN); J. YUVARAJ, Tamil Nadu (IN); Suresh KUMBAR, Karnataka (IN); Sachin Nagnath GUMME, Maharashtra (IN); Santosh Shridhar AUTKAR, Maharashtra (IN); Ruchi GARG, Varanasi-UP (IN); Hagalavadi M VENKATESHA, Karnataka (IN); Alexander G.M. KLAUSENER, Pulheim (IN); Nilesh Bharat ADHAV, Maharashtra (IN)

(73) Assignee: PI INDUSTRIES LIMITED, Rajasthan (IN)

(21) Appl. No.: 17/250,939

(22) PCT Filed: Sep. 30, 2019
(86) PCT No.: PCT/IB2019/058277

§ 371 Date: Mar. 31, 2021

(30) Foreign Application Priority Data
Oct. 1, 2018 (IN) 201811037145

(51) Int. Cl. C07D 271/06; A01N 43/82

(52) U.S. Cl. CPC C07D 271/06; A01N 43/82

(57) ABSTRACT

The present invention relates to novel oxadiazoles of Formula I.

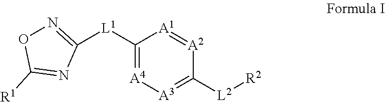

wherein, R¹, L¹, A¹, A², A³, A⁴, L² and R² are as defined in the detailed description. The present invention also relates to a combination or a composition comprising the compound of Formula I.

### FIELD OF THE INVENTION

The present invention relates to novel oxadiazoles, their N-oxides, metal complexes, isomers, polymorphs and/or the agriculturally acceptable salts thereof and to a process for preparing the same.

Further, the present invention relates to a combination and to compositions comprising novel oxadiazoles of the present invention. Still further, the present invention relates to the use of novel oxadiazoles for controlling or preventing phytopathogenic fungi and to a method for controlling or preventing harmful phytopathogenic fungi.

### BACKGROUND

Oxadiazoles have already been disclosed in the literature. For example in JP56065881, JP63162680, JPS6061573, JPS6296480, JPS6051188, JP2005336101, WO2005051932, EP3165093, EP3165094, EP3167716, EP3165093, JP2017190296, U.S. Pat. No. 4,488,897, WO2015185485, WO2017055469, WO2017055473, WO2017076739, WO2017076740, WO2017081311, WO2017085098, WO2017085100, WO2017093019, WO2017093348, WO2017102006, WO2017103219, WO2017103223, WO2017109044, WO2017110861, WO2017110862, WO2017110863, WO2017110864, WO2017110865, WO2017111152, WO2017118689, WO2017148797, WO2017157962, WO2017162868, WO2017169893, WO2017174158, WO2017178245, WO2017178549, WO2017198852, WO2017207757, WO2017211649, WO2017211650, WO2017211652, WO2017213252, WO2017220485, WO201772247, WO201776742, WO201776757, WO201776935, WO201781309, WO201781310, WO201781311, WO201781312, WO2018015447, WO2018015449, WO2018015458, WO2018056340, WO2018055135, WO2018080859, WO2018118781, WO2018117034, WO2018153730 and WO2018114393, various oxadiazoles have been disclosed.

The oxadiazoles reported in the above literature have disadvantages in certain aspects, such as that they exhibit a narrow spectrum of application, or they do not have satisfactory fungicidal activity, particularly at low application rates.

Therefore, it is an object of the present invention to provide compounds having an improved/enhanced activity and/or a broader activity spectrum against phytopathogenic fungi.

This objective is achieved by the use of novel oxadiazoles of the present invention for controlling or preventing phytopathogenic fungi.

### SUMMARY

The present invention relates to novel oxadiazoles of Formula I.

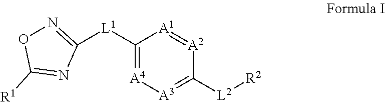

wherein, R¹, L¹, A¹, A², A³, A⁴, L² and R² are as defined in the detailed description.

The compound of Formula I have now been found to have advantages over the compounds reported in the literature in either of improved fungicidal activity, broader spectrum biological activity, lower application rates, biological or environmental properties, and/or enhanced plant compatibility.

More specifically, the present invention further relates to combinations comprising novel oxadiazoles and at least one further pesticidally active substance for controlling or preventing phytopathogenic fungi which are difficult to control or prevent.

The present invention still further relates to compositions comprising novel oxadiazoles or novel oxadiazoles in combination with further pesticidally active substances.

The present invention still further relates to a method and use of novel oxadiazoles, of combinations or of compositions thereof for controlling and or preventing plant diseases, particularly phytopathogenic fungi.

### DETAILED DESCRIPTION

### Definitions

The definitions provided herein for the terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes, helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” refers to a living organism of the Phylum Nematoda. The term “helminths” includes roundworms, heartworms, phytophagous nematodes (Nematoda), flukes (Tematoda), acanthocephala and tapeworms (Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food, feed and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

Compounds of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated.

The term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Non-limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxy, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Non-limiting examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

Non-limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “alkynyl” can also include moieties comprised of multiple triple bonds such as 2,5-hexadiynyl.

The term “cycloalkyl” means alkyl closed to form a ring. Non-limiting examples include cyclopropyl, cyclopentyl and cyclohexyl. This definition also applies to cycloalkyl as a part of a composite substituent, for example cycloalkylalkyl etc., unless specifically defined elsewhere.

The term “cycloalkenyl” means alkenyl closed to form a ring including monocyclic, partially unsaturated hydrocarbyl groups. Non-limiting examples include cyclopropenyl, cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. Non-limiting examples include cyclopropynyl, cyclopentynyl and cyclohexynyl.

This definition also applies to cycloalkynyl as a part of a composite substituent, for example cycloalkynylalkyl etc., unless specifically defined elsewhere.

The term “cycloalkoxy”, “cycloalkenyloxy” and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl”, “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non-limiting examples of “haloalkylsulfinyl” include CF₃S(O), CCl₃S(O), CF₃CH₂S(O) and CF₃CF₂S(O). Non-limiting examples of “haloalkylsulfonyl” include CF₃S(O)₂, CCl₃S(O)₂, CF₃CH₂S(O)₂ and CF₃CF₂S(O)₂.

The term “hydroxy” means —OH, Amino means —NRR, wherein R can be H or any possible substituent such as alkyl. Carbonyl means —C(═O)—, carbonyloxy means —OC(═O)—, sulfinyl means SO, sulfonyl means S(O)₂.

The term “alkoxy” used either alone or in compound words included C₁ to C₂₄ alkoxy, preferably C₁ to C₁₅ alkoxy, more preferably C₁ to C₁₀ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “alkoxyalkyl” denotes alkoxy substitution on alkyl. Non-limiting examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkoxyalkoxy” denotes alkoxy substitution on alkoxy.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

The term “alkylthioalkyl” denotes alkylthio substitution on alkyl. Non-limiting examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. The term “cycloalkylalkylamino” denotes cycloalkyl substitution on alkyl amino.

The terms “alkoxyalkoxyalkyl”, “alkylaminoalkyl”, “dialkylaminoalkyl”, “cycloalkylaminoalkyl”, “cycloalkylaminocarbonyl” and the like, are defined analogously to “alkylthioalkyl” or “cycloalkylalkylamino”.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” denotes alkoxy carbonyl substitution on alkyl amino. “Alkylcarbonylalkylamino” denotes alkyl carbonyl substitution on alkyl amino. The terms alkylthioalkoxycarbonyl, cycloalkylalkylaminoalkyl and the like are defined analogously.

Non-limiting examples of “alkylsulfinyl” include methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl and the different isomers. The term “arylsulfinyl” includes Ar—S(O), wherein Ar can be any carbocyle or heterocylcle. This definition also applies to alkylsulphinyl as a part of a composite substituent, for example haloalkylsulphinyl etc., unless specifically defined elsewhere.

Non-limiting examples of “alkylsulfonyl” include methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl and the different isomers. The term “arylsulfonyl” includes Ar—S(O)₂, wherein Ar can be any carbocyle or heterocylcle. This definition also applies to alkylsulphonyl as a part of a composite substituent, for example alkylsulphonylalkyl etc., unless defined elsewhere.

“Alkylamino”, “dialkylamino”, and the like, are defined analogously to the above examples.

The term “carbocycle or carbocyclic” includes “aromatic carbocyclic ring system” and “non-aromatic carbocylic ring system” or polycyclic or bicyclic (spiro, fused, bridged, nonfused) ring compounds in which ring may be aromatic or non-aromatic (where aromatic indicates that the Huckel rule is satisfied and non-aromatic indicates that the Huckel rule is not satisfied).

The term “heterocycle or heterocyclic” includes “aromatic heterocycle or heteroaryl ring system” and “non-aromatic heterocycle ring system” or polycyclic or bicyclic (spiro, fused, bridged, nonfused) ring compounds in which ring may be aromatic or non-aromatic, wherein the heterocycle ring contains at least one heteroatom selected from N, O, S(O)₀₋₂, and or C ring member of the heterocycle may be replaced by C(═O), C(═S), C(═CR*R*) and C═NR*, * indicates integers.

The term “non-aromatic heterocycle” or “non-aromatic heterocyclic” means three- to fifteen-membered, preferably three- to twelve-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; non-limiting examples oxetanyl, oxiranyl, aziridinyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines, 2,3,4,5-tetrahydro[1H]azepin-1- or -2- or -3- or -4- or -5- or -6- or -7-yl, 3,4,5,6-tetra-hydro[2H]azepin-2- or -3- or -4- or -5- or -6- or -7-yl, 2,3,4,7-tetrahydro[1H]azepin-1- or -2- or -3- or -4- or -5- or -6- or -7-yl, 2,3,6,7-tetrahydro[1H]azepin-1- or -2- or -3- or -4- or -5- or -6- or -7-yl, hexahydroazepin-1- or -2- or -3- or -4-yl, tetra- and hexahydrooxepinyl such as 2,3,4,5-tetrahydro[1H]oxepin-2- or -3- or -4- or -5- or -6- or -7-yl, 2,3,4,7-tetrahydro[1H]oxepin-2- or -3- or -4- or -5- or -6- or -7-yl, 2,3,6,7-tetrahydro[1H]oxepin-2- or -3- or -4- or -5- or -6- or -7-yl, hexahydroazepin-1- or -2- or -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl.

This definition also applies to heterocyclyl as a part of a composite substituent, for example heterocyclylalkyl etc., unless specifically defined elsewhere.

The term “heteroaryl” or “aromatic heterocyclic” means 5 or 6-membered, fully unsaturated monocyclic ring system containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom; 5-membered heteroaryl groups which, in addition to carbon atoms, may contain one to four nitrogen atoms or one to three nitrogen atoms and one sulphur or oxygen atom as ring members, non-limiting examples furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, non-limiting examples 1-pyrrolyl, 1-pyrazolyl, 1,2,4-triazol-1-yl, 1-imidazolyl, 1,2,3-triazol-1-yl and 1,3,4-triazol-1-yl. 6-membered heteroaryl which contains one to four nitrogen atoms: 6-membered heteroaryl groups which, in addition to carbon atoms, may contain, respectively, one to three and one to four nitrogen atoms as ring members, non-limiting examples 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: non-limiting examples indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: non-limiting examples quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

The term “trialkylsilyl” includes 3 branched and/or straight-chain alkyl radicals attached to and linked through a silicon atom such as trimethylsilyl, triethylsilyl and t-butyl-dimethylsilyl. “Halotrialkylsilyl” denotes at least one of the three alkyl radicals is partially or fully substituted with halogen atoms which may be the same or different. The term“alkoxytrialkylsilyl” denotes at least one of the three alkyl radicals is substituted with one or more alkoxy radicals which may be the same or different. The term “trialkylsilyloxy” denotes a trialkylsilyl moiety attached through oxygen.

Non-limiting examples of “alkylcarbonyl” include C(═O)CH₃, C(═O)CH₂CH₂CH₃ and C(═O)CH(CH₃)₂. Non-limiting examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Non-limiting examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Non-limiting examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH₃)C(═O). Non-limiting examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O). Non-limiting examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylalkyl amino and the like are defined analogously Non-limiting examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O).

The term “amide” means A-R′C═ONR″—B, wherein R′ and R″ indicates substituents and A and B indicate any group.

The term “thioamide” means A-R′C═SNR″—B, wherein R′ and R″ indicates substituents and A and B indicate any group.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of Formula I is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

When a group contains a substituent which can be hydrogen, then, when this substituent is taken as hydrogen, it is recognized that said group is being un-substituted.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the description/claims though might form a critical part of the present invention of the present invention, any deviation from such numerical values shall still fall within the scope of the present invention if that deviation follows the same scientific principle as that of the present invention disclosed in the present invention.

The inventive compound of the present invention may, if appropriate, be present as mixtures of different possible isomeric forms, especially of stereoisomers, for example E and Z, threo and erythro, and also optical isomers, but if appropriate also of tautomers. Both the E and the Z isomers, and also the threo and erythro isomers, and the optical isomers, any desired mixtures of these isomers and the possible tautomeric forms are disclosed and claimed.

The term “pest” for the purpose of the present disclosure includes but is not limited to fungi, stramenopiles (oomycetes), bacteria, nematodes, mites, ticks, insects and rodents.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present disclosure the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts; coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plant for the purpose of the present invention includes but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for use of human and animals.

The term “plant parts” is understood to mean all parts and organs of plants above and below the ground. For the purpose of the present disclosure the term plant parts includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxillary buds, meristems, nodes and internodes.

The term “locus thereof” includes soil, surroundings of plant or plant parts and equipment or tools used before, during or after sowing/planting a plant or a plant part.

Application of the compounds of the present disclosure or the compound of the present disclosure in a composition optionally comprising other compatible compounds to a plant or a plant material or locus thereof include application by a technique known to a person skilled in the art which include but is not limited to spraying, coating, dipping, fumigating, impregnating, injecting and dusting.

The term “applied” means adhered to a plant or plant part either physically or chemically including impregnation.

Accordingly, novel oxadiazoles according to the present invention are represented by a compound of Formula I and include N-oxides, metal complexes, isomers, polymorphs or the agriculturally acceptable salts thereof.

The present invention relates to a compound of Formula I,

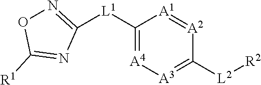

wherein,

R¹ is selected from the group consisting of C₁-C₂-monohaloalkyl, C₁-C₂-dihaloalkyl, C₁-C₂-trihaloalkyl, C₁-C₂-tetrahaloalkyl, and C₁-C₂-pentahaloalkyl;

A¹ is CR^{A1} or N;

A² is CR^{A2} or N;

A³ is CR^{A3} or N; &

A⁴ is CR^{A4} or N; wherein no more than two of A¹, A², A³ & A⁴ are nitrogen;
wherein, R^{A1}, R^{A2}, R^{A3}, R^{A4}, and R^{A5} are independently and optionally selected from the group consisting of hydrogen, halogen, cyano, nitro, amino, hydroxy, C₁-C₆-alkyl, C₃-C₆-cycloalkyl, C₁-C₆-haloalkyl, C₁-C₆-hydroxyalkyl, C₁-C₆-alkoxy, C₁-C₆-alkoxy-C₁-C₆-alkyl, and C₁-C₆-haloalkoxy;

either R^{A1} and R^{A2} or R^{A3} and R^{A4} or both R^{A1} and R^{A2} as well as R^{A3} and R^{A4} together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S(O)₀₋₂; wherein, the carbocyclic or the heterocyclic ring or ring system may optionally further be substituted with one or more of halogen, C₁-C₆-alkyl, C₃-C₆-cycloalkyl, C₁-C₆-haloalkyl, C₁-C₆-hydroxyalkyl, C₁-C₆-alkoxy and C₁-C₆-haloalkoxy; L¹ is —C(R⁴R⁵)— or —C(═W)—; L² is a direct bond, —C(R^{4a}R^{5a})—, —(NR⁶)₀₋₁C(═W¹)—(NR⁶)₀₋₁, —C(F₂)—, —C(R^{4a}R^{5a})C(═O)—, —O—, —(CR^{4a}R^{5a})₀₋₂S(═O)₀₋₂—, —N(R⁶)—, —(CR^{4a}R^{5a})₀₋₂C(═W¹)NR⁶(CR^{4a}R^{5a})₀₋₂—, and —NR⁶S(═O)₀₋₂—;
wherein W and W¹ is O or S;

wherein, R² is selected from the group consisting of hydrogen, halogen, cyano, nitro, amino, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, aryloxy, heteroaryloxy, C₃-C₈-heterocylyloxy, C₃-C₈-cycloalkyloxy, C₁-C₆-haloalkoxy, C₁-C₆-haloalkoxycarbonyl, C₁-C₆-alkylthio, arylthio, heteroarylthio, C₄-C₅-heterocyclylthio, C₁-C₆-haloalkylthio, C₁-C₆-haloalkylsulfinyl, C₁-C₆-haloalkylsulfonyl, arylsulfonyl, heteroarylsulfonyl, C₃-C₈-cycloalkylsulfonyl C₁-C₆-alkylsulfinyl, C₁-C₆-alkylsulfonyl, C₁-C₆-alkylamino, arylamino, heteroarylamino, C₄-C₈-heterocyclylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₃-C₆-cycloalkoxycarbonyl, aryloxycarbonyl, heteroaryloxycarbonyl, heterocycloxycarbonyl, C₁-C₆-alkylaminocarbonyl, C₁-C₆-dialkylaminocarbonyl, arylaminocarbonyl, heteroarylaminocarbonyl, C₃-C₆-cycloalkylaminocarbonyl, C₁-C₆-alkylaminocarbonylamino, C₁-C₆-dialkylaminocarbonylamino, arylaminocarbonylamino, heteroarylaminocarbonylamino, C₃-C₆-cycloalkylaminocarbonylamino, C₁-C₆-alkylcarbonylamino, C₃-C₆-cycloalkylcarbonylamino, arylcarbonylamino, heteroarylcarbonylamino, heterocyclylcarbonylamino, C₁₋C₆-haloalkylcarbonylamino, C₁-C₆-alkyloxycarbonylamino, aryloxycarbonylamino, heterocycloxycarbonylamino, heteroaryloxycarbonylamino, C₃-C₆-cycloalkyloxycarbonylamino, C₁-C₆-alkoxycarbonyloxy, C₁-C₆-alkylaminocarbonyloxy, or C₁-C₆-dialkylaminocarbonyloxy, sulfilimines, sulfoximines, sulfonamide, and sulfinamide; R² may optionally further be substituted with one or more R⁷; or

R² is phenyl, benzyl, naphthyl, a 5- or 6-membered aromatic ring, an 8- to 11-membered aromatic multi-cyclic ring system, an 8- to 11-membered aromatic fused ring system, a 5- or 6-membered heteroaromatic ring, an 8- to 11-membered heteroaromatic multi-cyclic ring system or an 8- to 11-membered heteroaromatic fused ring system; wherein the heteroatom of the heteroaromatic ring or ring system is one or more heteroatom selected from N, O or S, and each phenyl, benzyl, aromatic or heteroaromatic ring or ring system may be optionally substituted with one or more substituents selected from R³; or

or R² and R⁶ together with the atoms to which they are attached form a 4-, 5-, 6- or 7-membered nonaromatic heterocyclic ring, an 8- to 15-membered nonaromatic hetero-multicyclic ring system, an 5- to 15 membered hetero-spirocyclic ring system, or an 8- to 15-membered nonaromatic heterocyclic fused ring system, wherein the heteroatom of the nonaromatic heterocyclic ring or ring system is selected from N, O or S(O)₀₋₂; and the C ring member of the nonaromatic heterocyclic ring or ring system may be replaced with C(═O), C(═S), C(═CR^{4a}R^{5b}) or C(═NR^{6a}) and each or nonaromatic heterocyclic ring or ring system may be optionally substituted with one or more substituents selected from R³;
wherein, R³ is independently selected from halogen, cyano, nitro, hydroxy, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₃-C₈-cycloalkyl-C₁-C₆-alkyl, C₃-C₈-cycloalkyl-C₃-C₈-cycloalkyl, C₃-C₈-cycloalkenyl, C₁-C₆-alkoxy-C₁-C₆-alkyl, C₃-C₈-cycloalkoxy-C₁-C₆-alkyl, C₁-C₆-alkylsulfinyl-C₁-C₆-alkyl, C₁-C₆-alkylsulfonyl-C₁-C₆-alkyl, C₁-C₆-alkylamino, di-C₁-C₆-alkylamino, C₁-C₆-alkylamino-C₁-C₆-alkyl, di-C₁-C₆-alkylamino-C₁-C₆-alkyl, C₁-C₆-haloalkylamino-C₁-C₆-alkyl, C₃-C₈-cycloalkylamino, C₃-C₈-cycloalkylamino-C₁-C₆-alkyl, C₁—C₆-alkylcarbonyl, C₁-C₆-haloalkoxy-C₁-C₆-alkyl, C₁-C₆-hydroxyalkyl, C₂-C₆-hydroxyalkenyl, C₂-C₆-hydroxyalkynyl, C₂-C₆-alkenyloxy, C₂-C₆-haloalkenyloxy, C₂-C₆-alkynyloxy, C₁-C₆-alkylcarbonylalkoxy, C₁-C₆-alkylthio, C₁-C₆-haloalkylthio, C₃-C₈-cycloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkylsulfonyl, C₃-C₈-cycloalkylsulfinyl, C₁-C₆-alkylsulfonylamino, C₁-C₆-haloalkylsulfonylamino, C₁-C₆-alkylsulfonyloxy, C₆-C₁₀-arylsulfonyloxy, C₆-C₁₀-arylsulfonyl, C₆-C₁₀-arylsulfinyl, C₆-C₁₀-arylthio, C₁-C₆-cyanoalkyl, C₁-C₆-haloalkylamino, C₁-C₆-alkoxyamino, C₁-C₆-haloalkoxyamino, C₁-C₆-alkoxycarbonylamino, C₁-C₆-alkylcarbonyl-C₁-C₆-alkylamino, C₂-C₆-alkenylthio, di(C₁-C₆-haloalkyl)amino-C₁-C₆-alkyl, C₁-C₆-alkylaminocarbonylamino, di(C₁-C₆-haloalkyl)amino, sulfilimines, sulfoximines or SF₅; wherein, R³ may be optionally substituted with halogen, cyano, amino, C₁-C₆-alkyl, C₁-C₆-alkoxy, C₁-C₆-alkylamino-C₁-C₆-alkoxy, C₁-C₆-alkylthio, and C₃-C₈-cycloalkyl; or
R⁷ is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₃-C₈-cycloalkyloxy, aryloxy, C₁-C₆-haloalkoxy, and C₁-C₆-haloalkoxycarbonyl; or
two R⁷ together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S; or
R⁷ is phenyl, benzyl, a 5-membered aromatic ring, a 5- or 6-membered heteroaromatic ring; wherein heteroatom of the heteroaromatic ring is selected from N, O or S; or
R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N, O or S(O)₀₋₂; and the C ring member of the nonaromatic carbocyclic or nonaromatic heterocyclic ring may be replaced with C(═O), C(═S), C(═CR^{4c}R^{5c}) or C(═NR^{6b});
wherein, R⁷ may be further substituted with one or more R^{4d} on C atom and with one or more R^{6c} on N atom;
   R⁴, R^{4a}, R^{4b}, R^{4c}, R^{4d}, R⁵, R^{5a}, R^{5b}, and R^{5c} are independently selected from hydrogen, halogen, cyano, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₃-C₈-cycloalkyloxy, C₁-C₄-alkoxy, and C₁-C₄-haloalkoxy; or
   all or either of R⁴ and R⁵; R^{4a} and R^{5a}; R^{4b} and R^{5b}; and R^{4c} and R^{5c}; together with the atoms to which they are attached may form a C₃-C₆ non-aromatic carbocylic ring or C₃-C₆ non-aromatic heterocylic ring;
   R⁶, R^{6a}, R^{6b}, and R^{6c} are independently selected from the group consisting of hydrogen, cyano, hydroxy, NR^{b}R^{c}, (C═O)—R^{d}, (C═O)(C═O)—R^{d}, S(O)₀₋₂R^{e}, C₁-C₆-alkyl, C₁₋C₆-haloalkyl, C₁₋C₆-alkoxy, C₁₋C₆-haloalkoxy, C₁₋C₆-alkylamino, di-C₁₋C₆-alkylamino, tri-C₁₋C₆-alkylamino, and C₃-C₈-cycloalkyl;
      R^{b} and R^{c} represent hydrogen, hydroxyl, cyano, C₁-C₄-alkyl, C₁-C₄-haloalkyl, C₁-C₄-alkoxy, C₃-C₈-cycloalkyl, aryl, heteroaryl, C₄-C₆-heterocyclyl, and C₃-C₈-halocycloalkyl;
      R^{d} represents hydrogen, hydroxy, halogen, NR^{b}R^{c}, C₁-C₆-alkyl, C₁₋C₆-haloalkyl, C₁₋C₆-alkoxy, C₁-C₆-haloalkoxy, C₃-C₈-cycloalkyl aryloxy, heteroaryloxy, C₃-C₈-cycloalkoxy, and C₃-C₈-halocycloalkyl; and
      R^{e} represents hydrogen, halogen, cyano, C₁-C₆-alkyl, C₁-C₆-haloalkyl, C₁-C₆-alkoxy, C₁-C₆-haloalkoxy, C₃-C₈-cycloalkyl, and C₃-C₈-halocycloalkyl;
         or N-oxides, metal complexes, isomers, polymorphs or the agriculturally acceptable salts thereof.

Particularly, the compound of Formula I is as defined herein after, wherein

R¹ is C₁-C₂-dihaloalkyl or C₁-C₂-trihaloalkyl;

A¹ is CR^{A1} or N;

A² is CR^{A2} or N;

A³ is CR^{A3} or N; &

A⁴ is CR^{A4} or N; wherein no more than one of A¹, A², A³ & A⁴ are nitrogen;
wherein, R^{A1}, R^{A2}, R^{A3}, R^{A4}, and R^{A5} are independently and optionally selected from the group consisting of hydrogen, halogen, cyano, C₁-C₆-alkyl, C₃-C₆-cycloalkyl, C₁-C₆-haloalkyl, and C₁-C₆-alkoxy;

L¹ is —C(R⁴R⁵)— or —C(═W)—;

L² is —(NR⁶)₀₋₁C(═W¹)—(NR⁶)₀₋₁, —(CR^{4a}R^{5a})₁₋₂S(═O)₀₋₂—, —(CR^{4a}R^{5a})₀₋₂C(═W¹)NR⁶(CR^{4a}R^{5a})₀₋₂—, and NR⁶—NR⁶S(═O)₀₋₂—;
wherein W and W¹ is O or S;
   wherein, R² is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, aryloxy, heteroaryloxy, C₃-C₈-heterocylyloxy, C₃-C₈-cycloalkyloxy, C₁-C₆-alkylthio, arylthio, heteroarylthio, C₄-C₈-heterocyclylamino, C₁-C₆-dialkylamino, and C₃-C₈-cycloalkylamino; or

R² is phenyl, benzyl, a 5- or 6-membered heteroaromatic ring; wherein the heteroatom of the heteroaromatic ring is one or more heteroatom selected from N, O or S, and each phenyl, benzyl or heteroaromatic ring may be optionally substituted with one or more substituents selected from R³; or

or R² and R⁶ together with the atoms to which they are attached form a 4-, 5- or 6-membered nonaromatic heterocyclic ring, wherein the heteroatom of the nonaromatic heterocyclic ring is selected from N or O; and nonaromatic heterocyclic ring may be optionally substituted with one or more substituents selected from R³;
wherein, R³ is independently selected from halogen, cyano, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₁-C₆-alkylamino, di-C₁-C₆-alkylamino, and C₁-C₆-alkoxy; or
   R⁴, R^{4a}, R^{4b}, R⁵, R^{5a} and R^{5b} are independently selected from hydrogen, halogen, C₁-C₄-alkyl, C₁-C₄-haloalkyl, C₂-C₄-haloalkenyl, C₃-C₆-cycloalkyl, C₃-C₆-halocycloalkyl, C₃-C₈-cycloalkyloxy, C₁-C₄-alkoxy, and C₁-C₄-haloalkoxy; or
   all or either of R⁴ and R⁵; R^{4a} and R^{5a}; and R^{4b} and R^{5b}; together with the atoms to which they are attached may form a C₃-C₆ non-aromatic carbocylic ring or C₃-C₆ non-aromatic heterocylic ring;
   R⁶ and R^{6a} are independently selected from the group consisting of hydrogen, S(O)₀₋₂R^{e}, C₁-C₆-alkyl, C₁-C₆-haloalkyl, C₁-C₆-alkoxy, C₁-C₆-haloalkoxy, and C₃-C₈-cycloalkyl;
      R^{e} represents hydrogen, C₁-C₆-alkyl, C₁-C₆-haloalkyl, C₁-C₆-alkoxy, C₁-C₆-haloalkoxy, C₃-C₈-cycloalkyl, and C₃-C₈-halocycloalkyl;
         or N-oxides, metal complexes, isomers, polymorphs or the agriculturally acceptable salts thereof.

More particularly, the compound is as defined hereinafter, wherein

R¹ is C₁-C₂-trihaloalkyl;

A¹ is CH;

A² is CH;

A³ is CH; &

A⁴ is CH;

L¹ is —C(R⁴R⁵)— or —C(═W)—;

L² is —(NR⁶)₀₋₁C(═W¹)—(NR⁶)₀₋₁, —(CR^{4a}R^{5a})₁₋₂S(═O)₀₋₂—, —(CR^{4a}R^{5a})₀₋₂C(═W¹)NR⁶(CR^{4a}R^{5a})₀₋₂—, and NR⁶—NR⁶S(═O)₀₋₂—;
wherein W and W¹ is O or S;
   wherein, R² is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-alkoxy, aryloxy, heteroaryloxy, C₃-C₈-heterocylyloxy, C₄-C₈-heterocyclylamino, and C₁-C₆-dialkylamino; or

R² is phenyl, benzyl, a 5- or 6-membered heteroaromatic ring; wherein the heteroatom of the heteroaromatic ring is one or more heteroatom selected from N, O or S, and each phenyl, benzyl or heteroaromatic ring may be optionally substituted with one or more substituents selected from R³; or

or R² and R⁶ together with the atoms to which they are attached form a 4-, 5- or 6-membered nonaromatic heterocyclic ring, wherein the heteroatom of the nonaromatic heterocyclic ring is selected from N or O; and nonaromatic heterocyclic ring may be optionally substituted with one or more substituents selected from R³;
wherein, R³ is independently selected from halogen, cyano, C₁-C₆-alkyl, C₁-C₆-haloalkyl, C₃-C₈-cycloalkyl, C₁-C₆-alkylamino, di-C₁-C₆-alkylamino, and C₁-C₆-alkoxy; or
   R⁴, R^{4a}, R^{4b}, R⁵, R^{5a} and R^{5b} are independently selected from hydrogen, halogen, C₁-C₄-alkyl, C₁-C₄-haloalkyl, C₃-C₆-cycloalkyl, C₃-C₆-halocycloalkyl, C₃-C₈-cycloalkyloxy, C₁-C₄-alkoxy and C₁-C₄-haloalkoxy; or
   all or either of R⁴ and R⁵; R^{4a} and R^{5a}; and R^{4b} and R^{5b}; together with the atoms to which they are attached may form a C₃-C₆ non-aromatic carbocylic ring;
   R⁶ and R^{6a} are independently selected from the group consisting of hydrogen, C₁-C₆-alkyl, C₁₋C₆-haloalkyl, and C₃-C₈-cycloalkyl;
      or N-oxides, metal complexes, isomers, polymorphs or the agriculturally acceptable salts thereof.

The following compounds are excluded from the scope of the present invention:
N-[4-[[5-(trifluoromethyl)-1,2,4-oxadiazol-3-yl]methyl]phenyl]-methanesulfonamide [Cas No. 1128079-05-9],
N-[4-[[5-(trifluoromethyl)-1,2,4-oxadiazol-3-yl]methyl]phenyl]-acetamide [Cas No. 943828-64-6],
1,2,4-Oxadiazole, 3-[(2,6-dichloro-4-hydrazinylphenyl)methyl]-5-(trifluoromethyl) [Cas No. 164157-03-3], and
4-[[5-(trifluoromethyl)-1,2,4-oxadiazol-3-yl]methyl]-benzoic acid methyl ester [Cas No. 2368917-79-5].

Most particularly, the compound of Formula I is selected from the group consisting of:
4-methyl-N-(4-((5-(trifluoromethyl)-1,2,4-oxadiazol-3-yl)methyl)phenyl)benzamide; N-(4-((5-(trifluoromethyl)-1,2,4-oxadiazol-3-yl)methyl)phenyl)benzamide; N-(4-((5-(trifluoromethyl)-1,2,4-oxadiazol-3-yl)methyl)phenyl)picolinamide; N-(4-((5-(trifluoromethyl)-1,2,4-oxadiazol-3-yl)methyl)phenyl)nicotinamide; N-(4-((5-(trifluoromethyl)-1,2,4-oxadiazol-3-yl)methyl)phenyl)isonicotinamide; 2-phenyl-N-(4-((5-(trifluoromethyl)-1,2,4-oxadiazol-3-yl)methyl)phenyl)acetamide; 4-cyano-N-(4-((5-(trifluoromethyl)-1,2,4-oxadiazol-3-yl)methyl)phenyl)benzamide; 4-(trifluoromethyl)-N-(4-((5-(trifluoromethyl)-1,2,4-oxadiazol-3-yl)methyl)phenyl)benzamide; 4-fluoro-N-(4-((5-(trifluoromethyl)-1,2,4-oxadiazol-3-yl)methyl)phenyl)benzamide; 4-chloro-N-(4-((5-(trifluoromethyl)-1,2,4-oxadiazol-3-yl)methyl)phenyl)benzamide; 2-(4-fluorophenyl)-N-(4-((5-(trifluoromethyl)-1,2,4-oxadiazol-3-yl)methyl)phenyl)acetamide; N-(4-fluorobenzyl)-4-((5-(trifluoromethyl)-1,2,4-oxadiazol-3-yl)methyl)benzamide; morpholino(4-((5-(trifluoromethyl)-1,2,4-oxadiazol-3-yl)methyl)phenyl)methanone; N-(3-fluorobenzyl)-4-((5-(trifluoromethyl)-1,2,4-oxadiazol-3-yl)methyl)benzamide; N-(1-(p-tolyl)ethyl)-4-((5-(trifluoromethyl)-1,2,4-oxadiazol-3-yl)methyl)benzamide; N-(pyridin-3-ylmethyl)-4-((5-(trifluoromethyl)-1,2,4-oxadiazol-3-yl)methyl)benzamide; N-(5-chloropyridin-3-yl)-4-((5-(trifluoromethyl)-1,2,4-oxadiazol-3-yl)methyl)benzamide; N-(2-chloro-5-methoxyphenyl)-4-((5-(trifluoromethyl)-1,2,4-oxadiazol-3-yl)methyl)benzamide; N-(2-methoxyphenyl)-4-((5-(trifluoromethyl)-1,2,4-oxadiazol-3-yl)methyl)benzamide; N-(4-methoxyphenyl)-4-((5-(trifluoromethyl)-1,2,4-oxadiazol-3-yl)methyl)benzamide; N-(2-morpholinoethyl)-4-((5-(trifluoromethyl)-1,2,4-oxadiazol-3-yl)methyl)benzamide; N-(4-chlorophenyl)-4-((5-(trifluoromethyl)-1,2,4-oxadiazol-3-yl)methyl)benzamide; N-(3-fluorobenzyl)-N-methyl-4-((5-(trifluoromethyl)-1,2,4-oxadiazol-3-yl)methyl)benzamide; N-(isoxazol-3-yl)-4-((5-(trifluoromethyl)-1,2,4-oxadiazol-3-yl)methyl)benzamide; 4-methoxy-N-(4-(5-(trifluoromethyl)-1,2,4-oxadiazole-3-carbonyl)phenyl)benzamide; 4-chloro-N-(4-(5-(trifluoromethyl)-1,2,4-oxadiazole-3-carbonyl)phenyl)benzamide; N-(4-(5-(trifluoromethyl)-1,2,4-oxadiazole-3-carbonyl)phenyl)isonicotinamide; N-(4-(5-(trifluoromethyl)-1,2,4-oxadiazole-3-carbonyl)phenyl)nicotinamide; tert-butyl (4-(5-(trifluoromethyl)-1,2,4-oxadiazole-3-carbonyl)phenyl)carbamate; tert-butyl (4-(difluoro(5-(trifluoromethyl)-1,2,4-oxadiazol-3-yl)methyl)phenyl)carbamate; 2-(4-fluorophenyl)-N-(4-(5-(trifluoromethyl)-1,2,4-oxadiazole-3-carbonyl)phenyl)acetamide; N-(4-(difluoro(5-(trifluoromethyl)-1,2,4-oxadiazol-3-yl)methyl)phenyl)-4-(trifluoromethyl)benzamide; N-(4-(difluoro(5-(trifluoromethyl)-1,2,4-oxadiazol-3-yl)methyl)phenyl)-2-phenylacetamide; N-(4-(difluoro(5-(trifluoromethyl)-1,2,4-oxadiazol-3-yl)methyl)phenyl)-4-fluorobenzamide; N-(4-(difluoro(5-(trifluoromethyl)-1,2,4-oxadiazol-3-yl)methyl)phenyl)benzamide; N-(4-(difluoro(5-(trifluoromethyl)-1,2,4-oxadiazol-3-yl)methyl)phenyl)-2-(4-fluorophenyl)acetamide; 4-cyano-N-(4-(difluoro(5-(trifluoromethyl)-1,2,4-oxadiazol-3-yl)methyl)phenyl)benzamide; N-(4-(difluoro(5-(trifluoromethyl)-1,2,4-oxadiazol-3-yl)methyl)phenyl)-4-methylbenzamide; N-(4-(difluoro(5-(trifluoromethyl)-1,2,4-oxadiazol-3-yl)methyl)phenyl)picolinamide; N-methyl-4-((5-(trifluoromethyl)-1,2,4-oxadiazol-3-yl)methyl)benzamide; N,N-dimethyl-4-((5-(trifluoromethyl)-1,2,4-oxadiazol-3-yl)methyl)benzamide; N-(2-methoxyethyl)-4-((5-(trifluoromethyl)-1,2,4-oxadiazol-3-yl)methyl)benzamide; N-allyl-4-((5-(trifluoromethyl)-1,2,4-oxadiazol-3-yl)methyl)benzamide; azetidin-1-yl(4-((5-(trifluoromethyl)-1,2,4-oxadiazol-3-yl)methyl)phenyl)methanone; pyrrolidin-1-yl(4-((5-(trifluoromethyl)-1,2,4-oxadiazol-3-yl)methyl)phenyl)methanone; N-(2-methoxyethyl)-N-methyl-4-((5-(trifluoromethyl)-1,2,4-oxadiazol-3-yl)methyl)benzamide; N-(cyclopropylmethyl)-4-((5-(trifluoromethyl)-1,2,4-oxadiazol-3-yl)methyl)benzamide; N-ethyl-N-methyl-4-((5-(trifluoromethyl)-1,2,4-oxadiazol-3-yl)methyl)benzamide; N-allyl-N-methyl-4-((5-(trifluoromethyl)-1,2,4-oxadiazol-3-yl)methyl)benzamide; N-(prop-2-yn-1-yl)-4-((5-(trifluoromethyl)-1,2,4-oxadiazol-3-yl)methyl)benzamide; N-phenyl-4-((5-(trifluoromethyl)-1,2,4-oxadiazol-3-yl)methyl)benzamide; tert-butyl (4-((5-(trifluoromethyl)-1,2,4-oxadiazol-3-yl)methyl)phenyl)carbamate; N-(3,4-dichlorophenyl)-4-((5-(trifluoromethyl)-1,2,4-oxadiazol-3-yl)methyl)benzamide; N-(p-tolyl)-4-((5-(trifluoromethyl)-1,2,4-oxadiazol-3-yl)methyl)benzamide; N-(3-chlorophenyl)-4-((5-(trifluoromethyl)-1,2,4-oxadiazol-3-yl)methyl)benzamide; N-(4-(dimethylamino)phenyl)-4-((5-(trifluoromethyl)-1,2,4-oxadiazol-3-yl)methyl)benzamide; N-(4-(tert-butyl)phenyl)-4-((5-(trifluoromethyl)-1,2,4-oxadiazol-3-yl)methyl)benzamide; N-(m-tolyl)-4-((5-(trifluoromethyl)-1,2,4-oxadiazol-3-yl)methyl)benzamide; 4-((5-(trifluoromethyl)-1,2,4-oxadiazol-3-yl)methyl)-N-(3-(trifluoromethyl)phenyl)benzamide; N-(3-fluorophenyl)-4-((5-(trifluoromethyl)-1,2,4-oxadiazol-3-yl)methyl)benzamide; N-(2-fluorophenyl)-4-((5-(trifluoromethyl)-1,2,4-oxadiazol-3-yl)methyl)benzamide; N-(4-fluorophenyl)-4-((5-(trifluoromethyl)-1,2,4-oxadiazol-3-yl)methyl)benzamide; N-(2,4-dichlorophenyl)-4-((5-(trifluoromethyl)-1,2,4-oxadiazol-3-yl)methyl)benzamide; N-(m-tolyl)-4-((5-(trifluoromethyl)-1,2,4-oxadiazol-3-yl)methyl)benzothioamide; N-(4-(dimethylamino)phenyl)-4-((5-(trifluoromethyl)-1,2,4-oxadiazol-3-yl)methyl)benzothioamide; N-(3-fluorophenyl)-4-((5-(trifluoromethyl)-1,2,4-oxadiazol-3-yl)methyl)benzothioamide; N-(4-fluorophenyl)-4-((5-(trifluoromethyl)-1,2,4-oxadiazol-3-yl)methyl)benzothioamide; N-(4-((5-(trifluoromethyl)-1,2,4-oxadiazol-3-yl)methyl)phenyl)benzenesulfonamide; 4-fluoro-N-(4-((5-(trifluoromethyl)-1,2,4-oxadiazol-3-yl)methyl)phenyl)benzenesulfonamide; 4-methyl-N-(4-((5-(trifluoromethyl)-1,2,4-oxadiazol-3-yl)methyl)phenyl)benzenesulfonamide; N-(3-fluorobenzyl)-4-((5-(trifluoromethyl)-1,2,4-oxadiazol-3-yl)methyl)benzothioamide; 3-chloro-N-(4-((5-(trifluoromethyl)-1,2,4-oxadiazol-3-yl)methyl)phenyl)benzenesulfonamide; 1-isopropyl-3-(4-((5-(trifluoromethyl)-1,2,4-oxadiazol-3-yl)methyl)phenyl)urea; 1-(pyridin-3-yl)-3-(4-((5-(trifluoromethyl)-1,2,4-oxadiazol-3-yl)methyl)phenyl)urea; 1-(4-methoxyphenyl)-3-(4-((5-(trifluoromethyl)-1,2,4-oxadiazol-3-yl)methyl)phenyl)urea; 1-(p-tolyl)-3-(4-((5-(trifluoromethyl)-1,2,4-oxadiazol-3-yl)methyl)phenyl)urea; 1-(4-chlorophenyl)-3-(4-((5-(trifluoromethyl)-1,2,4-oxadiazol-3-yl)methyl)phenyl)urea; 1-(4-fluorophenyl)-3-(4-((5-(trifluoromethyl)-1,2,4-oxadiazol-3-yl)methyl)phenyl)urea; 2-fluoro-N-(4-((5-(trifluoromethyl)-1,2,4-oxadiazol-3-yl)methyl)phenyl)benzenesulfonamide; 1-phenyl-3-(4-((5-(trifluoromethyl)-1,2,4-oxadiazol-3-yl)methyl)phenyl)urea; 1-ethyl-3-(4-((5-(trifluoromethyl)-1,2,4-oxadiazol-3-yl)methyl)phenyl)urea; N-phenyl-4-(2-(5-(trifluoromethyl)-1,2,4-oxadiazol-3-yl)propan-2-yl)benzamide; N-(p-tolyl)-4-(2-(5-(trifluoromethyl)-1,2,4-oxadiazol-3-yl)propan-2-yl)benzamide; N-(4-chlorophenyl)-4-(2-(5-(trifluoromethyl)-1,2,4-oxadiazol-3-yl)propan-2-yl)benzamide; N-(pyridin-4-yl)-4-(2-(5-(trifluoromethyl)-1,2,4-oxadiazol-3-yl)propan-2-yl)benzamide; 3-(trifluoromethyl)-N-(4-((5-(trifluoromethyl)-1,2,4-oxadiazol-3-yl)methyl)phenyl)benzenesulfonamide; N-(2-methoxyphenyl)-4-(2-(5-(trifluoromethyl)-1,2,4-oxadiazol-3-yl)propan-2-yl)benzamide; N-(pyridin-3-yl)-4-(2-(5-(trifluoromethyl)-1,2,4-oxadiazol-3-yl)propan-2-yl)benzamide; 1-(cyclopropylmethyl)-3-(4-((5-(trifluoromethyl)-1,2,4-oxadiazol-3-yl)methyl)phenyl)urea; 1-(tert-butyl)-3-(4-((5-(trifluoromethyl)-1,2,4-oxadiazol-3-yl)methyl)phenyl)urea; phenyl (4-((5-(trifluoromethyl)-1,2,4-oxadiazol-3-yl)methyl)phenyl)carbamate; methyl (4-((5-(trifluoromethyl)-1,2,4-oxadiazol-3-yl)methyl)phenyl)carbamate; N-(4-((5-(trifluoromethyl)-1,2,4-oxadiazol-3-yl)methyl)benzyl)cyclopropanecarboxamide; 4-methyl-N-(4-((5-(trifluoromethyl)-1,2,4-oxadiazol-3-yl)methyl)benzyl)benzamide; 2-fluoro-N-(4-((5-(trifluoromethyl)-1,2,4-oxadiazol-3-yl)methyl)benzyl)benzamide; 3-fluoro-N-(4-((5-(trifluoromethyl)-1,2,4-oxadiazol-3-yl)methyl)benzyl)benzamide; 3-chloro-N-(4-((5-(trifluoromethyl)-1,2,4-oxadiazol-3-yl)methyl)benzyl)benzamide; N-(4-((5-(trifluoromethyl)-1,2,4-oxadiazol-3-yl)methyl)benzyl)propionamide; N-phenyl-4-(1-(5-(trifluoromethyl)-1,2,4-oxadiazol-3-yl)cyclopropyl)benzamide; N-(p-tolyl)-4-(1-(5-(trifluoromethyl)-1,2,4-oxadiazol-3-yl)cyclopropyl)benzamide; N-(4-chlorophenyl)-4-(1-(5-(trifluoromethyl)-1,2,4-oxadiazol-3-yl)cyclopropyl)benzamide; N-(2-methoxyphenyl)-4-(1-(5-(trifluoromethyl)-1,2,4-oxadiazol-3-yl)cyclopropyl)benzamide; 3-(4-((phenylthio)methyl)benzyl)-5-(trifluoromethyl)-1,2,4-oxadiazole; 3-(4-((phenylsulfinyl)methyl)benzyl)-5-(trifluoromethyl)-1,2,4-oxadiazole; and 3-(4-((phenylsulfonyl)methyl)benzyl)-5-(trifluoromethyl)-1,2,4-oxadiazole.

The present invention also relates to a process for preparing a compound of Formula I. A person skilled in the art can easily practice all or any of the following steps to prepare the compound of Formula I:
a. reacting a nitrile derivative of Formula (i) with hydroxylamine salt in the presence of a suitable base to obtain hydroxyl imidamide derivative of Formula (ii),

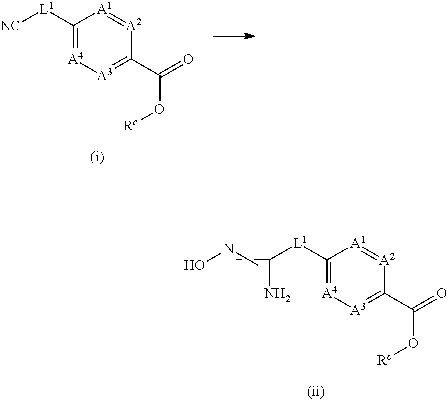

wherein, R^{c} is C₁-C₄-alkyl; L¹ is CR⁴R⁵; and R⁴, R⁵, A¹, A², A³, and A⁴ are as define hereinabove;
b. reacting the hydroxyl imidamide derivative of Formula (ii) with an anhydride of Formula (V-a) or an acid halide of Formula (V-b) to obtain a compound of Formula (iii),

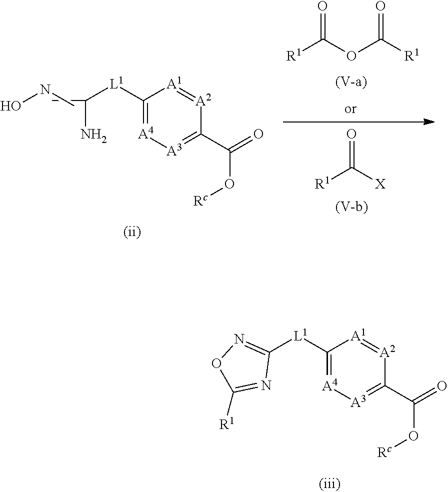

wherein, R^{c} is C₁-C₄-alkyl; L¹ is CR⁴R⁵; X is halide; and R¹, R⁴, R⁵, A¹, A², A³, and A⁴ are as define hereinabove;
c. reacting the compound of Formula (iii) with an amine in the presence of trialkyl aluminium to obtain the compound of Formula I,

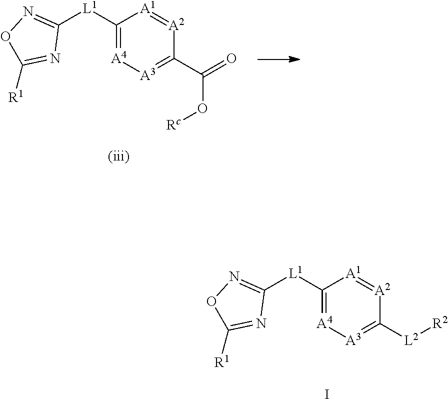

wherein, R^{c} is C₁-C₄-alkyl; L¹ is CR⁴R⁵; L² is C(═O)NR⁶; and R¹, R², R⁴, R⁵, R⁶, A¹, A², A³, and A⁴ are as define hereinabove;
d. reacting a nitrile derivative of Formula (iv) with hydroxylamine salt in the presence of a suitable base to obtain hydroxyl imidamide derivative of Formula (v),

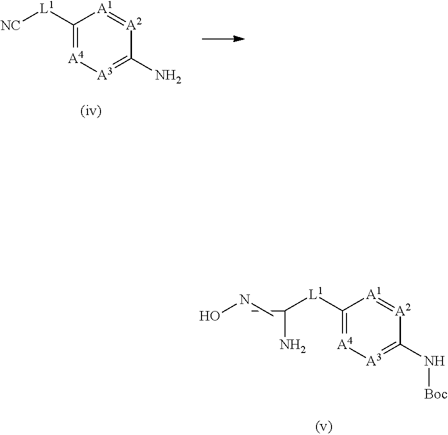

wherein, L¹ is CR⁴R⁵, C(═W) or CF₂; and R⁴, R⁵, W, A¹, A², A³, and A⁴ are as define hereinabove;
e. reacting the hydroxyl imidamide derivative of Formula (v) with an anhydride of Formula (V-a) or an acid halide of Formula (V-b) to obtain a compound of Formula (vi),

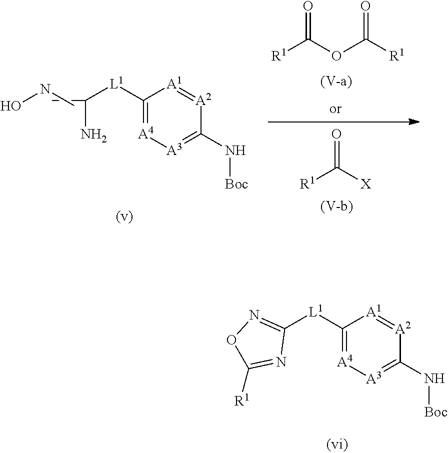

wherein, L¹ is CR⁴R⁵, C(═W) and CF₂; X is halide; and R¹, R⁴, R⁵, W, A¹, A², A³, and A⁴ are as define hereinabove;
f. converting the compound of Formula (vi) into the compound of Formula (vii) using a suitable reagent,

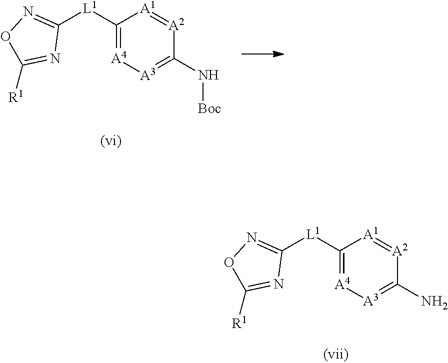

wherein, L¹ is CR⁴R⁵, C(═W) and CF₂; and R¹, R⁴, R⁵, W, A¹, A², A³, and A⁴ are as define hereinabove;
g. reacting the compound of Formula (vii) with a suitable reactant to obtain the compound of Formula I,

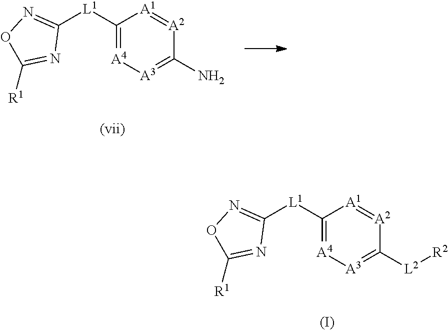

wherein, the suitable reactant is acid or acid halide when L² is NR⁶C(═O); L¹ is CR⁴R⁵, C(═W) and CF₂; and R¹, R², R⁴, R⁵, R⁶, W, A¹, A², A³, and A⁴ are as define hereinabove, and the reaction is carried out using a suitable base optionally in the presence of a suitable coupling reagent;
the suitable reactant is sulphonyl chloride when L² is NR⁶; R² is selected from the group consisting of C₁-C₆-haloalkylsulfonyl, arylsulfonyl, heteroarylsulfonyl, C₃-C₈-cycloalkylsulfonyl C₁-C₆-alkylsulfinyl, and C₁-C₆-alkylsulfonyl; L¹ is CR⁴R⁵, C(═W) and CF₂; and R¹, R⁴, R⁵, R⁶, W, A¹, A², A³, and A⁴ are as define hereinabove, and the reaction is carried out using a suitable base;
the suitable reactant is hydroxy compound when L² is NR⁶C(═O); R² is C₁-C₆-alkoxy, aryloxy, heteroaryloxy, C₃-C₈-heterocylyloxy, C₃-C₈-cycloalkyloxy, and C₁-C₆-haloalkoxy; L¹ is CR⁴R⁵, C(═W) and CF₂; and R¹, R⁴, R⁵, R⁶, W, A¹, A², A³, and A⁴ are as define hereinabove, and the reaction is carried out using a suitable reagent;
the suitable reactant is amine compound when L² is NR⁶C(═O); R² is C₁-C₆-alkylamino, arylamino, heteroarylamino, C₄-C₈-heterocyclylamino, C₁-C₆-dialkylamino, C₃-C₈-cycloalkylamino, and C₁-C₆-alkyl-C₃-C₈-cycloalkylamino; L¹ is CR⁴R⁵, C(═W) and CF₂; and R¹, R⁴, R⁵, R⁶, W, A¹, A², A³, and A⁴ are as define hereinabove, and the reaction is carried out using a suitable reagent;
h. fluorinating a compound of Formula (d) to obtain the compound of Formula (vi) using a suitable fluorinating agent,

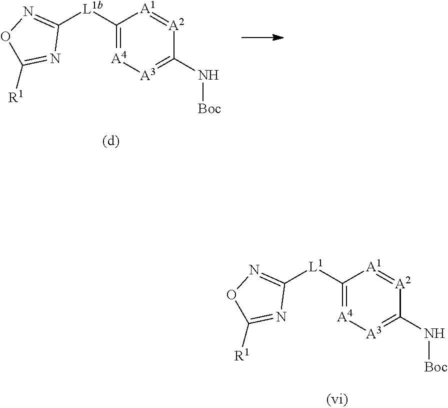

wherein, L¹ is CF₂; L^{1b} is C(═O); and R¹, A¹, A², A³, and A⁴ are as define hereinabove;
i. reacting a nitrile derivative of Formula (g) with hydroxylamine salt in the presence of a suitable base to obtain hydroxyl imidamide derivative of Formula (h),

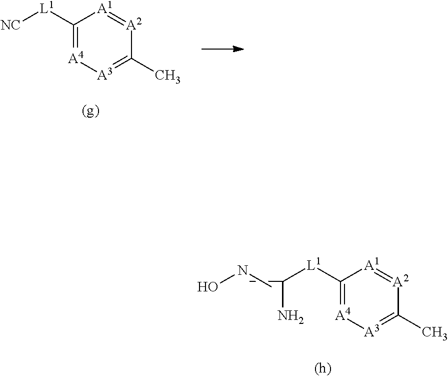

wherein, L¹ is CR⁴R⁵; and R⁴, R⁵, A¹, A², A³, and A⁴ are as define hereinabove;
j. reacting the hydroxyl imidamide derivative of Formula (h) with an anhydride of Formula (V-a) or an acid halide of Formula (V-b) to obtain a compound of Formula (i),

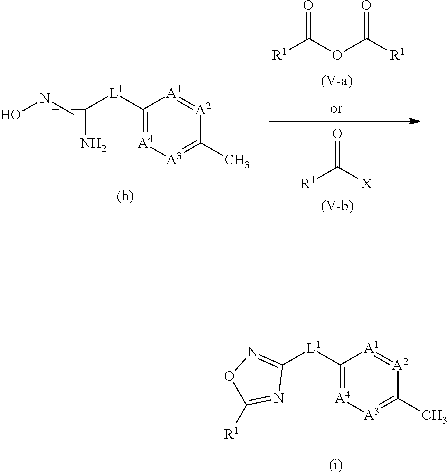

wherein, L¹ is CR⁴R⁵; X is halide; and R¹, R⁴, R⁵, A¹, A², A³, and A⁴ are as define hereinabove;
k. brominating the compound of Formula (i) using a suitable brominating reagent in the presence of a suitable radical initiator to obtain a compound of Formula (j),

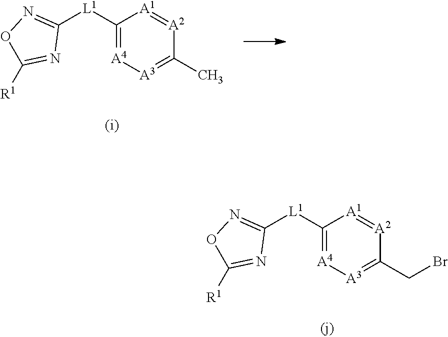

wherein, L¹ is CR⁴R⁵; and R¹, R⁴, R⁵, A¹, A², A³, and A⁴ are as define hereinabove;
l. converting the compound of Formula (j) into a compound of Formula (k) using a suitable metal azide,

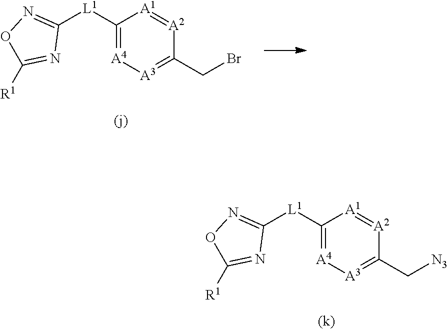

wherein, L¹ is CR⁴R⁵; and R¹, R⁴, R⁵, A¹, A², A³, and A⁴ are as define hereinabove;
m. reducing the compound of Formula (k) into a compound of Formula (l) using a suitable phosphine reagent,

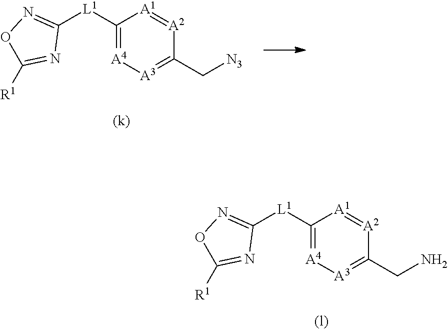

wherein, L¹ is CR⁴R⁵; and R¹, R⁴, R⁵, A¹, A², A³, and A⁴ are as define hereinabove;
n. reacting the compound of Formula (I) with a suitable reactant to obtain the compound of Formula I,

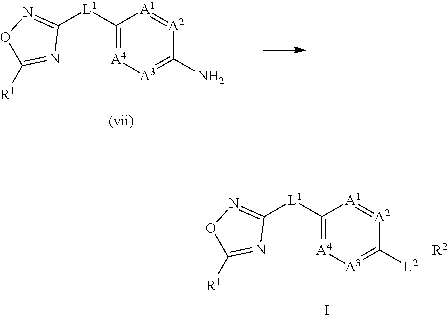

wherein, the suitable reactant is acid or acid halide when L² is CR⁴R⁵; R² is selected from the group consisting of C₁-C₆-alkylcarbonylamino, C₃-C₆-cycloalkylcarbonylamino, arylcarbonylamino, heteroarylcarbonylamino, heterocyclylcarbonylamino, and 1 C₆-haloalkylcarbonylamino, L¹ is CR⁴R⁵; and R¹, R⁴, R⁵, A¹, A², A³, and A⁴ are as define hereinabove, and the reaction is carried out using a suitable base optionally in the presence of a suitable coupling reagent;
the suitable reactant is sulphonyl chloride when L² is CR⁴R⁵; R² is sulfonamide; L¹ is CR⁴R⁵; and R¹, R⁴, R⁵, A¹, A², A³, and A⁴ are as define hereinabove, and the reaction is carried out using a suitable base;
the suitable reactant is isocyanate compound when L² is CR⁴R⁵; and R² is selected from the group consisting of C₁-C₆-alkylaminocarbonylamino, C₁-C₆-dialkylaminocarbonylamino, arylaminocarbonylamino, heteroarylaminocarbonylamino, and C₃-C₆-cycloalkylaminocarbonylamino; L¹ is CR⁴R⁵; and R¹, R⁴, R⁵, A¹, A², A³, and A⁴ are as define hereinabove;
the suitable reactant is chloroformate compound when L² is CR⁴R⁵; and R² is selected from the group consisting of C₁-C₆-alkyloxycarbonylamino, aryloxycarbonylamino, heterocycloxycarbonylamino, heteroaryloxycarbonylamino, and C₃-C₆-cycloalkyloxycarbonylamino; L¹ is CR⁴R⁵; and R¹, R⁴, R⁵, A¹, A², A³, and A⁴ are as define hereinabove, and the reaction is carried out using a suitable reagent;
o. brominating the compound of Formula (g) using a suitable brominating reagent in the presence of a suitable radical initiator to obtain a compound of Formula (m),

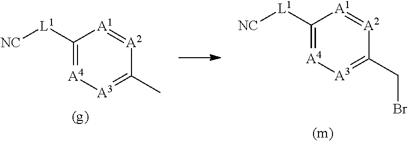

wherein, L¹ is CR⁴R⁵; and R⁴, R⁵, A¹, A², A³, and A⁴ are as define hereinabove;
p. reacting the compound of Formula (m) with a mercapto compound in the presence of a suitable base,

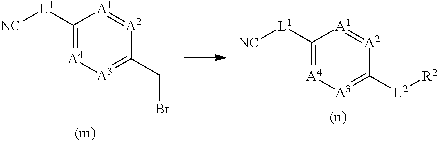

wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio; and R⁴, R⁵, A¹, A², A³, and A⁴ are as define hereinabove;
q. reacting a nitrile derivative of Formula (n) with hydroxylamine salt in the presence of a suitable base to obtain hydroxyl imidamide derivative of Formula (o),

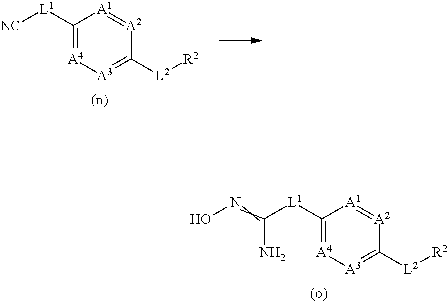

wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio; and R⁴, R⁵, A¹, A², A³, and A⁴ are as define hereinabove;
r. reacting the hydroxyl imidamide derivative of Formula (o) with an anhydride of Formula (V-a) or an acid halide of Formula (V-b) to obtain the compound of Formula I,

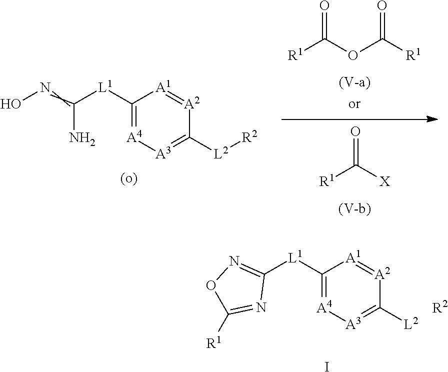

wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio; X is halide; and R¹, R⁴, R⁵, A¹, A², A³, and A⁴ are as define hereinabove;
s. oxidizing a compound of Formula (p) to obtain the compound of Formula I using a suitable oxidizing reagent,

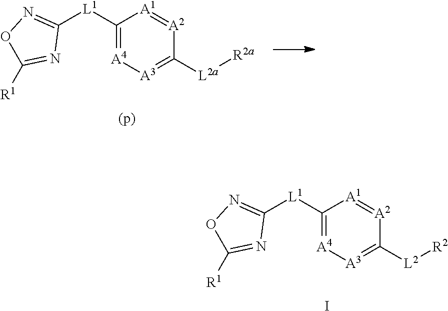

wherein, L¹ is CR⁴R⁵; L² is CH₂; L^{2a} is CH₂; R^{2a} is selected from the group consisting of C₁-C₆-alkylthio, arylthio, heteroarylthio, C₄-C₅-heterocyclylthio, and C₁-C₆-haloalkylthio; R² is selected from the group consisting of C₁-C₆-haloalkylsulfinyl, arylsulfinyl, heteroarylsulfinyl, C₃-C₈-cycloalkylsulfinyl, C₁-C₆-alkylsulfinyl, C₁-C₆-haloalkylsulfonyl, arylsulfonyl, heteroarylsulfonyl, C₃-C₈-cycloalkylsulfonyl, and C₁-C₆-alkylsulfonyl; and R⁴, R⁵, A¹, A², A³, and A⁴ are as define hereinabove;
t. hydrolyzing an ester of Formula (q) into an acid of Formula (r) using suitable hydrolyzing agent,

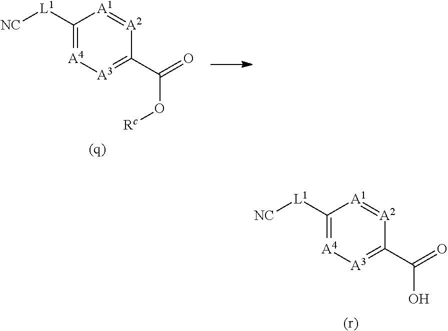

wherein, R^{c} is C₁₋C₄-alkyl; L¹ is CR⁴R⁵; and R⁴, R⁵, A¹, A², A³, and A⁴ are as define hereinabove;
u. the acid of Formula (r) is reacted with hydroxylamine salt in the presence of a suitable base to obtain the compound of Formula (s),

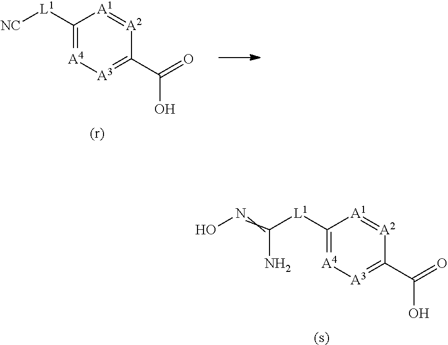

wherein, L¹ is CR⁴R⁵; and R⁴, R⁵, A¹, A², A³, and A⁴ are as define hereinabove;
v. reacting the compound of Formula (s) with an anhydride of Formula (V-a) or an acid halide of Formula (V-b) to obtain the compound of Formula (t),

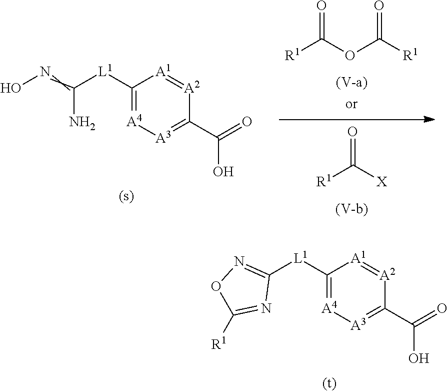

wherein, L¹ is CR⁴R⁵; X is halide; R¹, R⁴, R⁵, A¹, A², A³, and A⁴ are as define hereinabove;
w. reacting the compound of Formula (t) with an amine NHR⁶R² in the presence of a suitable coupling reagent and a suitable base to obtain the compound of Formula I,

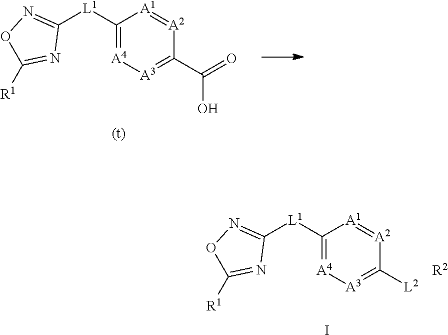

wherein, L¹ is CR⁴R⁵; L² is C(═O)NR⁶; R¹, R², R⁴, R⁵, R⁶, A¹, A², A³, and A⁴ are as define hereinabove; and
x. converting a compound of Formula (u) into the compound of Formula I using a Lawesson's reagent,

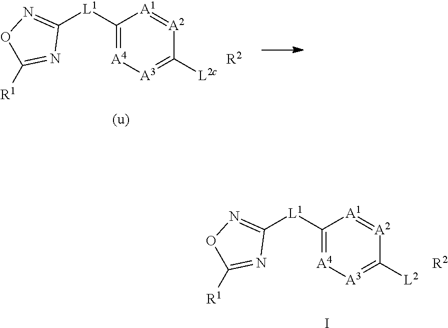

wherein, L¹ is CR⁴R⁵; L² is C(═S); R² is selected from the group consisting of C₁-C₆-alkylamino, arylamino, heteroarylamino, C₄-C₈-heterocyclylamino, C₁-C₆-dialkylamino, C₃-C₈-cycloalkylamino, and C₁-C₆-alkyl-C₃-C₈-cycloalkylamino; L^{2c} is C(═O); and R¹, R⁴, R⁵, A¹, A², A³, and A⁴ are as define hereinabove.

The present invention also relates intermediate compound of Formula (ii),

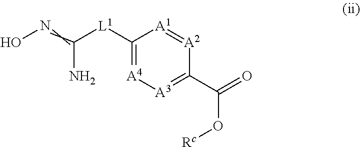

wherein, L¹ is —C(R⁴R⁵)— or —C(═W)—; R⁴ & R⁵ are as define hereinabove excluding hydrogen; R^{c} is C1-C4-alkyl; and A¹, A², A³, and A⁴ are as define hereinabove.

The present invention also relates other intermediate compounds of Formulae (vi), (vii), (k), and (t) which can be used for preparing the compound of Formula I;

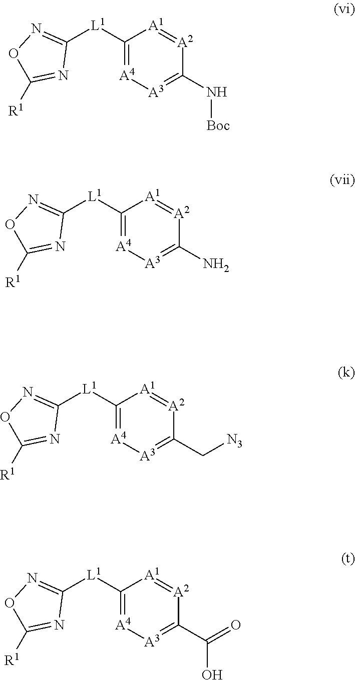

wherein, L¹ is —C(R⁴R⁵)— or —C(═W)—; R¹ is CF₃, CF₂Cl or CHF₂ and R⁴, R⁵ A¹, A², A³, and A⁴ are as define hereinabove.

The compound of the present invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compound of the present invention may be present as a mixture of stereoisomers, individual stereoisomers or as an optically active form.

An anion part of the salt in case the compound of Formula I is a cationic or capable of forming a cation can be inorganic or organic. Alternatively, a cation part of the salt in case the compound of Formula I is an anionic or capable of forming anion can be inorganic or organic. Examples of inorganic anion part of the salt include but are not limited to chloride, bromide, iodide, fluoride, sulphate, phosphate, nitrate, nitrite, hydrogen carbonates, hydrogen sulphate. Examples of organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkylphosphonates, arylphosphonates, aryldiphosphonates, p-toluenesulphonate, and salicylate. Examples of inorganic cation part of the salt include but are not limited to alkali and alkaline earth metals. Examples of organic cation part of the salt include but are not limited to pyridine, methyl amine, imidazole, benzimidazole, hitidine, phosphazene, tetramethyl ammonium, tetrabutylammonium, choline and trimethylamine.

Metal ions in metal complexes of the compound of Formula I are especially the ions of the elements of the second main group, especially calcium and magnesium, of the third and fourth main group, especially aluminium, tin and lead, and also of the first to eighth transition groups, especially chromium, manganese, iron, cobalt, nickel, copper, zinc and others. Particular preference is given to the metal ions of the elements of the fourth period and the first to eighth transition groups. Here, the metals can be present in the various valencies that they can assume.

The compound selected from Formula I, (including all stereoisomers, N-oxides, and salts thereof), typically may exist in more than one form. Formula I thus includes all crystalline and non-crystalline forms of the compound that Formula I represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula I can exhibit beneficial effects (e.g., suitability for preparation of useful formulations, improved biological performance) relative to another polymorph or a mixture of polymorphs of the same compound represented by Formula I. Preparation and isolation of a particular polymorph of a compound represented by Formula I can be achieved by methods known to those skilled in the art including, for example, crystallization using selected solvents and temperatures.

In another embodiment the present invention relates to a composition comprising the compound of Formula I, agriculturally acceptable salts, metal complexes, constitutional isomers, stereo-isomers, diastereoisomers, enantiomers, chiral isomers, atropisomers, conformers, rotamers, tautomers, optical isomers, polymorphs, geometric isomers, or N-oxides thereof optionally with one or more additional active ingredient with the auxiliary such as inert carrier or any other essential ingredient such as surfactants, additives, solid diluents and liquid diluents.

The compound of Formula I and the composition according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compound of Formula I and the composition according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Particularly, the compound of Formula I and the composition according to the invention are important in the control of phytopathogenic fungi on soybeans and on the plant propagation material, such as seeds, and the crop material of soybeans. Accordingly, the present invention also includes a composition comprising at least one compound of Formula I and seed. The amount of the compound of Formula I in the composition ranges from 0.1 gai (gram per active ingredient) to 10 kgai (kilogram per active ingredient) per 100 kg of seeds.

Preferably, the compound of Formula I and composition thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative or reproductive parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts, twigs, flowers, and other parts of plants, including seedlings and young plants, which are to be transplanted after germination or after emergence from soil.

These young plants may also be protected before transplantation by a total or partial treatment by immersion or pouring.

Preferably, treatment of plant propagation materials with the compound of Formula I, the combination and or the composition thereof, respectively, is used for controlling a multitude of fungi on cereals, such as wheat, rye, barley and oats; rice, corn, cotton, fruits, coffee, sugarcane and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant.

Such genetic modifications also include but are not limited to targeted post-translational modification of protein(s), oligo- or polypeptides e. g. by glycosylation or polymer additions such as prenylated, acetylated or farnesylated moieties or PEG moieties. Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1 185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants capable to synthesize one or more insecticidal proteins, especially those known from the bacterial genus (Bacillus), by the use of recombinant DNA techniques are within the scope of the present invention. The Bacillus are particularly from Bacillus thuringiensis, such as δ-endotoxins, e. g. Cry1A(b), Cry1A(c), Cry1F, Cry1F(a2), Cry11A(b), Cry111A, Cry111B(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO02/015701). Further examples of such toxins or genetically modified plants capable of synthesizing such toxins are disclosed, e. g., in EP374753, WO93/007278, WO95/34656, EP427 529, EP451 878, WO03/18810 und WO03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the Cry1Ac toxin), Bollgard® I (cotton cultivars producing the Cry1 Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt1 1 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1 F toxin and PAT enzyme).

Furthermore, plants capable to synthesize one or more proteins to increase the resistance or tolerance of those plants to bacterial, viral or fungal pathogens by the use of recombinant DNA techniques are also within the scope of the present invention. Examples of such proteins are the so-called “pathogenesis-related proteins” (PR proteins, see, e. g. EP392225), plant disease resistance genes (e. g. potato cultivars, which express resistance genes acting against Phytophthora infestans derived from the Mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants capable to synthesize one or more proteins, by the use of recombinant DNA techniques, to increase the productivity (e. g. bio mass production, grain yield, starch content, oil content or protein content), tolerance to drought, salinity or other growth-limiting environmental factors or tolerance to pests and fungal, bacterial or viral pathogens of those plants are within the scope of the present invention.

Furthermore, plants that contain a modified amount of substances of content or new substances of content, by the use of recombinant DNA techniques, to improve human or animal nutrition, e. g. oil crops that produce health-promoting long-chain omega-3 fatty acids or unsaturated omega-9 fatty acids (e. g. Nexera® rape, DOW Agro Sciences, Canada) are also within the scope of the present invention.

Furthermore, plants that contain a modified amount of substances of content or new substances of content, by the use of recombinant DNA techniques, to improve raw material production, e. g. potatoes that produce increased amounts of amylopectin (e. g. Amflora® potato, BASF SE, Germany) are also within the scope of the present invention.

The present invention also relates to a method for controlling or preventing infestation of plants by phytopathogenic micro-organisms in agricultural crops and or horticultural crops wherein an effective amount of at least one compound of Formula I or the combination of the present invention or the composition of the present invention, is applied to the seeds of plants. The compound, the combination and the composition of the present invention can be used for controlling or preventing plant diseases. The compound of Formula I, the combination and or the composition thereof, respectively, are particularly suitable for controlling the following plant diseases:

Albugo spp. (white rust) on ornamentals, vegetables (e. g. A. Candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (C. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (£. pyri), soft fruits (£. veneta: anthracnose) and vines (£. ampelina: anthracnose); Entyloma oryzae (leaf smut) on rice; Epicoccum spp. (black mold) on wheat; Erysiphe spp. (powdery mildew) on sugar beets (£. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), soybeans, potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or.rotbrenner′, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compound of Formula I, the combination or the composition thereof may be used to treat several fungal pathogens. Non-limiting examples of pathogens of fungal diseases which can be treated in accordance with the invention include:

Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, ltersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries. Blastocladiomycetes, such as Physoderma maydis. Mucoromycetes, such as Choanephora cucurbitarum.; Mucor spp.; and Rhizopus arrhizus,

In another embodiment diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

In particular, Cronartium ribicola (White pine blister rust); Gymnosporangium juniperi-virginianae (Cedar-apple rust); Hemileia vastatrix (Coffee rust); Phakopsora meibomiae and P. pachyrhizi (Soybean rust); Puccinia coronata (Crown Rust of Oats and Ryegrass); Puccinia graminis (Stem rust of wheat and Kentucky bluegrass, or black rust of cereals); Puccinia hemerocallidis (Daylily rust); Puccinia persistens subsp. triticina (wheat rust or ‘brown or red rust’); Puccinia sorghi (rust in corn); Puccinia striiformis (‘Yellow rust’ in cereals); Uromyces appendiculatus (rust of beans); Uromyces phaseoli (Bean rust); Puccinia melanocephala (‘Brown rust’ in sugarcane); Puccinia kuehnii (‘Orange rust’ in sugarcane).

Plants which can be treated in accordance with the invention include the following: cotton, flax, grapevine, fruits, vegetables, such as Rosaceae sp (for example pome fruits such as apples, pears, apricots, cherries, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Vitaceae sp. (for example grapes); Solanaceae sp. (for example tomatoes, peppers), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Poaceae/Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); Malvaceae (for example cotton); useful plants and ornamental plants for gardens and wooded areas; and genetically modified varieties of each of these plants.

More preference is given to controlling the following diseases of soya beans: Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), Choanephora leaf blight (Choanephora infundibulifera trispora (Syn.)), Dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), Phyllosticta leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectiia crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), Mycoleptodiscus root rot (Mycoleptodiscus terrestris), Neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidennatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The present invention also relates to the use of the compound of Formula I, the combination or the composition thereof for controlling or preventing the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye and Phakopsoraceae spp. on various plants, in particular Phakopsora pachyrhizi and P. meibomiae (soybean rust) on soybeans, Hemileia vastatrix (Coffee rust), Uromyces appendiculatus, Uromyces fabae and Uromyces phaseoli (rust of beans).

The present invention further relates to the use of the compound of Formula I, the combination or the composition thereof for controlling or preventing against phytopathogenic fungi such as Phakopsora pachyrhizi, Phakopsora meibomiae, of agricultural crops and or horticultural crops.

The compound of Formula I, the combination and the composition thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria.

As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Pora spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

In one embodiment the compound of Formula I, the combination and the composition thereof, respectively, are particularly suitable for controlling the following plant diseases: Phakopsora pachyrhizi and P. meibomiae (soybean rust) on soybeans.

The present invention further relates to a method for controlling or preventing phytopathogenic fungi. The method comprises treating the fungi or the materials, plants, plant parts, locus thereof, soil or seeds to be protected against fungal attack, with an effective amount of at least one compound of Formula I or the combination or the composition comprising at least one compound of Formula I.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combination according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compound of Formula I, the combination and the composition thereof, respectively, may be used for improving the health of a plant. The invention also relates to a method for improving plant health by treating a plant, its propagation material and/or the locus where the plant is growing or is to grow with an effective amount of compound I and the composition thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compound of Formula I can be present in different crystal modifications or polymorphs whose biological activity may differ. They are likewise subject matter of the present invention.

The compound of Formula I are employed as such or in the form of composition for treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with a compound of Formula I, the combination and the composition thereof protectively either at or before planting or transplanting.

The invention also relates to agrochemical composition comprising an auxiliary and at least one compound of Formula I.

An agrochemical composition comprises a fungicidally effective amount of a compound of Formula I. The term “effective amount” denotes an amount of the composition or of the compound of Formula I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound of Formula I used.

The compound of Formula I, their oxides, metal complexes, isomers, polymorphs or the agriculturally acceptable salts thereof can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel Formulations for the treatment of plant propagation materials such as seeds (e. g. GF). These and further compositions types are defined in the “Catalogue of pesticide Formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product Formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are mineral earths, e. g. silicates, silica gels, talc, kaolins, limestone, lime, chalk, clays, dolomite, diatomaceous earth, bentonite, calcium sulphate, magnesium sulphate, magnesium oxide; polysaccharides, e. g. cellulose, starch; fertilizers, e. g. ammonium sulphate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulphates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulphates are sulphates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound of Formula I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound of Formula I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible Concentrates (DC)

5-25 wt % of a compound of Formula I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound of Formula I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound of Formula I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound of Formula I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension.

Dilution with water gives a stable suspension of the active substance. For FS type composition up to 40 wt % binder (e. g. polyvinyl alcohol) is added.

vi) Water-Dispersible Granules and Water-Soluble Granules (WG, SG)

50-80 wt % of a compound of Formula I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance. vii) Water-dispersible powders and water-soluble powders (WP, SP, WS) 50-80 wt % of a compound of Formula I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound of Formula I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound of Formula I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound of Formula I, 0-40 wt % water insoluble organic solvent

(e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound of Formula I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable Powders (DP, DS)

1-10 wt % of a compound of Formula I are ground finely and mixed intimately with solid carrier (e. g. finely divided kaolin) ad 100 wt %.

xii) Granules (GR, FG)

0.5-30 wt % of a compound of Formula I are ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound of Formula I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active ingredient (ai). The active ingredients (ai) are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations.

Application can be carried out before or during sowing. Methods for applying the compound of Formula I, the combination and the composition thereof, respectively, onto plant propagation material, especially seeds, include dressing, coating, pelleting, dusting, and soaking as well as in-furrow application methods. Preferably, the compound of Formula I, the combination and the composition thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.05 to 1 kg per ha, more preferably from 0.1 to 1.0 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kg of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be mixed with the composition according to the invention in a weight ratio of 1:100 to 100:1, preferably 1:20 to 20:1.

A pesticide is generally a chemical or biological agent (such as pesticidally active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

The compound of Formula I, the combination and the composition thereof comprising them in the use as fungicides with other fungicides may result in an expansion of the fungicidal spectrum of activity being obtained or in a prevention of fungicide resistance development. Furthermore, in many cases, extraordinary effects are obtained.

The present invention also relates to the combination comprising at least one compound of Formula I and at least one further pesticidally active substance selected from the group of fungicides, insecticides, nematicides, acaricides, biopesticides, herbicides, safeners, plant growth regulators, antibiotics, fertiliers and nutrients. The pesticidally active substances reported in WO2015185485 pages 36-43 and WO2017093019 pages 42-56 can be used in conjunction with which the compound of Formula I.

The active substances referred to as component 2, their preparation and their activity e. g. against harmful fungi is known (cf.: http://www.alanwood.net/pesticides/); these substances are commercially available. The compounds described by IU PAC nomenclature, their preparation and their pesticidal activity are also known (cf. Can. J. Plant Sci. 48(6), 587-94, 1968; EP141317; EP152031; EP226917; EP243970; EP256503; EP428941; EP532022; EP1028125; EP1035122; EP1201648; EP1122244, JP2002316902; DE19650197; DE10021412; DE102005009458; U.S. Pat. Nos. 3,296,272; 3,325,503; WO9846608; WO9914187; WO9924413; WO9927783; WO0029404; WO0046148; WO0065913; WO0154501; WO 0156358; WO0222583; WO0240431; WO0310149; WO0311853; WO0314103; WO0316286; WO0353145; WO0361388; WO0366609; WO0374491; WO0449804; WO0483193; WO05120234; WO05123689; WO05123690; WO0563721; WO0587772; WO0587773; WO0615866; WO0687325; WO0687343; WO0782098; WO0790624; WO11028657; WO2012168188; WO2007006670; WO201177514; WO13047749; WO10069882; WO13047441; WO0316303; WO0990181; WO13007767; WO1310862; WO13127704; WO13024009; WO13024010; WO13047441; WO13162072; WO13092224 and WO11135833.

The present invention furthermore relates to agrochemical mixtures comprising at least one compound of Formula I (component 1) and at least one further active substance useful for plant protection.

By applying the compound of Formula I together with at least one pesticidally active compound an additional effect can be obtained.

This can be obtained by applying the compound of Formula I and at least one further pesticidally active substance simultaneously, either jointly (e. g. as tank-mix) or separately, or in succession, wherein the time interval between the individual applications is selected to ensure that the active substance applied first still occurs at the site of action in a sufficient amount at the time of application of the further pesticidally active substance(s). The order of application is not essential for working of the present invention.

When applying the compound of Formula I and a pesticidally active substance sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In the binary mixtures and the composition according to the invention the weight ratio of the component 1) and the component 2) generally depends from the properties of the active components used, usually it is in the range of 1:1000 to 1000:1, often in the range of 1:100 to 100:1, regularly in the range of 1:50 to 50:1, preferably in the range of 1:20 to 20:1, more preferably in the range of 1:10 to 10:1, even more preferably in the range of 1:4 to 4:1 and in particular in the range of 1:2 to 2:1.

According to a further embodiment of the binary mixtures and the composition thereof, the weight ratio of the component 1) and the component 2) usually is in the range of 1000:1 to 1:1000, often in the range of 100:1 to 1:100, regularly in the range of 50:1 to 1:50, preferably in the range of 20:1 to 1:20, more preferably in the range of 10:1 to 1:10, even more preferably in the range of 4:1 to 1:4 and in particular in the range of 2:1 to 1:2.

In the ternary mixtures, i.e. the composition according to the invention comprising the component 1) and component 2) and a compound III (component 3), the weight ratio of component 1) and component 2) depends from the properties of the active substances used, usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1, and the weight ratio of component 1) and component 3) usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1.

Any further active components are, if desired, added in a ratio of 20:1 to 1:20 to the component 1).

These ratios are also suitable for inventive mixtures applied by seed treatment.

The present invention also relates to a process for preparing the compound of the present invention. The process for preparing the compound of the present invention is described in the experimental section in more detail.

The invention disclosed in the present invention shall now be elaborated with the help of non-limiting schemes and examples.

Chemistry Schemes:

General Scheme:—

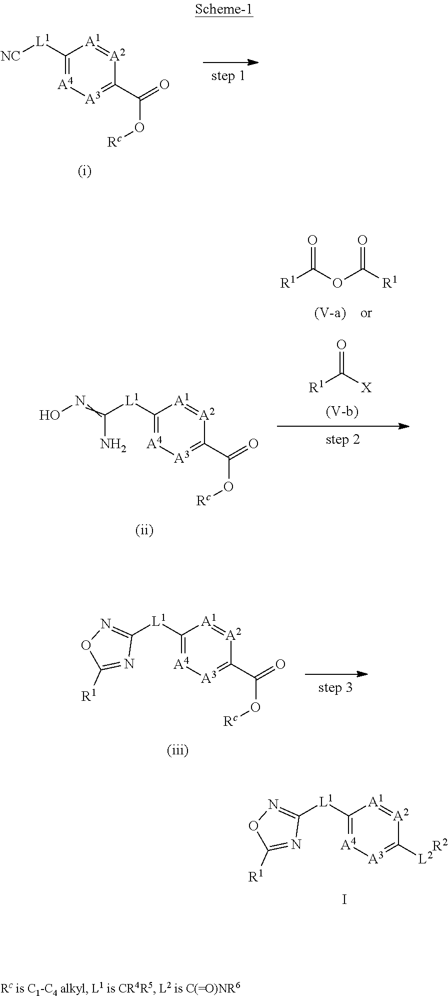

Step 1

A nitrile derivative of Formula (i) can be treated with hydroxylamine hydrochloride in the presence of a base such as sodium bicarbonate to provide a hydroxy imidamide derivative of Formula (ii). The reaction can also be carried out in the presence of aqueous solution of hydroxyl amine. The reaction can be typically carried out in solvents such as methanol, ethanol or tetrahydrofuran at 25-65° C.

Step 2

A compound of Formula (iii) is a critical intermediate and can be prepared by reacting the compound of Formula (ii) with acid anhydride of Formula (V-a). The reaction can be carried out in solvents such as tetrahydrofuran at 0-25° C.

The reaction can also be carried out by reacting the compound of Formula (ii) with acid halide (X═C or Br) optionally in the presence of an organic base such as triethyl amine, diisopropyl ethyl amine or pyridine. The reaction can be carried out in solvents such as tetrahydrofuran at 0-70° C.

Step 3

A compound of Formula I, wherein L² is C(═O)NR⁶ and R² and R⁶ are as defined in the detailed description, can be obtained by reacting the compound of Formula (iii) with amine in the presence of trimethyl aluminium. The reaction can be typically carried out in solvents such as toluene or tetrahydrofuran at 0-100° C.

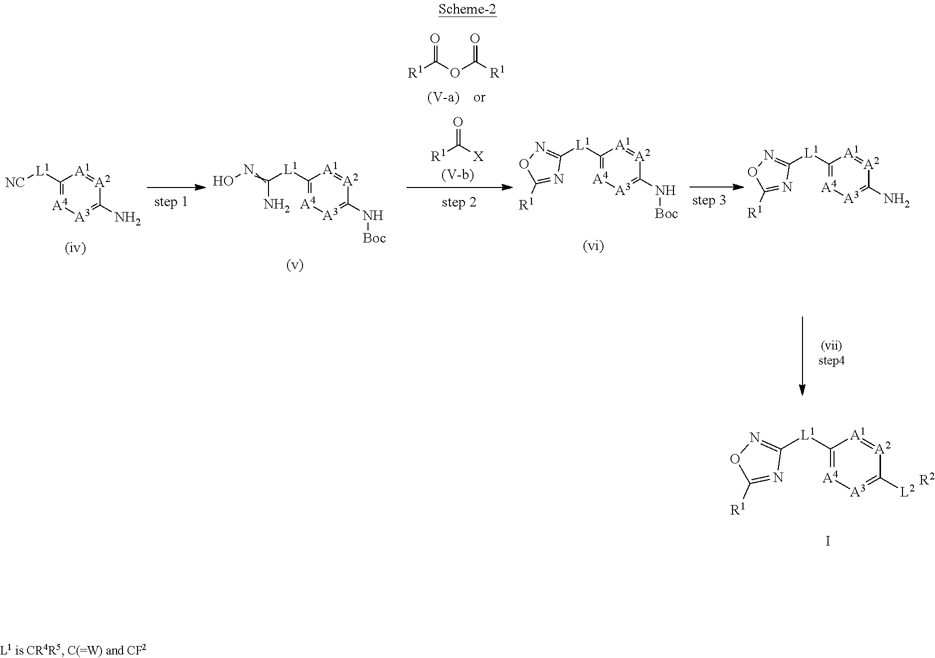

Step 1

A nitrile derivative of Formula (iv) can be treated with hydroxylamine hydrochloride in the presence of a base such as sodium bicarbonate to provide the hydroxy imidamide derivative of Formula (v). The reaction can be carried out in the presence of aqueous solution of hydroxyl amine. The reaction can be typically carried out in solvents such as methanol, ethanol or tetrahydrofuran at 25-65° C.

Step 2

A compound of Formula (vi) can be prepared by reacting compound of Formula (v) with the acid anhydride of Formula (V-a). The reaction can be carried out in solvents such as tetrahydrofuran at 0-25° C.

The reaction can also be carried out by reacting compound of Formula (v) with acid halide (X═Cl or Br) optionally in the presence of an organic base such as triethyl amine, diisopropyl ethyl amine or pyridine in solvents such as tetrahydrofuran at 0-70° C.

Step 3

The compound of Formula (vi) can be deprotected using acids such as hydrochloric acid or trifluoroacetic acid to obtain the respective salt of compound of Formula (vii).

The reaction can be typically carried out in solvents such as dichloromethane, tetrahydrofuran, 1,4-dioxane or diethyl ether at 0-40° C. The respective acid salt of compound of Formula (vii) can be reacted with aqueous solution of base such as sodium bicarbonate in solvents such as dichloromethane to obtain free amine compound of Formula (vii) at 5-25° C.

Step 4

The compound of Formula I wherein L² is NR⁶C(═O) and R² and R⁶ are as defined in the detailed description can be obtained by reacting an amine compound of Formula (vii) or its respective acid salt with acid chlorides in the presence of base such as triethyl amine, diisopropylethylamine or pyridine. The reaction can be carried out in solvents such as dichloromethane, tetrahydrofuran or toluene at 0-35° C.

Alternatively, the compound of Formula I, wherein L² is NR⁶C(═O) and R² and R⁶ are as defined in the detailed description, can be obtained by reacting the amine compound of Formula (vii) or its respective acid salt with organic acids in the presence of coupling reagents such as n-(3-Dimethylaminopropyl)-N′-ethylcarbodiimide hydrochloride, 1-Hydroxybenzotriazole or 1-[Bis(dimethylamino)methylene]-1H-1,2,3-triazolo[4,5-b]pyridinium 3-oxid hexafluorophosphate. The reaction can be typically carried out in the presence of organic bases such as triethyl amine or diisopropylethylamine in solvents such as dichloromethane, tetrahydrofuran, dimethylformamide or toluene at 0-35° C.

The compound of Formula I, wherein L² is NR⁶ and R² is C₁-C₆-haloalkylsulfonyl, arylsulfonyl, heteroarylsulfonyl, C₃-C₈-cycloalkylsulfonyl C₁-C₆-alkylsulfinyl, or C₁-C₆-alkylsulfonyl, can be obtained by reacting the amine compound of Formula (vii) or its respective acid salt with sulphonyl chlorides in the presence of base such as triethyl amine, diisopropylethylamine or pyridine. The reaction can be carried out in solvents such as dichloromethane, tetrahydrofuran or toluene at 0-35° C.

The compound of Formula I, wherein L² is NR⁶C(═O) and R² is C₁-C₆-alkoxy, aryloxy, heteroaryloxy, C₃-C₈-heterocylyloxy, C₃-C₈-cycloalkyloxy or C₁-C₆-haloalkoxy, can be obtained by reacting amine compound of Formula (vii) or its respective acid salt with the mentioned respective hydroxy compound in the presence of 1,1′-Carbonyldiimidazole, triphosgene or diphosgene. The reaction can be typically carried out in solvents such as dichloromethane, toluene, acetonitrile, tetrahydrofuran or dimethylformamide at 0-50° C. optionally in the presence of base such as triethyl amine, diisopropylethylamine or pyridine. Alternatively, the compound of Formula I can also be obtained by reacting the compound of Formula (vii) with respective chloroformates in the presence of a base such as triethylamine or diisopropylethylamine.

The compound of Formula I, wherein L² is NR⁶C(═O) and R² is C₁-C₆-alkylamino, arylamino, heteroarylamino, C₄-C₈-heterocyclylamino, C₁-C₆-dialkylamino, C₃-C₈-cycloalkylamino or C₁-C₆-alkyl-C₃-C₈-cycloalkylamino, can be obtained by reacting the amine compound of Formula (vii) or its respective acid salt with the mentioned respective amine in the presence of 1,1′-Carbonyldiimidazole, triphosgene or diphosgene. The reaction can be typically carried out in solvents such as dichloromethane, toluene, acetonitrile, tetrahydrofuran or dimethylformamide at 0-50° C. optionally in the presence of a base such as triethyl amine, diisopropylethylamine or pyridine. Alternatively, the compound of Formula I can also be obtained by reacting the compound of Formula (vii) with respective isocyanates in the presence of a base such as triethylamine or diisopropylethylamine.

Step 5:

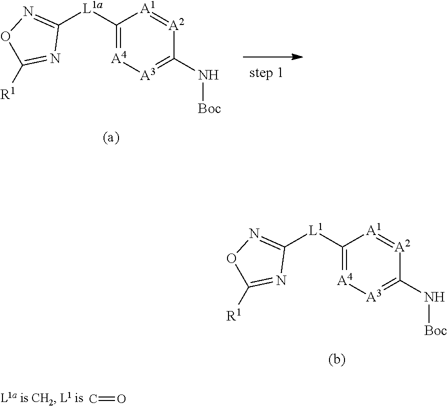

A compound of Formula (b) can be prepared by reacting a compound of Formula (a) first with oxidizing reagent such as tert-butylhydroperoxide in the presence of a catalyst such as palladium acetate (II) to obtain peroxide intermediate. The reaction can be carried out in organic protic solvents such as tert-butanol at 25-50° C. The obtained peroxide intermediate then reacted with a base such as triethylamine or diisopropylethylamine in solvents such as dichloromethane at 0-25° C. to obtain the compound of Formula (b).

Step 6:

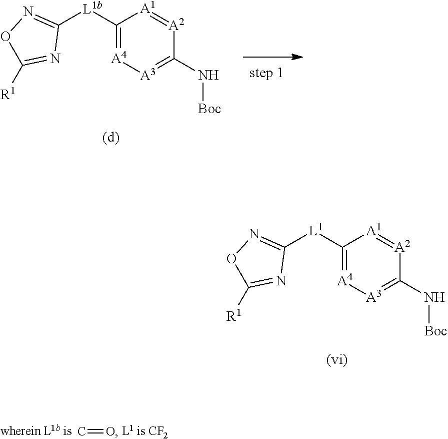

A compound of Formula (vi) can be prepared by reacting ketone a compound of Formula (d) with fluorinating reagent such as diethylamino sulfur trifluoride. The reaction can be carried out in solvents such as dichloromethane at 0-25° C.

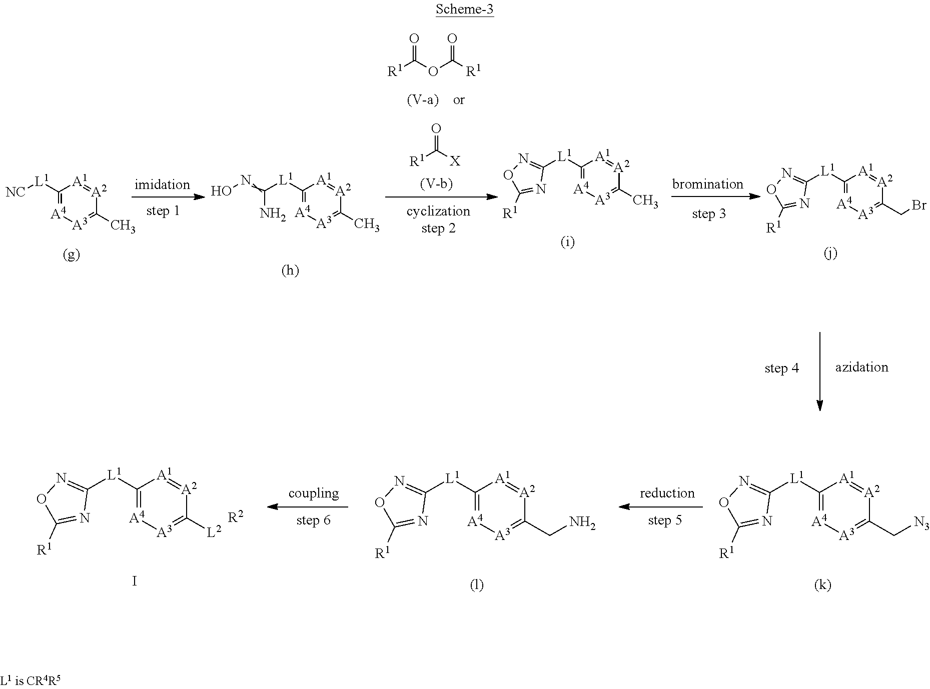

Step 1:

A nitrile derivative of Formula (g) can be treated with hydroxylamine hydrochloride in the presence of a base such as sodium bicarbonate to provide the hydroxy imidamide derivative of Formula (h). The reaction can be carried out in the presence of aqueous solution of hydroxyl amine. The reaction can be typically carried out in solvents such as methanol, ethanol or tetrahydrofuran at 25-65° C.

Step 2:

A compound of Formula (i) can be prepared by reacting compound of Formula (h) with an acid anhydride of Formula (V-a). The reaction can be carried out in solvents such as tetrahydrofuran at 0-25° C.

The reaction can also be carried out by reacting the compound of Formula (i) with acid halide (X═Cl or Br) in the presence of an organic base such as triethyl amine, diisopropyl ethyl amine or pyridine. The reaction can be carried out in solvents such as tetrahydrofuran at 0-70° C.

Step 3:

A compound of Formula (j) can be prepared by reacting the compound of Formula (i) with brominating reagents such as N-bromosuccinimide by radical bromination. The reaction can be carried out in the presence of radical initiator such as AIBN in aprotic solvents such as chloroform or tetrachloromethane at 0-50° C.

Step 4:

Azide compound of Formula (k) can be obtained by reacting bromo compound of Formula (j) with metal azides such as sodium azide. The reaction can be carried out in organic polar aprotic solvents such as DMF, DMSO or acetonitrile at 20-50° C.

Step 5:

Amino compound of Formula (l) can be prepared by Staudinger reaction of azide compound of Formula (k). The reaction can be carried out in the presence of reagents such as triphenylphosphine. The reaction is typically carried out in mixture of solvents such as tetrahydrofuran and water or 1,4-dioxane and water at 0-70° C.

Step 6:

The compound of Formula I, wherein L² is CR⁴R⁵ and R² is C₁-C₆-alkylcarbonylamino, C₃-C₆-cycloalkylcarbonylamino, arylcarbonylamino, heteroarylcarbonylamino, heterocyclylcarbonylamino or C₁₋C₆-haloalkylcarbonylamino, can be obtained by reacting the compound of Formula (l) with acid chlorides in the presence of base such as triethyl amine, diisopropylethylamine or pyridine. The reaction can be carried out in solvents such as dichloromethane, tetrahydrofuran or toluene at 0-35° C.

Alternatively, the compound of Formula I wherein L² is CR⁴R⁵; R² is C₁-C₆-alkylcarbonylamino, C₃-C₆-cycloalkylcarbonylamino, arylcarbonylamino, heteroarylcarbonylamino, heterocyclylcarbonylamino or C₁₋C₆-haloalkylcarbonylamino can also be prepared by reacting the amino compound of Formula (l) with organic acids in the presence of coupling reagents such as n-(3-Dimethylaminopropyl)-N′-ethylcarbodiimide hydrochloride, 1-Hydroxybenzotriazole or 1-[Bis(dimethylamino)methylene]-1H-1,2,3-triazolo[4,5-b]pyridinium 3-oxid hexafluorophosphate. The reaction can be typically carried out in the presence of organic bases such as triethyl amine or diisopropylethylamine in solvents such as dichloromethane, tetrahydrofuran, dimethylformamide or toluene at 0-35° C.

The compound of Formula I, wherein L² is CR⁴R⁵ and R² is sulfonamide, can be prepared by reacting the amino compound of Formula (l) with sulphonyl chlorides. The reaction can be carried out in the presence of base such as triethyl amine, diisopropylethylamine or pyridine. The reaction can be carried out in solvents such as dichloromethane, tetrahydrofuran or toluene at 0-35° C.

The compound of Formula I, wherein L² is CR⁴R⁵ and R² is C₁-C₆-alkylaminocarbonylamino, C₁-C₆-dialkylaminocarbonylamino, arylaminocarbonylamino, heteroarylaminocarbonylamino or C3-C6-cycloalkylaminocarbonylamino, can be prepared by reacting the compound of Formula (l) with isocynates. The reaction can be carried out in solvents such as dichloromethane, tetrahydrofuran or acetonitrile.

The compound of Formula I, wherein L² is CR⁴R⁵ and R² is C₁-C₆-alkyloxycarbonylamino, aryloxycarbonylamino, heterocycloxycarbonylamino, heteroaryloxycarbonylamino or C3-C6-cycloalkyloxycarbonylamino, can be prepared by reacting the amino compound of Formula (l) with chloroformates. The reaction can be carried out in the presence of base such as triethylamine triethyl amine, diisopropylethylamine or pyridine. The reaction can be carried out in solvents such as dichloromethane, tetrahydrofuran or toluene at 0-35° C.

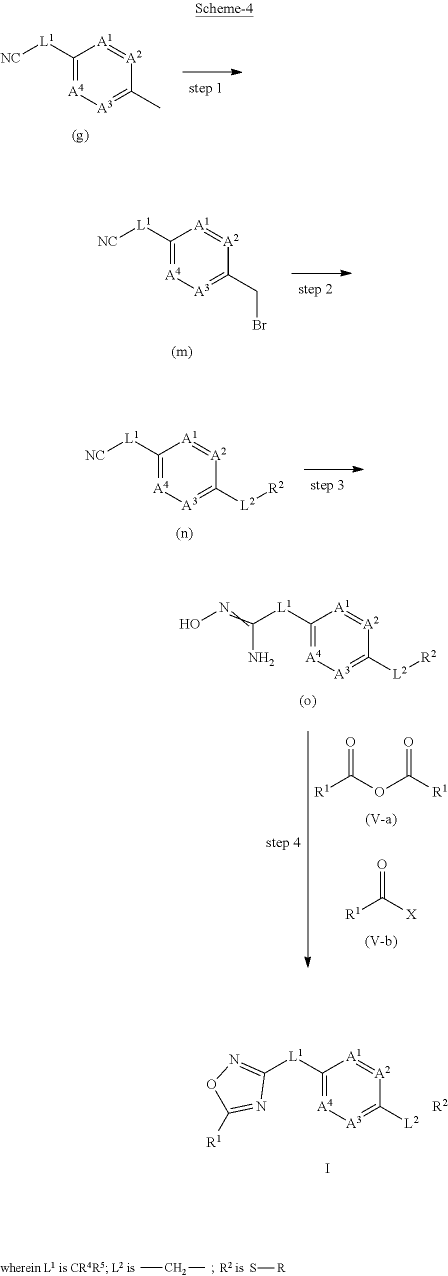

Step 1:

A bromo compound of Formula (m) can be prepared by reacting compound of Formula (g) with brominating reagents such as N-bromosuccinimide by radical bromination. The reaction can be carried out in the presence of radical initiator such as AIBN in aprotic solvents such as chloroform or tetrachloromethane at 0-50° C.

Step 2:

A compound of Formula (n), wherein L² is CR⁴R⁵ and R² is C₁-C₆-alkylthio, arylthio, heteroarylthio, C₄-C₅-heterocyclylthio or C₁-C₆-haloalkylthio, can be obtained by reacting a bromo compound of Formula (m) with mercapto compound. The reaction can be carried out in the presence of a base such as potassium tert-butoxide or sodium tert-butoxide. The reaction can be carried out in organic polar aprotic solvents such as N,N-dimethylformamide at 0-35° C.

Step 3:

The nitrile derivative (n) is treated with hydroxylamine hydrochloride in the presence of a base such as sodium bicarbonate to provide a hydroxy imidamide derivative of Formula (o). The reaction can also be carried out in the presence of aqueous solution of hydroxyl amine. The reaction can be typically carried out in solvents such as methanol, ethanol or tetrahydrofuran at 25-65° C.

Step 4:

The compound of Formula I, wherein L² is CR⁴R⁵ and R² is C₁-C₆-alkylthio, arylthio, heteroarylthio, C₄-C₅-heterocyclylthio or C₁-C₆-haloalkylthio, can be prepared by reacting the compound of Formula (o) with acid anhydride of Formula (V-a). The reaction can be carried out in solvent such as tetrahydrofuran at 0-25° C.

The reaction can also be carried out by reacting the compound of Formula (o) with acid halide (V-b) (X═Cl or Br) optionally in the presence of an organic base such as triethyl amine, diisopropyl ethyl amine or pyridine. The reaction can be carried out in solvents such as tetrahydrofuran at 0-70° C.

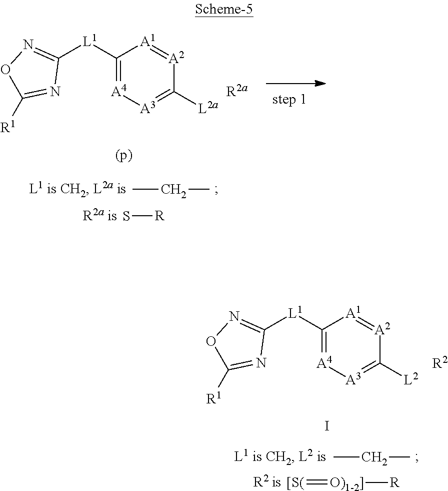

The compound of Formula I, wherein L¹ is CH₂, L^{2a} is CR⁴R⁵ and R² is C₁-C₆-haloalkylsulfinyl, arylsulfinyl, heteroarylsulfinyl, C₃-C₈-cycloalkylsulfinyl, C₁-C₆-alkylsulfinyl, C₁-C₆-haloalkylsulfonyl, arylsulfonyl, heteroarylsulfonyl, C₃-C₈-cycloalkylsulfonyl or C₁-C₆-alkylsulfonyl, can be prepared by reacting a compound of Formula (p), wherein L¹ is CH₂, L² is CR⁴R⁵ and R² is C₁-C₆-alkylthio, arylthio, heteroarylthio, C₄-C₅-heterocyclylthio or C₁-C₆-haloalkylthio, with oxidizing reagents such as m-CPBA or oxone. The reaction is carried out in solvent such as dichloromethane at 0-25° C.

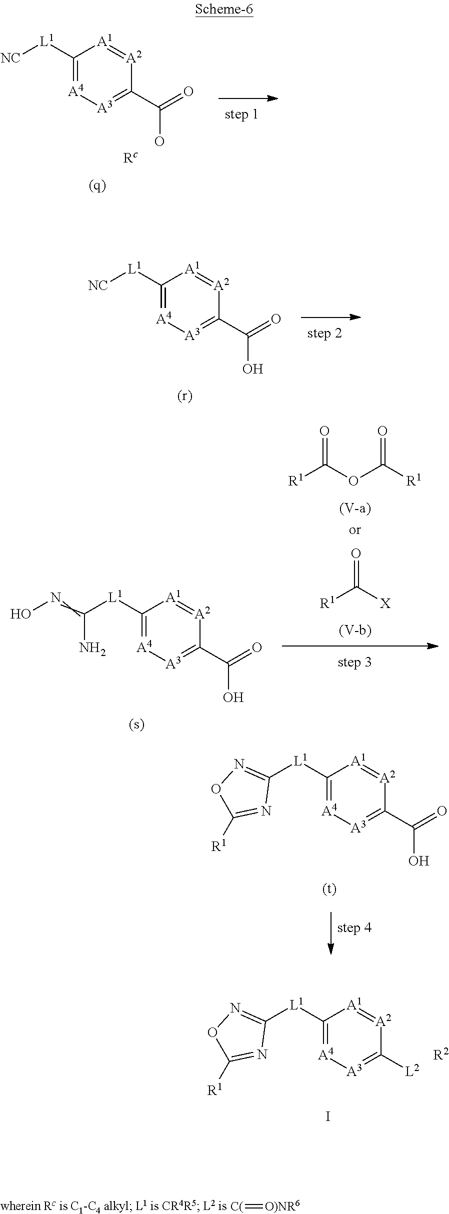

Step 1:

A compound of Formula (r), wherein L¹ is CR⁴R⁵, can be obtained by reacting a compound of Formula (q), wherein R^{c} is C₁_4 alkyl, with an alkylating reactant such as haloalknes (methyl iodide or 1,2-dibromoethane) in the presence of metal hydride base such as sodium hydride. The reaction condition, ester hydrolysis also takes place. The reaction can be typically carried out in organic polar aprotic solvents such as tetrahydrofuran or N,N-dimethyl formamide at 0-25° C.

Step 2:

A nitrile derivative of Formula (r), wherein L¹ is CR⁴R⁵, is treated with hydroxylamine hydrochloride in the presence of a base such as sodium bicarbonate to provide the hydroxy imidamide derivative of Formula (s). The reaction can also be carried out in the presence of aqueous solution of hydroxyl amine. The reaction can be typically carried out in solvents such as methanol, ethanol or tetrahydrofuran at 25-65° C.

Step 3:

A compound of Formula (t) can be prepared by reacting the compound of Formula (s) with an acid anhydride of Formula (V-a). The reaction can be carried out in solvent such as tetrahydrofuran at 0-25° C.

Step 4:

The compound of Formula I, wherein L² is C(═O)NR⁶ and R² is as defined in the detailed description, can be obtained by reacting the compound of Formula (t) with amino compound. The reaction can be carried out in the presence of coupling reagents such as n-(3-Dimethylaminopropyl)-N′-ethylcarbodiimide hydrochloride, 1-Hydroxybenzotriazole or 1-[Bis(dimethylamino)methylene]-1H-1,2,3-triazolo[4,5-b]pyridinium 3-oxid hexafluorophosphate. The reaction can be typically carried out in the presence of organic bases such as triethyl amine or diisopropylethylamine in solvents such as dichloromethane, tetrahydrofuran, N,N-dimethylformamide or toluene at 0-35° C.

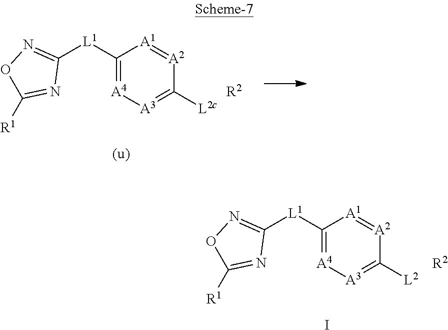

The compound of Formula I, wherein, L¹ is CR⁴R⁵, L² is C(═S) and R² is C₁-C₆-alkylamino, arylamino, heteroarylamino, C₄-C₈-heterocyclylamino, C₁-C₆-dialkylamino, C₃-C₈-cycloalkylamino or C₁-C₆-alkyl-C₃-C₈-cycloalkylamino, can be prepared by reacting a compound of Formula (s) wherein L¹ is CR⁴R⁵, L^{2c} is C(═O) and R² is C₁-C₆-alkylamino, arylamino, heteroarylamino, C₄-C₈-heterocyclylamino, C₁-C₆-dialkylamino, C₃-C₈-cycloalkylamino or C₁-C₆-alkyl-C₃-C₈-cycloalkylamino, with 2,4-bis(4-methoxyphenyl)-1,3,2,4-dithiadiphosphetane-2,4-disulfide (Lawesson's reagent). The reaction can be carried out in solvents such as tetrahydrofuran or 1,4-dioxane at 0-80° C.

### CHEMISTRY EXAMPLES

### Example 1:—Preparation of 4-methyl-N-(4-((5-(trifluoromethyl)-1,2,4-oxadiazol-3-yl)methyl)phenyl)benzamide (Compound 1)

### Method A

### Step-1:—Preparation of tert-butyl (4-(cyanomethyl)phenyl)carbamate

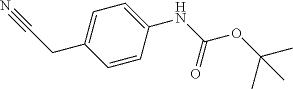 [SMILES: CC(C)(C)OC(=O)Nc1ccc(CC#N)cc1]

To a solution of 2-(4-aminophenyl)acetonitrile (5 g, 38 mmol) in ethanol (50 mL), di-tert-butyl dicarbonate (26 mL, 113 mmol) was added at 0-5° C. The resulting reaction mixture was stirred for 24 h at 25° C. The reaction mixture was concentrated and the residue was stirred with 50 mL of hexanes for 30 min and filtered. The obtained solid was washed again with hexanes (20 mL) and dried under reduced pressure to obtain tert-butyl (4-(cyanomethyl)phenyl)carbamate (8 g, 34 mmol, 91% yield).

### Step 2:—Preparation of 2 tert-butyl (4-(2-amino-2-(hydroxyimino)ethyl)phenyl)carbamate

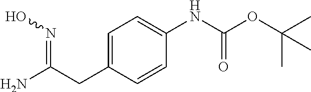

To a solution of tert-butyl (4-(cyanomethyl)phenyl)carbamate (5 g, 22 mmol) in methanol (50 mL), 50% aqueous solution of hydroxylamine (4.7 mL, 86 mmol) was added and stirred for 24 h at 60° C. The volatiles were evaporated under reduced pressure. The residue was triturated with toluene (50 mL) and filtered. The obtained solid was washed with hexanes (20 mL) and dried under reduced pressure to obtain tert-butyl (4-(2-amino-2-(hydroxyimino)ethyl)phenyl)carbamate (5.2 g, 20 mmol, 91% yield).

### Step 3:—Preparation of tert-butyl (4-((5-(trifluoromethyl)-1,2,4-oxadiazol-3-yl)methyl)phenyl)carbamate

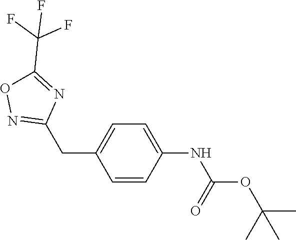 [SMILES: CC(C)(C)OC(=O)Nc1ccc(Cc2noc(C(F)(F)F)n2)cc1]

To a solution of tert-butyl (4-(2-amino-2-(hydroxyimino)ethyl)phenyl)carbamate (5 g, 18.8 mmol) in tetrahydrofuran (50 mL), 2,2,2-trifluoroacetic anhydride (4.7 mL, 34 mmol) was added at 0-5° C. and stirred for 24 h. The resulting reaction mixture was poured into saturated sodium carbonate solution at 0-5° C. and then diluted with 100 mL of dichloromethane. The dichloromethane layer was separated, washed with water (50 mL) and brine solution (50 mL) and then dried over anhydrous sodium sulphate and concentrated. The crude product was purified on column chromatography to obtain pure tert-butyl (4-((5-(trifluoromethyl)-1,2,4-oxadiazol-3-yl)methyl)phenyl)carbamate (5.1 g, 15 mmol, 79% yield).

### Step 4:—Preparation of 4-((5-(trifluoromethyl)-1,2,4-oxadiazol-3-yl)methyl)aniline-

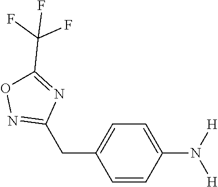 [SMILES: Nc1ccc(Cc2noc(C(F)(F)F)n2)cc1]

To a solution of tert-butyl (4-((5-(trifluoromethyl)-1,2,4-oxadiazol-3-yl)methyl)phenyl)carbamate (0.5 g, 1.5 mmol) in dichloromethane (5.5 mL), trifluoroacetic acid (1.35 mL, 17.5 mmol) was added at 0-5° C. and stirred at 25° C. for 3 h. After completion of the reaction, the reaction mixture was poured into saturated sodium carbonate solution (10 mL) at 0-5° C. The aqueous layer was extracted thrice with dichloromethane (50 mL). The combined dichloromethane layer was washed with water (50 mL), brine solution (50 mL), dried over anhydrous sodium sulphate and evaporated under reduced pressure to obtain crude product which was purified on column chromatography to obtain pure 4-((5-(trifluoromethyl)-1,2,4-oxadiazol-3-yl)methyl)aniline (0.32 g, 1.5 mmol, 90% yield).

### Step 5:—Preparation of 4-methyl-N-(4-((5-(trifluoromethyl)-1,2,4-oxadiazol-3-yl)methyl)phenyl)benzamide (Compound 1)

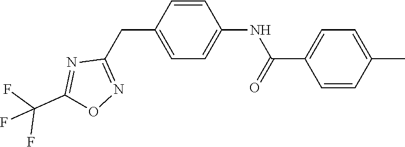 [SMILES: Cc1ccc(C(=O)Nc2ccc(Cc3noc(C(F)(F)F)n3)cc2)cc1]

To a solution 4-methylbenzoic acid (140 mg, 1.03 mmol) in dichloromethane (3 mL), N-ethyl-N-isopropylpropan-2-amine (0.37 mL, 2.06 mmol) and 2-(3H-[1,2,3]triazolo[4,5-b]pyridin-3-yl)-1,1,3,3-tetramethylisouronium hexafluorophosphate(V) (391 mg, 1.03 mmol) were added and stirred for 30 min. 4-((5-(trifluoromethyl)-1,2,4-oxadiazol-3-yl)methyl)aniline (250 mg, 1.03 mmol) was added and stirred for 16 h at 25° C. The resulting reaction mixture was quenched with water (5 mL), extracted twice with dichloromethane (10 ml). The dichloromethane layer was washed with water (5 mL), brine solution (5 mL), dried over anhydrous sodium sulphate and concentrated under reduced pressure at 50° C. The crude compound was purified on column chromatography to obtain pure 4-methyl-N-(4-((5-(trifluoromethyl)-1,2,4-oxadiazol-3-yl)methyl)phenyl)benzamide (0.34 g, 0.94 mmol, 92% yield). 1H-NMR (400 MHz, DMSO-D6) δ 10.16 (s, 1H), 7.85 (d, 2H), 7.72-7.74 (m, 2H), 7.28-7.33 (m, 4H), 4.23 (s, 2H), 2.37 (s, 3H); (M+1): 362.20

### Method B—Preparation of 2-phenyl-N-(4-((5-(trifluoromethyl)-1,2,4-oxadiazol-3-yl)methyl)phenyl)acetamide (compound 6)

### Step-1:—Preparation of 2-phenyl-N-(4-((5-(trifluoromethyl)-1,2,4-oxadiazol-3-yl)methyl)phenyl)acetamide (compound 6)

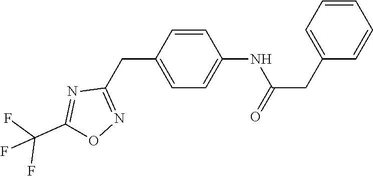 [SMILES: O=C(Cc1ccccc1)Nc1ccc(Cc2noc(C(F)(F)F)n2)cc1]

To a solution of 4-((5-(trifluoromethyl)-1,2,4-oxadiazol-3-yl)methyl)aniline (250 mg, 1.03 mmol) in dichloromethane (2.5 mL), diisopropylethylamine (0.18 mL, 1.03 mmol), 4-dimethylaminopyridine (12.6 mg, 0.10 mmol) and 2-phenylacetyl chloride (0.15 mL, 1.13 mmol) were added at 0-5° C. The resulting reaction mixture was then stirred for 3 h at 25° C. After completion of the reaction, the reaction mixture was cooled to 25° C. and cautiously basified with sodium bicarbonate to pH 7-8. The aqueous layer was extracted thrice with dichloromethane (25 mL). The combined dichloromethane layer was washed with water (25 mL), brine solution (25 mL), dried over anhydrous sodium sulphate and concentrated. The obtained crude product was purified on coulmn chromatography to obtain pure 2-phenyl-N-(4-((5-(trifluoromethyl)-1,2,4-oxadiazol-3-yl)methyl)phenyl)acetamide (0.34 g, 1.03 mmol, 92% yield).

1H-NMR (400 MHz, DMSO-D6) δ 10.17 (s, 1H), 7.55 (d, 2H), 7.30-7.31 (m, 4H), 7.22-7.26 (m, 3H), 4.19 (s, 2H), 3.61 (s, 2H); (M+1): 362.05

TABLE 1
The following compounds were prepared by the procedure
analogous to that for the Compound No. 1 or 6

| Compound | | | | |
|---|---|---|---|---|
| No. | IUPAC Name | NMR and LCMS data | Method | Yield |
| 2 | N-(4-((5- | ¹H-NMR (400 MHz, DMSO-D6) δ | A | 0.41 g, |
| | (trifluoromethyl)- | 10.25 (s, 1H), 7.92-7.94 (m, 2H), | | 96% yield |
| | 1,2,4-oxadiazol-3- | 7.74 (d, 2H), 7.56-7.60 (m, 1H), | | |
| | yl)methyl)phenyl)benz- | 7.50-7.54 (m, 2H), 7.31 (d, 2H), 4.23 | | |
| | amide | (s, 2H); (M + 1): 348.05 | | |
| 3 | N-(4-((5- | ¹H-NMR (400 MHz, DMSO-D6) δ | A | 0.34 g, |
| | (trifluoromethyl)- | 10.65 (s, 1H), 8.73 (dq, 1H), 8.15 | | 95% yield |
| | 1,2,4-oxadiazol-3- | (dt, 1H), 8.06 (td, 1H), 7.85-7.88 | | |
| | yl)methyl)phenyl)pico- | (m, 2H), 7.65-7.68 (m, 1H), 7.32 (d, | | |
| | linamide | 2H), 4.24 (s, 2H); (M + 1): 349.00 | | |
| 4 | N-(4-((5- | ¹H-NMR (400 MHz, DMSO-D6) δ | A | 0.31 g, |
| | (trifluoromethyl)- | 10.44 (s, 1H), 9.07-9.09 (m, 1H), | | 87% yield |
| | 1,2,4-oxadiazol-3- | 8.75 (dd, 1H), 8.26-8.29 (m, 1H), | | |
| | yl)methyl)phenyl)nico- | 7.72-7.74 (m, 2H), 7.56 (ddd, 1H), | | |
| | tinamide | 7.33 (d, 2H), 4.24 (s, 2H); (M + 1): | | |
| | | 348.60 | | |
| 5 | N-(4-((5- | ¹H-NMR (400 MHz, DMSO-D6) δ | A | 0.29 g, |
| | (trifluoromethyl)- | 10.50 (s, 1H), 8.76-8.78 (m, 2H), | | 82% yield |
| | 1,2,4-oxadiazol-3- | 7.83-8.85 (m, 2H), 7.73 (d, 2H), 7.33 | | |
| | yl)methyl)phenyl)iso- | (d, 2H), 4.24 (s, 2H); (M + 1): 349.00 | | |
| | nicotinamide | | | |
| 7 | 4-cyano-N-(4-((5- | ¹H-NMR (400 MHz, DMSO-D6) δ | A | 0.32 g, |
| | (trifluoromethyl)- | 10.49 (s, 1H), 8.07-8.09 (m, 2H), | | 84% yield |
| | 1,2,4-oxadiazol-3- | 8.00-8.03 (m, 2H), 7.73 (d, 2H), 7.33 | | |
| | yl)methyl)phenyl)benz- | (d, 2H), 4.24 (s, 2H); (M − 1): 370.95 | | |
| | amide | | | |
| 8 | 4-(trifluoromethyl)- | ¹H-NMR (400 MHz, DMSO-D6) δ | B | 0.34 g, |
| | N-(4-((5- | 10.47 (s, 1H), 8.13 (d, 2H), 7.91 (d, | | 81% yield |
| | (trifluoromethyl)- | 2H), 7.74 (d, 2H), 7.33 (d, 2H), 4.24 | | |
| | 1,2,4-oxadiazol-3- | (s, 2H); (M + 1): 416.00 | | |
| | yl)methyl)phenyl)benz- | | | |
| | amide | | | |
| 9 | 4-fluoro-N-(4-((5- | ¹H-NMR (400 MHz, DMSO-D6) δ | B | 0.34 g, |
| | (trifluoromethyl)- | 10.27 (s, 1H), 7.99-8.03 (m, 2H), | | 92% yield |
| | 1,2,4-oxadiazol-3- | 7.71-7.73 (m, 2H), 7.34-7.39 (m, | | |
| | yl)methyl)phenyl)benz- | 2H), 7.31 (d, 2H), 4.23 (s, 2H); | | |
| | amide | (M + 1): 366.00 | | |
| 10 | 4-chloro-N-(4-((5- | ¹H-NMR (400 MHz, DMSO-D6) δ | A | 0.32 g, |
| | (trifluoromethyl)- | 10.32 (s, 1H), 7.95-7.98 (m, 2H), | | 82% yield |
| | 1,2,4-oxadiazol-3- | 7.72 (d, 2H), 7.59-7.61 (m, 2H), 7.31 | | |
| | yl)methyl)phenyl)benz- | (d, 2H), 4.23 (s, 2H); (M + 1): 382.00 | | |
| | amide | | | |
| 11 | 2-(4-fluorophenyl)- | ¹H-NMR (400 MHz, DMSO-D6) δ | A | 0.30 g, |
| | N-(4-((5- | 10.17 (s, 1H), 7.54 (d, 2H), 7.31- | | 75% yield |
| | (trifluoromethyl)- | 7.35 (m, 2H), 7.24 (d, 2H), 7.11-7.16 | | |
| | 1,2,4-oxadiazol-3- | (m, 2H), 4.19 (s, 2H), 3.61 (s, 2H); | | |
| | yl)methyl)phenyl)acet- | (M + 1): 380.05 | | |
| | amide | | | |

### Example 2:—Preparation of 1-isopropyl-3-(4-((5-(trifluoromethyl)-1,2,4-oxadiazol-3-yl)methyl)phenyl)urea. (Compound No. 73)

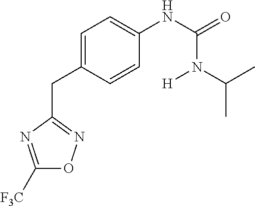 [SMILES: CC(C)NC(=O)Nc1ccc(Cc2noc(C(F)(F)F)n2)cc1]

To a stirred solution of 4-((5-(trifluoromethyl)-1,2,4-oxadiazol-3-yl)methyl)aniline (250 mg, 1 mmol) in acetonitrile (5 mL), triethyl amine (0.3 mL, 2.1 mmol) was added under nitrogen atmosphere at 0° C., 2-isocyanatopropane (0.1 mL, 1.3 mmol) was added after stirring for 10 min. The resulting reaction mixture was stirred at 25° C. for 16 h. After completion of the reaction, the reaction mixture was filtered to obtain solid product 1-isopropyl-3-(4-((5-(trifluoromethyl)-1,2,4-oxadiazol-3-yl)methyl)phenyl)urea (159 mg, 0.5 mmol, 47% yield).

1H-NMR (400 MHz, DMSO-D6) δ 8.27 (s, 1H), 7.33-7.30 (m, 2H), 7.17-7.13 (i, 2H), 5.95 (d, 1H), 4.14 (s, 2H), 3.72 (dt, 1H), 1.09-1.06 (s, 6H); LCMS (M+H): 329

TABLE 2
The following compounds were prepared by the procedure
analogous to that for the Compound No. 73

| Compound | | | |
|---|---|---|---|
| No. | IUPAC Name | NMR and LCMS data | Yield |
| 74 | 1-(pyridin-3-yl)-3-(4-((5- | 1H-NMR (400 MHz, DMSO-D6) δ 8.82 | 281 mg, |
| | (trifluoromethyl)-1,2,4- | (d, 2H), 8.58 (d, 1H), 8.17 (dd, 1H), 7.94- | 75% yield |
| | oxadiazol-3- | 7.90 (m, 1H), 7.42 (d, 2H), 7.30 (dd, 1H), | |
| | yl)methyl)phenyl)urea | 7.25 (d, 2H), 4.19 (s, 2H); LCMS (M + H): | |
| | | 364 | |
| 75 | 1-(4-methoxyphenyl)-3-(4- | 1H-NMR (400 MHz, DMSO-D6) δ 8.57 | 330 mg, |
| | ((5-(trifluoromethyl)-1,2,4- | (s, 1H), 8.43 (s, 1H), 7.40 (dd, 2H), 7.34- | 82% yield |
| | oxadiazol-3- | 7.31 (m, 2H), 7.22 (d, 2H), 6.87-6.84 (m, | |
| | yl)methyl)phenyl)urea | 2H), 4.18 (s, 2H), 3.70 (s, 3H); LCMS | |
| | | (M + H): 393 | |
| 76 | 1-(p-tolyl)-3-(4-((5- | 1H-NMR (400 MHz, DMSO-D6) δ 8.61 | 305 mg, |
| | (trifluoromethyl)-1,2,4- | (s, 1H), 8.52 (s, 1H), 7.41-7.39 (m, 2H), | 79% yield |
| | oxadiazol-3- | 7.31 (d, 2H), 7.22 (d, 2H), 7.06 (d, 2H), | |
| | yl)methyl)phenyl)urea | 4.18 (s, 2H), 2.23 (s, 3H); LCMS(M + H): | |
| | | 377 | |
| 77 | 1-(4-chlorophenyl)-3-(4-((5- | 1H-NMR (400 MHz, DMSO-D6) δ 8.78 | 300 mg, |
| | (trifluoromethyl)-1,2,4- | (s, 1H), 8.70 (s, 1H), 7.47-7.44 (m, 2H), | 74% yield |
| | oxadiazol-3- | 7.41 (dd, 2H), 7.32-7.29 (m, 2H), 7.24 (d, | |
| | yl)methyl)phenyl)urea | 2H), 4.19 (s, 2H); LCMS (M + H): 397 | |
| 78 | 1-(4-fluorophenyl)-3-(4-((5- | 1H-NMR (400 MHz, DMSO-D6) δ 8.66 | 151 mg, |
| | (trifluoromethyl)-1,2,4- | (d, 2H), 7.39-7.46 (m, 4H), 7.23 (d, 2H), | 39% |
| | oxadiazol-3- | 7.08-7.13 (m, 2H), 4.18 (s, 2H); | |
| | yl)methyl)phenyl)urea | LCMS(M + H): 381 | |
| 80 | 1-phenyl-3-(4-((5- | 1H-NMR (400 MHz, DMSO-D6) δ 8.64 | 153 mg, |
| | (trifluoromethyl)-1,2,4- | (d, 2H), 7.44-7.40 (m, 4H), 7.28-7.22 (m, | 41% yield |
| | oxadiazol-3- | 4H), 6.97-6.93 (m, 1H), 4.18 (s, 2H); | |
| | yl)methyl)phenyl)urea | LCMS (M + H): 363 | |
| 81 | 1-ethyl-3-(4-((5- | 1H-NMR (400 MHz, DMSO-D6) δ 8.39 | 105 mg, |
| | (trifluoromethyl)-1,2,4- | (s, 1H), 7.32-7.35 (m, 2H), 7.16 (d, 2H), | 32% yield |
| | oxadiazol-3- | 6.05 (t, 1H), 4.14 (s, 2H), 3.08 (td, 2H), | |
| | yl)methyl)phenyl)urea | 1.03 (d, 3H); LCMS(M + H): 315 | |
| 89 | 1-(cyclopropylmethyl)-3-(4- | 1H-NMR (400 MHz, DMSO-D6) δ 8.41 | 234 mg, |
| | ((5-(trifluoromethyl)-1,2,4- | (d, 1H), 7.35-7.32 (m, 2H), 7.17-7.15 (m, | 67% yield |
| | oxadiazol-3- | 2H), 6.16 (t, 1H), 4.14 (s, 2H), 2.94 (t, | |
| | yl)methyl)phenyl)urea | 2H), 0.93-0.87 (m, 1H), 0.43-0.38 (m, | |
| | | 2H), 0.18-0.14 (m, 2H): LCMS (M + H): | |
| | | 341.05 | |
| 90 | 1-(tert-butyl)-3-(4-((5- | 1H-NMR (400 MHz, DMSO-D6) δ 8.23 | 195 mg, |
| | (trifluoromethyl)-1,2,4- | (s, 1H), 7.33-7.30 (m, 2H), 7.17 (d, 2H), | 69% yield |
| | oxadiazol-3- | 5.97 (s, 1H), 4.16 (s, 2H), 1.28 (s, 9H); | |
| | yl)methyl)phenyl)urea | LCMS (M + H): 343 | |

### Example 3:—Preparation of phenyl (4-((5-(trifluoromethyl)-1,2,4-oxadiazol-3-yl)methyl)phenyl)carbamate. (Compound No. 91)

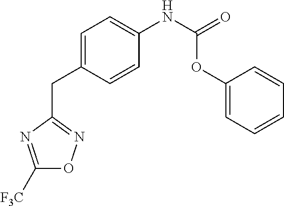 [SMILES: O=C(Nc1ccc(Cc2noc(C(F)(F)F)n2)cc1)Oc1ccccc1]

To a stirred solution of 4-((5-(trifluoromethyl)-1,2,4-oxadiazol-3-yl)methyl)aniline (200 mg, 0.8 mmol) in ethanol (5 mL), triethylamine (0.2 mL, 1.6 mmol) was added under nitrogen atmosphere at 0° C. and phenyl carbonochloridate (0.1 mL, 0.8 mmol) was added after stirring for 10 min. The resulting reaction mixture was stirred at 25° C. for 16 h. After completion of the reaction, the reaction mixture was filtered to obtain phenyl (4-((5-(trifluoromethyl)-1,2,4-oxadiazol-3-yl)methyl)phenyl)carbamate (79 mg, 0.2 mmol, 26% yield).

1H-NMR (400 MHz, DMSO-D6) δ 10.22 (s, 1H), 7.47 (d, 2H), 7.44-7.39 (m, 2H), 7.29-7.24 (m, 3H), 7.22-7.19 (m, 2H), 4.20 (s, 2H); LCMS (M): 363

TABLE 3
The following compound was prepared by the procedure
analogous to that for the Compound No. 91

| Compound | | | |
|---|---|---|---|
| No. | IUPAC Name | NMR and LCMS data | Yield |
| 92 | methyl (4-((5-(trifluoromethyl)- | 1H-NMR (400 MHz, DMSO-D6) | 166 mg, |
| | 1,2,4-oxadiazol-3- | δ 9.63 (d, 1H), 7.43-7.39 (m, 2H), | 67% yield |
| | yl)methyl)phenyl)carbamate | 7.24-7.21 (m, 2H), 4.17 (s, 2H), | |
| | | 3.64 (s, 3H); LCMS (M): 301 | |

### Example 4: Preparation of N-(4-((5-(trifluoromethyl)-1,2,4-oxadiazol-3-yl)methyl)phenyl)benzenesulfonamide (Compound No. 68)

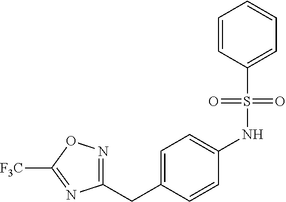 [SMILES: O=S(=O)(Nc1ccc(Cc2noc(C(F)(F)F)n2)cc1)c1ccccc1]

To a stirred solution of 4-((5-(trifluoromethyl)-1,2,4-oxadiazol-3-yl)methyl)aniline (0.2 g, 1.0 mmol) in dichloromethane (10 mL), triethylamine (0.6 mL, 4.2 mmol) was added followed by the addition of benzenesulfonyl chloride (0.1 mL, 0.6 mmol) at 0° C. The resulting reaction mixture was allowed to stir for 30 min at 25° C. After completion of the reaction, the reaction was quenched by saturated aqueous sodium bicarbonate solution and extracted with dichloromethane (30 mL). The dichloromethane layer was separated, dried over anhydrous sodium sulphate and evaporated under reduced pressure to obtain a crude compound which was purified by column chromatography to obtain N-(4-((5-(trifluoromethyl)-1,2,4-oxadiazol-3-yl)methyl)phenyl)benzenesulfonamide (0.2 g, 0.44 mmol, 42% yield).

1H-NMR (400 MHz, DMSO-D6) δ 10.33 (d, 1H), 7.78-7.76 (m, 2H), 7.62-7.53 (m, 3H), 7.21-7.19 (m, 2H), 7.08-7.05 (m, 2H), 4.15 (s, 2H); LCMS (M−H): 381.95

TABLE 4
The following compounds were prepared by the procedure
analogous to that for the compound No. 68

| Compound | | | |
|---|---|---|---|
| No. | IUPAC Name | NMR and LCMS data | Yield |
| 69 | 4-fluoro-N-(4-((5-(trifluoromethyl)- | 1H-NMR (400 MHz, DMSO-D6) δ | 130 mg, |
| | 1,2,4-oxadiazol-3- | 10.34 (d, 1H), 7.84-7.80 (m, 2H), | 31% yield |
| | yl)methyl)phenyl)benzenesulfonamide | 7.42-7.37 (m, 2H), 7.23-7.20 (m, | |
| | | 2H), 7.08-7.05 (m, 2H), 4.17 (s, | |
| | | 2H); LCMS (M−): 431.8 | |
| 70 | 4-methyl-N-(4-((5-(trifluoromethyl)- | 1H-NMR (400 MHz, DMSO-D6) δ | 107 mg, |
| | 1,2,4-oxadiazol-3- | 10.26 (s, 1H), 7.66-7.64 (m, 2H), | 26% yield |
| | yl)methyl)phenyl)benzenesulfonamide | 7.40-7.33 (m, 2H), 7.20-7.18 (m, | |
| | | 2H), 7.07-7.04 (m, 2H), 4.15 (s, | |
| | | 2H), 2.34 (s, 3H); LCMS (M − H): | |
| | | 396.15 | |
| 72 | 3-chloro-N-(4-((5-(trifluoromethyl)- | 1H-NMR (400 MHz, DMSO-D6) δ | 87 mg, |
| | 1,2,4-oxadiazol-3- | 10.42 (s, 1H), 7.74 (t, 1H), 7.72- | 20% yield |
| | yl)methyl)phenyl)benzenesulfonamide | 7.69 (m, 2H), 7.59 (t, 1H), 7.24 (d, | |
| | | 2H), 7.07 (dd, 2H), 4.18, (s, 2H); | |
| | | LCMS (M − H): 417.90 | |
| 79 | 2-fluoro-N-(4-((5-(trifluoromethyl)- | 1H-NMR (400 MHz, DMSO-D6) δ | 97 mg, |
| | 1,2,4-oxadiazol-3- | 10.65 (d, 1H), 7.83 (d, 1H), 7.72- | 23% yield |
| | yl)methyl)phenyl)benzenesulfonamide | 7.54 (m, 1H), 7.41-7.34 (m, 2H), | |
| | | 7.22-7.20 (m, 2H), 7.08 (dd, 2H), | |
| | | 4.15 (s, 2H); LCMS (M − H): 399.95 | |
| 86 | 3-(trifluoromethyl)-N-(4-((5- | 1H-NMR (400 MHz, DMSO-D6) δ | 87 mg, |
| | (trifluoromethyl)-1,2,4-oxadiazol-3- | 10.45 (s, 1H), 8.03 (t, 2H), 7.94 (s, | 19% yield |
| | yl)methyl)phenyl)benzenesulfonamide | 1H), 7.83 (d, 1H), 7.25-7.22 (m, | |
| | | 2H), 7.07-7.05 (m, 2H), 4.17 (s, | |
| | | 2H); LCMS (M − H): 450.00 | |

### Example 5:—Preparation of N-(4-fluorobenzyl)-4-((5-(trifluoromethyl)-1,2,4-oxadiazol-3-yl)methyl)benzamide. (Compound No. 12)

### Step 1:—Preparation of methyl-4-(2-amino-2-(hydroxyimino)ethyl)benzoate

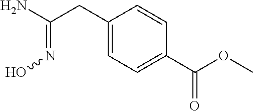

To a solution of methyl 4-(cyanomethyl)benzoate (9.5 g, 54.2 mmol) in ethanol (100 mL), hydroxylamine hydrochloride (6.8 g, 98 mmol) and sodium bicarbonate (8.2 g, 98 mmol) were added. The resulting reaction mixture was stirred at 65° C. for 18 h. The reaction mixture was filtered and the filtrate was concentrated under reduced pressure to obtain methyl-4-(2-amino-2-(hydroxyimino)ethyl)benzoate (11.2 g, 54 mmol, 99% yield).

### Step 2:—Preparation of methyl 4-((5-(trifluoromethyl)-1,2,4-oxadiazol-3-yl)methyl)benzoate

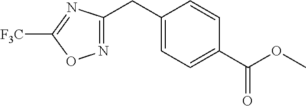 [SMILES: COC(=O)c1ccc(Cc2noc(C(F)(F)F)n2)cc1]

To a solution of methyl-4-(2-amino-2-(hydroxyimino)ethyl)benzoate (11 g, 54 mmol) in tetrahydrofuran (100 mL), trifluoroacetic anhydride (11 mL, 81 mmol) was added at 0-5° C. and stirred at 25° C. for 16 h. The resulting reaction mixture was poured into ice cold mixture of ethyl acetate (300 mL) and saturated sodium bicarbonate solution (200 mL) with stirring (caution—pH must remain basic). The ethyl acetate layer was separated, washed twice with saturated sodium bicarbonate solution (50 mL), dried over anhydrous sodium sulphate and evaporated under reduced pressure. The obtained crude product was purified on column chromatography to obtain pure methyl 4-((5-(trifluoromethyl)-1,2,4-oxadiazol-3-yl)methyl)benzoate (15 g, 31 mmol, 58% yield).

### Step 3:—Preparation of N-(4-fluorobenzyl)-4-((5-(trifluoromethyl)-1,2,4-oxadiazol-3-yl)methyl)benzamide (Compound No. 12)

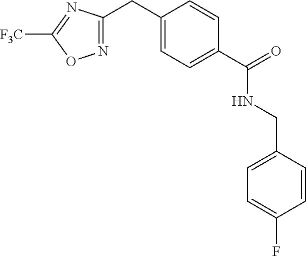 [SMILES: O=C(NCc1ccc(F)cc1)c1ccc(Cc2noc(C(F)(F)F)n2)cc1]

To a stirred solution of methyl 4-((5-(trifluoromethyl)-1,2,4-oxadiazol-3-yl)methyl)benzoate (0.25 g, 0.87 mmol) and 4-fluorobenzylamine (0.2 mL, 1.75 mmol) in toluene (7 mL), trimethylaluminum, 25% in hexane (0.58 mL, 2.2 mmol) was added at 0-5° C. under nitrogen atmosphere and stirred at 65° C. for 16 h. The reaction mixture was cooled to 25° C. and poured into the mixture of 5% aqueous acetic acid (7 mL) and ethyl acetate (15 mL) at 10° C. The mixture was then stirred at 25° C. for 10 min. The layers were separated and the aqueous layer was again extracted with ethyl acetate (20 mL). The ethyl acetate layer was collected and washed with water (20 mL), dried over anhydrous sodium sulphate and evaporated under reduced pressure. The obtain crude compound was purified on column chromatography to obtain pure N-(4-fluorobenzyl)-4-((5-(trifluoromethyl)-1,2,4-oxadiazol-3-yl)methyl)benzamide (210 mg, 0.55 mmol, 63% yield).

1H-NMR (400 MHz, DMSO-D6) δ 9.04 (t, 1H), 7.84-7.86 (m, 2H), 7.43 (d, 2H), 7.32-7.35 (m, 2H), 7.10-7.16 (m, 2H), 4.44 (d, 2H), 4.34 (s, 2H); (M+1): 380.05

TABLE 5
The following compounds were prepared by the procedure
analogous to that for the compound No. 12

| Compound | | | |
|---|---|---|---|
| No. | IUPAC Name | NMR and LCMS data | Yield |
| 13 | morpholino(4-((5- | ¹H-NMR (400 MHz, DMSO-D6) δ 7.42 | 0.17 g, |
| | (trifluoromethyl)-1,2,4- | (d, 2H), 7.38 (d, 2H), 4.32 (s, 2H), 3.59 | 57% yield |
| | oxadiazol-3- | (t, 4H), 3.47 (s, 4H); (M + 1): 342.00 | |
| | yl)methyl)phenyl)methanone | | |
| 14 | N-(3-fluorobenzyl)-4-((5- | ¹H-NMR (400 MHz, DMSO-D6) δ 9.07 | 0.20 g, |
| | (trifluoromethyl)-1,2,4- | (t, 1H), 7.86-7.88 (m, 2H), 7.44 (d, 2H), | 60% yield |
| | oxadiazol-3- | 7.33-7.38 (m, 1H), 7.14 (d, 1H), 7.03- | |
| | yl)methyl)benzamide | 7.12 (m, 2H), 4.47 (d, 2H), 4.34 (s, 2H); | |
| | | (M + 1): 380.00 | |
| 15 | N-(1-(p-tolyl)ethyl)-4-((5- | ¹H-NMR (400 MHz, DMSO-D6) δ 8.73 | 0.17 g, |
| | (trifluoromethyl)-1,2,4- | (d, 1H), 7.83-7.85 (m, 2H), 7.42 (d, 2H), | 51% yield |
| | oxadiazol-3- | 7.25 (d, 2H), 7.10 (d, 2H), 5.07-5.14 (m, | |
| | yl)methyl)benzamide | 1H), 4.33 (s, 2H), 2.25 (s, 3H), 1.43 (d, | |
| | | 3H); (M + 1): 390.05 | |
| 16 | N-(pyridin-3-ylmethyl)-4-((5- | ¹H-NMR (400 MHz, DMSO-D6) δ 9.08 | 0.25 g, |
| | (trifluoromethyl)-1,2,4- | (t, 1H), 8.53 (d, 1H), 8.44 (dd, 1H), | 48% yield |
| | oxadiazol-3- | 7.84-7.86 (m, 2H), 7.70 (dt, 1H), 7.43 | |
| | yl)methyl)benzamide | (d, 2H), 7.34 (ddd, 1H), 4.48 (d, 2H), | |
| | | 4.34 (s, 2H); (M + 1): 363.00 | |
| 17 | N-(5-chloropyridin-3-yl)-4-((5- | ¹H-NMR (400 MHz, DMSO-D6) δ 9.96 | 0.30 g, |
| | (trifluoromethyl)-1,2,4- | (s, 1H), 7.94-7.96 (m, 2H), 7.50 (d, 2H), | 89% yield |
| | oxadiazol-3- | 7.43 (d, 1H), 7.22 (d, 1H), 6.88 (dd, | |
| | yl)methyl)benzamide | 1H), 4.38 (s, 2H), 3.76 (s, 3H); (M + 1): | |
| | | 382.95 | |
| 18 | N-(2-chloro-5- | ¹H-NMR (400 MHz, DMSO-D6) δ 9.97 | 0.20 g, |
| | methoxyphenyl)-4-((5- | (s, 1H), 7.94-7.96 (m, 2H), 7.50 (d, 2H), | 54% yield |
| | (trifluoromethyl)-1,2,4- | 7.43 (d, 1H), 7.22 (d, 1H), 6.88 (dd, | |
| | oxadiazol-3- | 1H), 4.38 (s, 2H), 3.76 (s, 3H); (M + 1): | |
| | yl)methyl)benzamide | 411.95 | |
| 19 | N-(2-methoxyphenyl)-4-((5- | ¹H-NMR (400 MHz, DMSO-D6) δ 9.41 | 0.14 g, |
| | (trifluoromethyl)-1,2,4- | (s, 1H), 7.92-7.94 (m, 2H), 7.75 (dd, | 42% yield |
| | oxadiazol-3- | 1H), 7.48 (d, 2H), 7.15-7.19 (m, 1H), | |
| | yl)methyl)benzamide | 7.08 (dd, 1H), 6.96 (td, 1H), 4.37 (s, | |
| | | 2H), 3.82 (s, 3H); (M + 1): 378.20 | |
| 20 | N-(4-methoxyphenyl)-4-((5- | ¹H-NMR (400 MHz, DMSO-D6) δ | 0.20 g, |
| | (trifluoromethyl)-1,2,4- | 10.10 (s, 1H), 7.91 (d, 2H), 7.63-7.67 | 61% yield |
| | oxadiazol-3- | (m, 2H), 7.48 (d, 2H), 6.89-6.93 (m, | |
| | yl)methyl)benzamide | 2H), 4.37 (s, 2H), 3.73 (s, 3H); (M + 1): | |
| | | 378.05 | |
| 21 | N-(2-morpholinoethyl)-4-((5- | ¹H-NMR (400 MHz, DMSO-D6) δ 8.38 | 0.18 g, |
| | (trifluoromethyl)-1,2,4- | (t, 1H), 7.79 (d, 2H), 7.41 (d, 2H), 4.33 | 55% yield |
| | oxadiazol-3- | (s, 2H), 3.55 (t, 4H), 3.36 (q, 1H), 2.44 | |
| | yl)methyl)benzamide | (t, 1H), 2.39 (s, 3H); (M + 1): 385.05 | |
| 22 | N-(4-chlorophenyl)-4-((5- | ¹H-NMR (400 MHz, DMSO-D6) δ | 0.11 g, |
| | (trifluoromethyl)-1,2,4- | 10.37 (s, 1H), 7.91-7.94 (m, 2H), 7.79- | 32% yield |
| | oxadiazol-3- | 7.83 (m, 2H), 7.49-7.52 (m, 2H), 7.39- | |
| | yl)methyl)benzamide | 7.44 (m, 2H), 4.39 (s, 2H); (M − 1): | |
| | | 379.65 | |
| 23 | N-(3-fluorobenzyl)-N-methyl- | ¹H-NMR @80° C. (400 MHz, DMSO- | 0.20 g, |
| | 4-((5-(trifluoromethyl)-1,2,4- | D6) δ 7.37-7.44 (m, 5H), 7.04-7.12 (m, | 59% yield |
| | oxadiazol-3- | 3H), 4.60 (s, 2H), 4.31 (s, 2H), 3.06 (s, | |
| | yl)methyl)benzamide | 2H); (M + 1): 394.20 | |
| 24 | N-(isoxazol-3-yl)-4-((5- | ¹H-NMR (400 MHz, DMSO-D6) δ | 0.19 g, |
| | (trifluoromethyl)-1,2,4- | 11.43 (s, 1H), 8.84-8.85 (m, 1H), 7.98- | 64% yield |
| | oxadiazol-3- | 8.00 (m, 2H), 7.49 (d, 2H), 7.04 (d, 1H), | |
| | yl)methyl)benzamide | 4.38 (s, 2H); (M − 1): 336.90 | |
| 50 | N-(prop-2-yn-1-yl)-4-((5- | 1H-NMR (400 MHz, DMSO-D6) δ 8.91 | 168 mg, |
| | (trifluoromethyl)-1,2,4- | (t, 1H), 7.82 (dd, 2H), 7.43 (d, 2H), 4.34 | 52% yield |
| | oxadiazol-3- | (s, 2H), 4.04 (q, 2H), 3.11 (t, 1H); | |
| | yl)methyl)benzamide | LCMS (M + H): 309.70 | |
| 51 | N-phenyl-4-((5- | 1H-NMR (400 MHz, DMSO-D6) δ | 250 mg, |
| | (trifluoromethyl)-1,2,4- | 10.22 (s, 1H), 7.92 (dd, 2H), 7.75 (dd, | 82% yield |
| | oxadiazol-3- | 2H), 7.49 (d, 2H), 7.36-7.32 (m, 2H), | |
| | yl)methyl)benzamide | 7.11-7.07 (m, 1H), 4.38 (s, 2H); LCMS | |
| | | (M + H): 348.00 | |
| 52 | tert-butyl (4-((5- | 1H-NMR (400 MHz, DMSO-D6) δ 9.32 | 15 g, |
| | (trifluoromethyl)-1,2,4- | (s, 1H), 7.41-7.39 (m, 2H), 7.21-7.18 | 45% yield |
| | oxadiazol-3- | (m, 2H), 4.16 (s, 2H), 1.45 (s, 9H); | |
| | yl)methyl)phenyl)carbamate | LCMS (M − H): 342.00 | |
| 53 | N-(3,4-dichlorophenyl)-4-((5- | 1H-NMR (400 MHz, DMSO-D6) δ | 270 mg, |
| | (trifluoromethyl)-1,2,4- | 10.48 (s, 1H), 8.14 (d, 1H), 7.93-7.91 | 74% yield |
| | oxadiazol-3- | (m, 2H), 7.74 (dd, 1H), 7.61 (d, 1H), | |
| | yl)methyl)benzamide | 7.51 (d, 2H), 4.38 (s, 2H); LCMS | |
| | | (M + H): 416.00 | |
| 54 | N-(p-tolyl)-4-((5- | 1H-NMR (400 MHz, DMSO-D6) δ | 200 mg, |
| | (trifluoromethyl)-1,2,4- | 10.13 (s, 1H), 7.96-7.90 (m, 2H), 7.64- | 63% yield |
| | oxadiazol-3- | 7.62 (m, 2H), 7.50-7.47 (m, 2H), 7.15- | |
| | yl)methyl)benzamide | 7.13 (m, 2H), 4.37 (s, 2H), 2.26 (s, 3H); | |
| | | LCMS (M + H): 362.15 | |
| 55 | N-(3-chlorophenyl)-4-((5- | 1H-NMR (400 MHz, DMSO-D6) δ | 280 mg, |
| | (trifluoromethyl)-1,2,4- | 10.38 (s, 1H), 7.95-7.91 (m, 1H), 7.93- | 84% yield |
| | oxadiazol-3- | 7.91 (m, 2H), 7.70-7.67 (m, 1H), 7.50 | |
| | yl)methyl)benzamide | (d, 2H), 7.37 (t, 1H), 7.15 (dq, 1H), 4.38 | |
| | | (s, 2H); LCMS (M + H): 381.95 | |
| 56 | N-(4-(dimethylamino)phenyl)- | 1H-NMR (400 MHz, DMSO-D6) δ 9.95 | 290 mg, |
| | 4-((5-(trifluoromethyl)-1,2,4- | (s, 1H), 7.92 (dd, 2H), 7.55 (d, 2H), 7.46 | 85% yield |
| | oxadiazol-3- | (d, 2H), 6.73-6.70 (m, 2H), 4.37 (d, 2H), | |
| | yl)methyl)benzamide | 2.86 (s, 6H); LCMS (M + H): 391.05 | |
| 57 | N-(4-(tert-butyl)phenyl)-4-((5- | 1H-NMR (400 MHz, DMSO-D6) δ | 270 mg, |
| | (trifluoromethyl)-1,2,4- | 10.15 (s, 1H), 7.92 (dd, 2H), 7.66 (dd, | 77% yield |
| | oxadiazol-3- | 2H), 7.48 (d, 2H), 7.37-7.34 (m, 2H), | |
| | yl)methyl)benzamide | 4.37 (s, 2H), 1.27 (s, 9H); LCMS (M- | |
| | | H): 402.00 | |
| 58 | N-(m-tolyl)-4-((5- | 1H-NMR (400 MHz, DMSO-D6) δ | 250 mg, |
| | (trifluoromethyl)-1,2,4- | 10.14 (s, 1H), 7.91 (d, 2H), 7.60-7.53 | 79% yield |
| | oxadiazol-3- | (m, 2H), 7.48 (d, 2H), 7.21 (t, 1H), 6.91 | |
| | yl)methyl)benzamide | (d, 1H), 4.37 (s, 2H), 2.30 (s, 3H); | |
| | | LCMS (M − H): 360.00 | |
| 59 | 4-((5-(trifluoromethyl)-1,2,4- | 1H-NMR (400 MHz, DMSO-D6) δ | 250 mg, |
| | oxadiazol-3-yl)methyl)-N-(3- | 10.53 (s, 1H), 8.24 (s, 1H), 8.03 (d, 1H), | 69% yield |
| | (trifluoromethyl)phenyl)benz- | 7.95 (dd, 2H), 7.59 (t, 1H), 7.52 (d, 2H), | |
| | amide | 7.44 (d, 1H), 4.39 (s, 2H); LCMS | |
| | | (M − H): 413.90 | |
| 60 | N-(3-fluorophenyl)-4-((5- | 1H-NMR (400 MHz, DMSO-D6) δ | 300 mg, |
| | (trifluoromethyl)-1,2,4- | 10.40 (s, 1H), 7.93-7.90 (m, 2H), 7.74 | 94% yield |
| | oxadiazol-3- | (dt, 1H), 7.56-7.34 (m, 4H), 6.94-6.89 | |
| | yl)methyl)benzamide | (m, 1H), 4.38 (s, 2H); LCMS (M − H): | |
| | | 363.95 | |
| 61 | N-(2-fluorophenyl)-4-((5- | 1H-NMR (400 MHz, DMSO-D6) δ | 110 mg, |
| | (trifluoromethyl)-1,2,4- | 10.10 (s, 1H), 7.95 (d, 2H), 7.60-7.56 | 34% yield |
| | oxadiazol-3- | (m, 1H), 7.49 (d, 2H), 7.31-7.19 (m, | |
| | yl)methyl)benzamide | 3H), 4.38 (s, 2H); LCMH (M − H): 364.00 | |
| 62 | N-(4-fluorophenyl)-4-((5- | 1H-NMR (400 MHz, DMSO-D6) δ | 270 mg, |
| | (trifluoromethyl)-1,2,4- | 10.27 (s, 1H), 7.91 (dd, 2H), 7.80-7.74 | 85% yield |
| | oxadiazol-3- | (m, 2H), 7.49 (d, 2H), 7.21-7.15 (m, | |
| | yl)methyl)benzamide | 2H), 4.37 (s, 2H); LCMS (M − H): 363.75 | |
| 63 | N-(2,4-dichlorophenyl)-4-((5- | 1H-NMR (400 MHz, DMSO-D6) δ | 260 mg, |
| | (trifluoromethyl)-1,2,4- | 10.10 (s, 1H), 7.95 (d, 2H), 7.72 (d, 1H), | 71% yield |
| | oxadiazol-3- | 7.61 (d, 1H), 7.71-7.45 (m, 3H), 4.38 (s, | |
| | yl)methyl)benzamide | 2H); LCMS (M − H): 413.90 | |

### Example 6: Preparation of N-(m-tolyl)-4-((5-(trifluoromethyl)-1,2,4-oxadiazol-3-yl)methyl)benzothioamide (Compound No. 64)

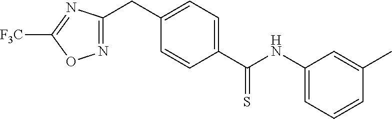 [SMILES: Cc1cccc(NC(=S)c2ccc(Cc3noc(C(F)(F)F)n3)cc2)c1]

To a solution of N-(m-tolyl)-4-((5-(trifluoromethyl)-1,2,4-oxadiazol-3-yl)methyl)benzamide (0.16 g, 0.4 mmol) in 1,4-dioxane (5 mL), 2,4-bis(4-methoxyphenyl)-1,3,2,4-dithiadiphosphetane-2,4-disulfide (0.3 g, 0.67 mmol) was added at 25° C. and stirred at 90° C. for 16 h. After completion of the reaction, the reaction mixture was quenched with sodium bicarbonate solution (50 mL) and extracted with ethyl acetate (30 mL). The ethyl acetate layer was dried over anhydrous sodium sulphate, concentrated under reduced pressure to obtain a crude product. The crude compound was purified by flash column chromatography on silica gel by using eluent 35% ethyl acetate in hexane to obtain N-(m-tolyl)-4-((5-(trifluoromethyl)-1,2,4-oxadiazol-3-yl)methyl)benzothioamide (0.1 g, 0.25 mmol, 56% yield).

1H-NMR (400 MHz, DMSO-D6) δ 11.66 (s, 1H), 7.79 (t, 2H), 7.61-7.58 (m, 2H), 7.42 (d, J 2H), 7.30 (t, 1H), 7.08 (d, 1H), 4.35 (s, 2H), 2.32 (s, 3H); LCMS (M+H): 378.30

TABLE 7
The following compounds were prepared by the procedure
analogous to that for the compound No. 64

| Compound | | | |
|---|---|---|---|
| No. | IUPAC Name | NMR and LCMS data | Yield |
| 64 | N-(m-tolyl)-4-((5-(trifluoromethyl)- | 1H-NMR (400 MHz, DMSO-D6) | 94 mg, |
| | 1,2,4-oxadiazol-3- | δ 11.66 (s, 1H), 7.79 (t, 2H), 7.61- | 56% yield |
| | yl)methyl)benzothioamide | 7.58 (m, 2H), 7.42 (d, J 2H), 7.30 | |
| | | (t, 1H), 7.08 (d, 1H), 4.35 (s, 2H), | |
| | | 2.32 (s, 3H); LCMS (M + H): | |
| | | 378.30 | |
| 65 | N-(4-(dimethylamino)phenyl)-4-((5- | 1H-NMR (400 MHz, DMSO-D6) | 113 mg, |
| | (trifluoromethyl)-1,2,4-oxadiazol-3- | δ 11.51 (s, 1H), 7.76 (d. 2H), 7.64 | 64% yield |
| | yl)methyl)benzothioamide | (d, 2H), 7.40 (d, 2H), 6.73 (d, 2H), | |
| | | 4.34 (s, 2H), 2.91 (s, 6H); LCMS | |
| | | (M + H): 407.10 | |
| 66 | N-(3-fluorophenyl)-4-((5- | 1H-NMR (400 MHz, DMSO-D6) | 140 mg, |
| | (trifluoromethyl)-1,2,4-oxadiazol-3- | δ 11.85 (s, 1H), 7.90 (d, 1H), 7.78 | 79% yield |
| | yl)methyl)benzothioamide | (d, 2H), 7.65 (d, 1H), 7.50-7.42 | |
| | | (m, 3H), 7.13-7.09 (m, 1H), 4.36 | |
| | | (s, 2H); LCMS (M + H): 381.85 | |
| 67 | N-(4-fluorophenyl)-4-((5- | 1H-NMR (400 MHz, DMSO-D6) | 135 mg, |
| | (trifluoromethyl)-1,2,4-oxadiazol-3- | δ 11.74 (s, 1H), 7.83-7.79 (m, 4H), | 65% yield |
| | yl)methyl)benzothioamide | 7.43 (d, 2H), 7.29-7.23 (m, 2H), | |
| | | 4.35 (s, 2H); LCMS (M + H): | |
| | | 381.95 | |
| 71 | N-(3-fluorobenzyl)-4-((5- | 1H-NMR (400 MHz, DMSO-D6) | 120 mg |
| | (trifluoromethyl)-1,2,4-oxadiazol-3- | δ 10.76 (t, 1H), 7.77 (dt, 2H), 7.35- | 38% yield |
| | yl)methyl)benzothioamide | 7.43 (m, 3H), 7.07-7.20 (m, 3H), | |
| | | 4.97 (d, 2H), 4.33 (s, 2H); LCMS | |
| | | (M + H): 396.25 | |

### Example 7:—Preparation of 4-methoxy-N-(4-(5-(trifluoromethyl)-1,2,4-oxadiazole-3-carbonyl)phenyl)benzamide (Compound No. 25)

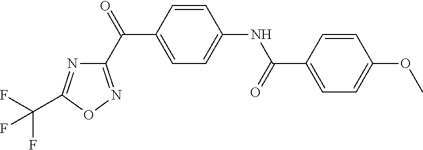 [SMILES: COc1ccc(C(=O)Nc2ccc(C(=O)c3noc(C(F)(F)F)n3)cc2)cc1]

### Step 1:—Preparation of tert-butyl (4-(5-(trifluoromethyl)-1,2,4-oxadiazole-3-carbonyl)phenyl)carbamate

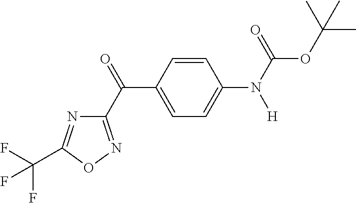 [SMILES: CC(C)(C)OC(=O)Nc1ccc(C(=O)c2noc(C(F)(F)F)n2)cc1]

The stirred solution of tert-butyl (4-((5-(trifluoromethyl)-1,2,4-oxadiazol-3-yl)methyl)phenyl)carbamate (0.2 g, 0.58 mmol), tert-butylhydroperoxide (70% in water) (0.48 ml, 3.5 mmol) and copper (II) acetate monohydrate (5.8 mg, 0.03 mmol) in tert-butanol (2 ml) was heated at 50° C. for 30 h. After completion of the reaction, the reaction mixture was quenched with water (6 mL) and extracted with dichloromethane (15 mL). The dichloromethane layer was washed with water (10 mL), brine solution (10 mL), dried over anhydrous sodium sulphate and concentrated under reduced pressure to obtain a crude compound. To the obtained crude compound, dichloromethane (5 mL) and triethylamine (0.974 ml, 6.99 mmol) were added and stirred for 5 h at 25° C. After completion of the reaction, the reaction mixture was quenched with water (15 mL), extracted with dichloromethane (30 mL). The dichloromethane layer was washed with water (10 mL), brine solution (10 mL), dried over anhydrous sodium sulphate and concentrated under reduced pressure. The crude product was purified by column chromatography on silica gel to obtain tert-butyl (4-(5-(trifluoromethyl)-1,2,4-oxadiazole-3-carbonyl)phenyl)carbamate (81% yield, 168 mg).

### Step 2: (4-aminophenyl)(5-(trifluoromethyl)-1,2,4-oxadiazol-3-yl)methanone

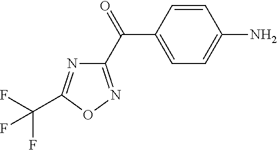 [SMILES: Nc1ccc(C(=O)c2noc(C(F)(F)F)n2)cc1]

To a solution of tert-butyl (4-(5-(trifluoromethyl)-1,2,4-oxadiazole-3-carbonyl)phenyl)carbamate (3.5 g, 9.8 mmol) in dichloromethane (20 ml), trifluoroacetic acid (6 ml, 78 mmol) was added at 0-5° C. and stirred at 25° C. for 3 h. The reaction mixture was concentrated under reduced pressure at 50° C. then diluted with dichloromethane (100 mL). The obtained solution was poured over aqueous saturated sodium bicarbonate solution (100 mL). The dichloromethane layer was washed with water (50 mL), brine solution (50 mL), dried over anhydrous sodium sulpahte, and evaporated under reduced pressure to obtain (4-aminophenyl)(5-(trifluoromethyl)-1,2,4-oxadiazol-3-yl)methanone (2.4 g, 95% yield).

### Step 3: 4-methoxy-N-(4-(5-(trifluoromethyl)-1,2,4-oxadiazole-3-carbonyl)phenyl)benzamide (Compound No. 25)

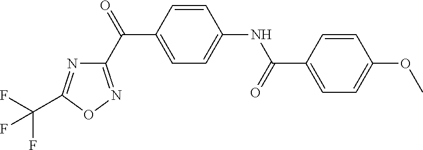 [SMILES: COc1ccc(C(=O)Nc2ccc(C(=O)c3noc(C(F)(F)F)n3)cc2)cc1]

To a solution of 4-anisic acid (142 mg, 0.9 mmol) in dichloromethane (2.5 mL), 1-(3-dimethylaminopropyl)-3-ethylcarbodiimide hydrochloride (298 mg, 1.5 mmol), 4-dimethylaminopyridine (285 mg, 2.3 mmol) were added. After 20 min of stirring, (4-aminophenyl)(5-(trifluoromethyl)-1,2,4-oxadiazol-3-yl)methanone (200 mg, 0.8 mmol) was added and the resulting reaction mixture was stirred at 25° C. for 16 h. After completion of the reaction, the reaction mixture was quenched with water. The aqueous layer was extracted thrice with dichloromethane (25 mL). The combined dichloromethane layer was washed with water (25 mL), brine solution (25 mL), dried over anhydrous sodium sulphate and evaporated under reduced pressure to obtain the residue. The residue was then purified by column chromatography using 0-50% ethyl acetate/hexane as an eluent to give 4-methoxy-N-(4-(5-(trifluoromethyl)-1,2,4-oxadiazole-3-carbonyl)phenyl)benzamide (0.16 g, 0.4 mmol, 54% yield).

TABLE 8
The following compounds were prepared by the procedure
analogous to that for the Compound No. 25

| Compound | | | |
|---|---|---|---|
| No. | IUPAC Name | NMR and LCMS data | Yield |
| 25 | 4-methoxy-N-(4-(5- | 1H-NMR (400 MHz, DMSO- | 0.17 g, |
| | (trifluoromethyl)-1,2,4- | D6) δ 10.59 (s, 1H), 8.19 (dd, | 55% yield |
| | oxadiazole-3- | 2H), 8.07-8.05 (m, 2H), 7.99 | |
| | carbonyl)phenyl)benzamide | (dd, 2H), 7.10-7.08 (m, 2H), | |
| | | 3.85 (s, 3H); LCMS (M + H): | |
| | | 391.9 | |
| 26 | 4-chloro-N-(4-(5-(trifluoromethyl)- | 1H-NMR (400 MHz, DMSO- | 0.17 g, |
| | 1,2,4-oxadiazole-3- | D6) δ 10.80 (s, 1H), 8.21 (d, | 51% yield |
| | carbonyl)phenyl)benzamide | 2H), 8.06-8.00 (m, 4H), 7.63- | |
| | | 7.66 (m, 2H); LCMS (M + H): | |
| | | 395.6 | |
| 27 | N-(4-(5-(trifluoromethyl)-1,2,4- | 1H-NMR (400 MHz, DMSO- | 0.17 g, |
| | oxadiazole-3- | D6) δ 10.98 (s, 1H), 8.82 (q, | 60% yield |
| | carbonyl)phenyl)isonicotinamide | 2H), 8.23 (dt, 2H), 8.07-8.05 | |
| | | (m, 2H), 7.88 (q, 2H); LCMS | |
| | | (M + H): 362.6 | |
| 28 | N-(4-(5-(trifluoromethyl)-1,2,4- | 1H-NMR (400 MHz, DMSO- | 0.2 g, |
| | oxadiazole-3- | D6) δ 10.94 (s, 1H), 9.15 (d, | 56.2% yield |
| | carbonyl)phenyl)nicotinamide | 1H), 8.81 (dd, 1H), 8.36-8.33 | |
| | | (m, 1H), 8.25-8.23 (m, 2H), | |
| | | 8.07 (dd, 2H), 7.62 (ddd, 1H); | |
| | | LCMS (M + H): 362.2 | |
| 29 | tert-butyl (4-(5-(trifluoromethyl)- | 1H-NMR (400 MHz, DMSO- | 213 mg, |
| | 1,2,4-oxadiazole-3- | D6) δ 10.05 (s, 1H), 8.13-8.10 | 79% yield |
| | carbonyl)phenyl)carbamate | (m, 2H), 7.71 (dd, 2H), 1.51 | |
| | | (s, 9H); LCMS (M + H): 358.2 | |
| 30 | tert-butyl (4-(difluoro(5- | 1H-NMR (400 MHz, DMSO- | 195 mg |
| | (trifluoromethyl)-1,2,4-oxadiazol-3- | D6) δ 9.73 (s, 1H), 7.64 (d, | 51% yield |
| | yl)methyl)phenyl)carbamate | 2H), 7.56 (d, 2H), 1.65- | |
| | | 1.49(m, 9H); GCMS (M): | |
| | | 379.0 | |

### Example 8: Preparation of N-(4-(difluoro(5-(trifluoromethyl)-1,2,4-oxadiazol-3-yl)methyl)phenyl)-4-(trifluoromethyl)benzamide (Compound No. 32)

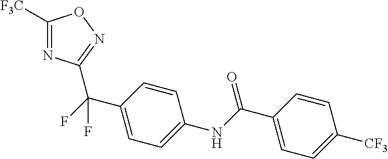 [SMILES: O=C(Nc1ccc(C(F)(F)c2noc(C(F)(F)F)n2)cc1)c1ccc(C(F)(F)F)cc1]

### Step-1: Preparation of tert-butyl (4-(difluoro(5-(trifluoromethyl)-1,2,4-oxadiazol-3-yl)methyl)phenyl)carbamate

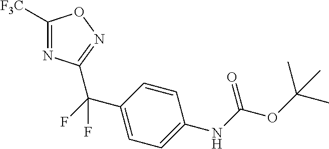 [SMILES: CC(C)(C)OC(=O)Nc1ccc(C(F)(F)c2noc(C(F)(F)F)n2)cc1]

To the stirred solution of tert-butyl (4-(5-(trifluoromethyl)-1,2,4-oxadiazole-3-carbonyl)phenyl)carbamate (6.5 g, 18.2 mmol) in dichloromethane (65 mL), diethylamino sulfur trifluoride (7.2 mL, 54.6 mmol) was added at 0-5° C. under nitrogen atmosphere and stirred for 24 h at 25° C. After completion of the reaction, the reaction mixture was quenched by aqueous saturated sodium carbonate solution (100 mL). The sodium carbonate layer was extracted thrice with dichloromethane (75 mL), washed with water (25 mL), brine solution (25 mL), dried over anhydrous sodium sulphate and evaporated under reduced pressure to obtain a crude compound which was purified by flash column chromatography to obtain tert-butyl (4-(difluoro(5-(trifluoromethyl)-1,2,4-oxadiazol-3-yl)methyl)phenyl)carbamate (5 g, 72% yield, 13.2 mmol).

### Step-2: 4-(difluoro(5-(trifluoromethyl)-1,2,4-oxadiazol-3-yl)methyl)aniline hydrochloride

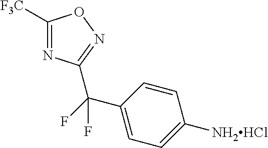

To a stirred solution of tert-butyl (4-(difluoro(5-(trifluoromethyl)-1,2,4-oxadiazol-3-yl)methyl)phenyl)carbamate (2 g, 5.3 mmol) in dichloromethane (20 mL), hydrochloric acid solution in 1,4-dioxane (4M solution in dioxane, 5 mL) was added at 0-5° C. and the reaction mixture was stirred at 25° C. for 3 h under nitrogen atmosphere. The reaction mixture was concentrated under reduced pressure. The crude product was stirred in n-hexane (30 mL) at 25° C. for 20 min, filtered and dried under reduced pressure to obtain 4-(difluoro(5-(trifluoromethyl)-1,2,4-oxadiazol-3-yl)methyl)aniline hydrochloride (1.2 g, 82% yield, 4.3 mmol).

### Step-3: Preparation of N-(4-(difluoro(5-(trifluoromethyl)-1,2,4-oxadiazol-3-yl)methyl)phenyl)-4-(trifluoromethyl)benzamide

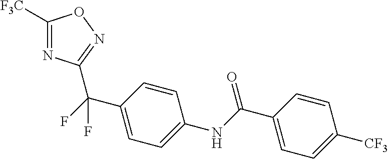 [SMILES: O=C(Nc1ccc(C(F)(F)c2noc(C(F)(F)F)n2)cc1)c1ccc(C(F)(F)F)cc1]

To the stirred solution of 4-(difluoro(5-(trifluoromethyl)-1,2,4-oxadiazol-3-yl)methyl) aniline hydrochloride (150 mg, 0.48 mmol) in dichloromethane (10 mL), 4-(trifluoromethyl)benzoyl chloride (0.1 mL, 0.5 mmol) was added at 0-5° C. and stirred at 25° C. for 3 h under nitrogen atmosphere. The reaction mixture was quenched with water (10 mL). The dichloromethane layer was separated, was washed with water (10 mL), brine solution (10 mL), dried over anhydrous sodium sulphate and concentrated under reduced pressure. The crude product obtained was purified by column chromatography to obtain N-(4-(difluoro(5-(trifluoromethyl)-1,2,4-oxadiazol-3-yl)methyl)phenyl)-4-(trifluoromethyl)benzamide (114 mg, 53% yield).

1H-NMR (400 MHz, DMSO-D6) δ 10.77 (s, 1H), 8.17 (d, 2H), 8.00 (d, 22H), 7.95 (d, 2H), 7.71 (d, 2H); LCMS (M−H): 449.95

TABLE 9
The following compounds were prepared by the procedure
analogous to that for the Compound No. 32

| Compound | | | |
|---|---|---|---|
| No. | IUPAC Name | NMR and LCMS data | Yield |
| 33 | N-(4-(difluoro(5-(trifluoromethyl)- | 1H-NMR (400 MHz, DMSO- | 105 mg, |
| | 1,2,4-oxadiazol-3-yl)methyl)phenyl)-2- | D6) δ 10.48 (s, 1H), 7.78 (d, | 42% yield |
| | phenylacetamide | 2H), 7.60 (d, 2H), 7.32-7.22 | |
| | | (m, 5H), 3.67 (s, 2H), ; LCMS | |
| | | (M − H): 395.95 | |
| 34 | N-(4-(difluoro(5-(trifluoromethyl)- | 1H-NMR (400 MHz, DMSO- | 158 mg, |
| | 1,2,4-oxadiazol-3-yl)methyl)phenyl)-4- | D6) δ 10.57 (s, 1H), 8.08-7.98 | 62% yield |
| | fluorobenzamide | (m, 4H), 7.69 (d, 2H), 7.42- | |
| | | 7.38 (m, 2H); LCMS (M − H): | |
| | | 399.70 | |
| 35 | N-(4-(difluoro(5-(trifluoromethyl)- | 1H-NMR (400 MHz, DMSO- | 178 mg, |
| | 1,2,4-oxadiazol-3- | D6) δ 10.56 (s, 1H), 8.02-7.96 | 59% yield |
| | yl)methyl)phenyl)benzamide | (m, 4H), 7.70-7.54 (m, 5H); | |
| | | LCMS (M − H): 382.00 | |
| 36 | N-(4-(difluoro(5-(trifluoromethyl)- | 1H-NMR (400 MHz, DMSO- | 180 mg, |
| | 1,2,4-oxadiazol-3-yl)methyl)phenyl)-2- | D6) δ 10.49 (s, 1H), 7.79 (d, | 46% yield |
| | (4-fluorophenyl) acetamide | 2H), 7.62 (d, 2H), 7.38-7.35 | |
| | | (m, 2H), 7.18-7.14 (m, 2H), | |
| | | 3.69 (s, 2H); LCMS (M − H): | |
| | | 413.90 | |
| 37 | 4-cyano-N-(4-(difluoro(5- | 1H-NMR (400 MHz, DMSO- | 180 mg, |
| | (trifluoromethyl)-1,2,4-oxadiazol-3- | D6) δ 10.78 (s, 1H), 8.13 (dd, | 46% yield |
| | yl)methyl)phenyl)benzamide | 2H), 8.06 (dd, 2H), 7.99 | |
| | | (d,2H), 7.71 (d, 2H); LCMS | |
| | | (M − H): 407.00 | |
| 38 | N-(4-(difluoro(5-(trifluoromethyl)- | 1H-NMR (400 MHz, DMSO- | 180 mg, |
| | 1,2,4-oxadiazol-3-yl)methyl)phenyl)-4- | D6) δ 10.46 (d, 1H), 8.06-7.99 | 95% yield |
| | methylbenzamide | (m, 2H), 7.91-7.88 (m, 2H), | |
| | | 7.69-7.65 (m, 2H), 7.37-7.34 | |
| | | (m, 2H), 2.38 (s, 3H); LCMS | |
| | | (M − H): 396.00 | |
| 39 | N-(4-(difluoro(5-(trifluoromethyl)- | 1H-NMR (400 MHz, DMSO- | 125 mg, |
| | 1,2,4-oxadiazol-3- | D6) δ 10.98 (s, 1H), 8.78 (dq, | 34% yield |
| | yl)methyl)phenyl)picolinamide | 1H), 8.20-8.08 (m, 4H), 7.73- | |
| | | 7.68 (m, 3H), ; LCMS (M − H): | |
| | | 383.00 | |

### Example 9: Preparation of N-(4-((5-(trifluoromethyl)-1,2,4-oxadiazol-3-yl)methyl)benzyl)cyclopropanecarboxamide. (Compound No. 93)

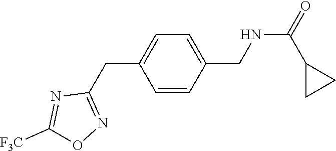 [SMILES: O=C(NCc1ccc(Cc2noc(C(F)(F)F)n2)cc1)C1CC1]

### Step 1:—Preparation of N′-hydroxy-2-(p-tolyl)acetimidamide

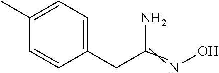

To a stirred solution of 2-(p-tolyl)acetonitrile (10.08 mL, 76 mmol) in ethanol (85 mL) was added sodium bicarbonate (11.53 g, 137 mmol) and hydroxylamine hydrochloride (9.54 g, 137 mmol) at 0° C. under nitrogen atmosphere. The resulting reaction mixture was allowed to stir at 70° C. for 16 h. After completion of the reaction, ethyl acetate (10 mL) was added and filtered through sintered glass funnel. The filtrate was evaporated under reduced pressure to obtain N′-hydroxy-2-(p-tolyl)acetimidamide (12.45 g, 76 mmol, 99% yield).

### Step 2:—Preparation of 3-(4-methylbenzyl)-5-(trifluoromethyl)-1,2,4-oxadiazole

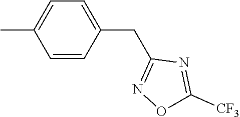 [SMILES: Cc1ccc(Cc2noc(C(F)(F)F)n2)cc1]

To a stirred solution of N′-hydroxy-2-(p-tolyl)acetimidamide (12.5 g, 76 mmol) in tetrahydrofuran (100 mL) was added trifluoroacetic anhydride (15.05 mL, 107 mmol) slowly at 0° C. under nitrogen atmosphere. The reaction mixture was stirred at 25° C. for 16 h. After completion of the reaction, the reaction mixture was poured into a beaker containing sodium bicarbonate (19.18 g, 228 mmol) dissolved in ice water (300 mL) along with ethyl acetate (150 mL). The ethyl acetate layer was separated, dried over anhydrous sodium sulphate and evaporated under reduced pressure to get a crude residue. The crude residue was purified using column chromatography to obtain pure 3-(4-methylbenzyl)-5-(trifluoromethyl)-1,2,4-oxadiazole (6.3 g, 26.0 mmol, 34% yield).

### Step 3:—Preparation of 3-(4-(bromomethyl)benzyl)-5-(trifluoromethyl)-1,2,4-oxadiazole

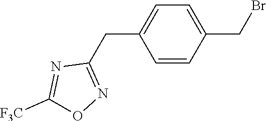 [SMILES: FC(F)(F)c1nc(Cc2ccc(CBr)cc2)no1]

The N-bromosuccinimide (6.92 g, 38.9 mmol) was slowly added to solution of 3-(4-methylbenzyl)-5-(trifluoromethyl)-1,2,4-oxadiazole (6.28 g, 25.9 mmol) in chloroform (60 mL) at 25° C. To this mixture, azobisisobutyronitrile (2.98 g, 18.15 mmol) was added and resulting solution were stirred at 50° C. for 16 h. Upon completion, the reaction was diluted with dichloromethane (20 mL) and treated twice with saturated sodium bicarbonate (20 mL) solution. The organic layer was separated, dried over anhydrous sodium sulphate and evaporated under vacuum to get crude product which upon purification obtained 3-(4-(bromomethyl)benzyl)-5-(trifluoromethyl)-1,2,4-oxadiazole (6.81 g, 21.21 mmol, 82% yield).

### Step 4:—Preparation of 3-(4-(azidomethyl)benzyl)-5-(trifluoromethyl)-1,2,4-oxadiazole

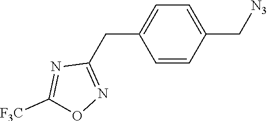 [SMILES: [N-]=[N+]=NCc1ccc(Cc2noc(C(F)(F)F)n2)cc1]

The sodium azide (1.594 g, 24.53 mmol) was slowly added to solution of 3-(4-(bromomethyl)benzyl)-5-(trifluoromethyl)-1,2,4-oxadiazole (6.3 g, 19.62 mmol) in N,N-dimethylformamide (45 mL) at 25° C. The resulting solution were stirred at 40° C. for 16 h. Upon completion, the reaction was quenched with crushed ice and extracted thrice with ethyl acetate (150 mL). The ethyl acetate layer was separated, dried over anhydrous sodium sulphate. The organic layer was evaporated under reduced pressure to get the compound 3-(4-(azidomethyl)benzyl)-5-(trifluoromethyl)-1,2,4-oxadiazole (5.4 g, 19.1 mmol, 97% yield)

### Step 5:—Preparation of (4-((5-(trifluoromethyl)-1,2,4-oxadiazol-3-yl)methyl)phenyl)methanamine

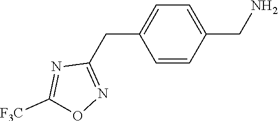 [SMILES: NCc1ccc(Cc2noc(C(F)(F)F)n2)cc1]

Triphenylphosphine (7.36 g, 28.1 mmol) was slowly added to solution of 3-(4-(azidomethyl)benzyl)-5-(trifluoromethyl)-1,2,4-oxadiazole (5.3 g, 18.71 mmol) in tetrahydrofuran (50 mL) at 0° C. Water was added and resulting solution was stirred at 70° C. for 16 h. Upon completion, the reaction mixture was concentrated under vacuum to get crude product and direct column purification of the crude compound yielded (4-((5-(trifluoromethyl)-1,2,4-oxadiazol-3-yl)methyl)phenyl)methanamine (2.6 g, 10.1 mmol, 54% yield).

### Step 6:—Preparation of N-(4-((5-(trifluoromethyl)-1,2,4-oxadiazol-3-yl)methyl)benzyl)cyclopropanecarboxamide. (Compound No. 93)

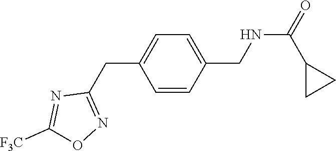 [SMILES: O=C(NCc1ccc(Cc2noc(C(F)(F)F)n2)cc1)C1CC1]

To the solution of (4-((5-(trifluoromethyl)-1,2,4-oxadiazol-3-yl)methyl)phenyl)methanamine (0.2 g, 0.8 mmol) in dichloromethane (6 mL), triethylamine (0.3 mL, 2 mmol) was added at 25° C. After 10 min of stirring cyclopropanecarboxylic acid chloride (0.1 mL, 1 mmol) was added at 25° C. and the resulting reaction mixture was stirred for 3 h. After completion of the reaction, the reaction mixture was diluted with dichloromethane (30 mL), washed twice with saturated sodium bicarbonate (10 mL) solution, dried over anhydrous sodium sulphate and evaporated under reduced pressure to obtain a crude product. The crude product obtained was purified by using column chromatography to obtain N-(4-((5-(trifluoromethyl)-1,2,4-oxadiazol-3-yl)methyl)benzyl)cyclopropanecarboxamide (0.1 g, 0.3 mmol, 43% yield).

1H-NMR (400 MHz, DMSO-D6) δ 8.52 (t, 1H), 7.28 (d, 2H), 7.21 (d, 2H), 4.24 (d, 4H), 1.61-1.54 (m, 1H), 0.69-0.61 (m, 4H); LCMS(M+H): 326.30

TABLE 10
The following compounds were prepared by the procedure
analogous to that for the Compound No. 93

| Compound | | | |
|---|---|---|---|
| No. | IUPAC Name | NMR and LCMS data | Yield |
| 94 | 4-methyl-N-(4-((5- | 1H-NMR (400 MHz, DMSO- | 135 mg, |
| | (trifluoromethyl)-1,2,4- | D6) δ 8.93 (t, 1H), 7.82-7.76 | 46% |
| | oxadiazol-3- | (m, 2H), 7.28-7.25 (m, 6H), | |
| | yl)methyl)benzyl)benzamide | 4.46-4.42 (m, 2H), 4.22 (s, 2H), | |
| | | 2.35-2.32 (m, 3H); LCMS | |
| | | (M + H): 376 | |
| 95 | 2-fluoro-N-(4-((5- | 1H-NMR (400 MHz, DMSO- | 130 mg, |
| | (trifluoromethyl)-1,2,4- | D6) δ 8.87-8.83 (m, 1H), 7.64- | 44% |
| | oxadiazol-3- | 7.60 (m, 1H), 7.55-7.49 (m, | |
| | yl)methyl)benzyl)benzamide | 1H), 7.30-7.25 (m, 6H), 4.44 (d, | |
| | | 2H), 4.24 (s, 2H); LCMS | |
| | | (M + H): 380 | |
| 96 | 3-fluoro-N-(4-((5- | 1H-NMR (400 MHz, DMSO- | 120 mg, |
| | (trifluoromethyl)-1,2,4- | D6) δ 9.12(t, 1H), 7.73 (dt, | 41% |
| | oxadiazol-3- | 1H), 7.66 (dq, 1H), 7.52 (td, | |
| | yl)methyl)benzyl)benzamide | 1H), 7.40-7.35 (m, 1H), 7.29 (s, | |
| | | 4H), 4.45 (d, 2H), 4.23 (s, 2H); | |
| | | LCMS (M + H): 380 | |
| 97 | 3-chloro-N-(4-((5- | 1H-NMR (400 MHz, DMSO- | 185 mg, |
| | (trifluoromethyl)-1,2,4- | D6) δ 9.15(t, 1H), 7.92 (t, 1H), | 60% |
| | oxadiazol-3- | 7.83 (dt, 1H), 7.60 (dq, 1H), | |
| | yl)methyl)benzyl)benzamide | 7.50 (t, 1H), 7.29 (s, 4H), 4.45 | |
| | | (d, 2H), 4.23 (s, 2H); LCMS | |
| | | (M + H): 396 | |
| 98 | N-(4-((5-(trifluoromethyl)- | 1H-NMR (400 MHz, DMSO- | 90 mg, |
| | 1,2,4-oxadiazol-3- | D6) δ 8.23 (t, 1H), 7.26 (d, 2H), | 37% |
| | yl)methyl)benzyl)propion- | 7.19 (d, 2H), 4.21 (d, 4H),2.11 | |
| | amide | (q, 2H), 1.02-0.97 (m, 3H); | |
| | | LCMS (M + H): 314 | |

### Example 10:—Preparation of N-phenyl-4-(2-(5-(trifluoromethyl)-1,2,4-oxadiazol-3-yl)propan-2-yl)benzamide (Compound No. 82)

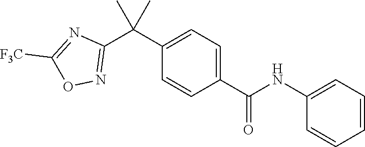 [SMILES: CC(C)(c1ccc(C(=O)Nc2ccccc2)cc1)c1noc(C(F)(F)F)n1]

### Step 1: Preparation of 4-(2-Cyanopropan-2-yl)benzoic acid

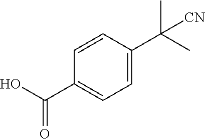

To a stirred suspension of sodium hydride (4.4 g, 111 mmol) in tetrahydrofuran (100 mL), methyl-4-(cyanomethyl)benzoate (6.5 g, 37.1 mmol) in tetrahydrofuran (30 mL) was added portion wise at 0° C. and stirred for 30 min. Iodomethane (5.8 mL, 93 mmol) was added at 0° C. and the resulting reaction mixture was stirred at 25° C. for 12 h. After completion of the reaction, the reaction mixture was quenched with ice cold water (40 mL) and extracted with ethyl acetate (100 mL). The aqueous layer was separated and acidified with 10% of hydrochloric acid and extracted twice with ethyl acetate (80 mL). The combined ethyl acetate layer was dried over anhydrous sodium sulphate and concentrated under reduced pressure to obtain 4-(2-cyanopropan-2-yl)benzoic acid (5.8 g, 31 mmol, 83% yield).

### Step 2: Preparation of 4-(1-Amino-1-(hydroxyimino)-2-methylpropan-2-yl)benzoic acid

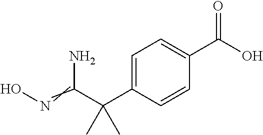 [SMILES: CC(C)(C(N)=NO)c1ccc(C(=O)O)cc1]

To a solution of 4-(2-cyanopropan-2-yl)benzoic acid (6.5 g, 34 mmol) in ethanol (50 mL), hydroxylamine (50% aqueous solution) (7.4 mL, 120 mmol) was added at 25° C. and stirred at 65° C. for 16 h. The resulting reaction mixture was concentrated under reduced pressure to obtain 4-(1-amino-1-(hydroxyimino)-2-methylpropan-2-yl)benzoic acid (7.2 g, 34 mmol, 95% yield).

### Step 3: Preparation of 4-(2-(5-(trifluoromethyl)-1,2,4-oxadiazol-3-yl)propan-2-yl)benzoic acid

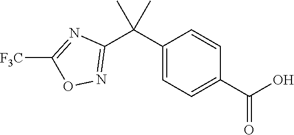 [SMILES: CC(C)(c1ccc(C(=O)O)cc1)c1noc(C(F)(F)F)n1]

To a suspension of 4-(1-amino-1-(hydroxyimino)-2-methylpropan-2-yl)benzoic acid (6.5 g, 29 mmol) in tetrahydrofuran (10 mL), trifluoroacetic anhydride (0.3 mL, 2 mmol) was added at 0° C. under nitrogen atmosphere. The resulting reaction mixture was stirred at 25° C. for 16 h. After completion of the reaction, the reaction mixture was poured into ice cold water (20 mL) and extracted twice with ethyl acetate (80 mL). The ethyl acetate layer was separated, dried over anhydrous sodium sulphate and concentrated under reduced pressure. The crude product obtained was purified by column chromatography on silica gel using hexane to 20% ethyl acetate in hexane as an eluent to obtain 4-(2-(5-(trifluoromethyl)-1,2,4-oxadiazol-3-yl)propan-2-yl)benzoic acid (5.3 g, 17.6 mmol, 60% yield).

### Step 4: Preparation of N-phenyl-4-(2-(5-(trifluoromethyl)-1,2,4-oxadiazol-3-yl)propan-2-yl)benzamide

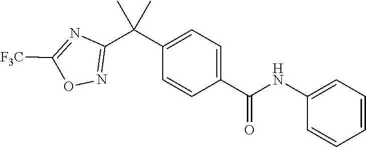 [SMILES: CC(C)(c1ccc(C(=O)Nc2ccccc2)cc1)c1noc(C(F)(F)F)n1]

To a solution of 4-(2-(5-(trifluoromethyl)-1,2,4-oxadiazol-3-yl)propan-2-yl)benzoic acid (0.3 g, 1 mmol) in dichloromethane (30 mL), 4-dimethylaminopyridine (0.3 g, 2.5 mmol), 1-(3-dimethylaminopropyl)-3-ethylcarbodiimide hydrochloride (0.4 g, 2.0 mmol) and aniline (0.12 g, 1.3 mmol) were added at 0-5° C. under nitrogen atmosphere and stirred at 25° C. for 18 h. The reaction mixture was diluted with dichloromethane (20 mL), washed twice with water (30 mL), dried over anhydrous sodium sulphate and concentrated under reduced pressure to obtain a crude product. The crude product was purified by flash column chromatography on silica gel using eluent 35% ethyl acetate in hexane to obtain N-phenyl-4-(2-(5-(trifluoromethyl)-1,2,4-oxadiazol-3-yl)propan-2-yl)benzamide (0.25 g, 0.7 mmol, 67% yield).

1H-NMR (400 MHz, CHLOROFORM-D) δ 7.85-7.82 (m, 2H), 7.77 (s, 1H), 7.63-7.60 (m, 2H), 7.47-7.44 (m, 2H), 7.40-7.43 (m, 2H), 7.18-7.13 (m, 1H), 1.86 (s, 6H); LCMS (M+H): 376.15

TABLE 11
The following compounds were prepared by the procedure
analogous to that for the Compound No. 82

| Compound | | | |
|---|---|---|---|
| No. | IUPAC Name | NMR and LCMS data | Yield |
| 82 | N-phenyl-4-(2-(5-(trifluoromethyl)- | 1H-NMR (400 MHz, | 250 mg, |
| | 1,2,4-oxadiazol-3-yl)propan-2- | CHLOROFORM-D) δ 7.85-7.82 | 67% yield |
| | yl)benzamide | (m, 2H), 7.77 (s, 1H), 7.63-7.60 | |
| | | (m, 2H), 7.47-7.44 (m, 2H), 7.40- | |
| | | 7.43 (m, 2H), 7.18-7.13 (m, 1H), | |
| | | 1.86 (s, 6H); LCMS (M + H): | |
| | | 376.15 | |
| 83 | N-(p-tolyl)-4-(2-(5-(bifluoromethyl)- | 1H-NMR (400 MHz, | 241 mg, |
| | 1,2,4-oxadiazol-3-yl)propan-2- | CHLOROFORM-D) δ 7.85-7.82 | 62% yield |
| | yl)benzamide | (m, 2H), 7.72 (s, 1H), 7.51-7.43 | |
| | | (m, 4H), 7.17 (d, 2H), 2.35 (d, | |
| | | 3H), 1.86 (s, 6H); LCMS (M + H): | |
| | | 389.95 | |
| 84 | N-(4-chlorophenyl)-4-(2-(5- | 1H-NMR (400 MHz, | 386 mg, |
| | (trifluoromethyl)-1,2,4-oxadiazol-3- | CHLOROFORM-D) δ 7.85-7.79 | 94% yield |
| | yl)propan-2-yl)benzamide | (m, 3H), 7.59-7.56 (m, 2H), 7.48- | |
| | | 7.43 (m, 2H), 7.36-7.31 (m, 2H), | |
| | | 1.86 (s, 6H); LCMS (M + H): | |
| | | 410.05 | |
| 85 | N-(pyridin-4-yl)-4-(2-(5- | 1H-NMR (400 MHz, | 330 mg, |
| | (trifluoromethyl)-1,2,4-oxadiazol-3- | CHLOROFORM-D) δ 8.54-8.52 | 88% yield |
| | yl)propan-2-yl)benzamide | (m, 2H), 8.12 (d, 1H), 7.86-7.83 | |
| | | (m, 2H), 7.62-7.60 (m, 2H), 7.48- | |
| | | 7.45 (m, 2H), 1.86 (s, 6H); LCMS | |
| | | (M + H): 377.30 | |
| 87 | N-(2-methoxyphenyl)-4-(2-(5- | 1H-NMR (400 MHz, | 286 mg, |
| | (trifluoromethyl)-1,2,4-oxadiazol-3- | CHLOROFORM-D) δ 8.51 (dd, | 71% yield |
| | yl)propan-2-yl)benzamide | 2H), 7.88-7.85 (m, 2H), 7.48-7.45 | |
| | | (m, 2H), 7.09 (td, 1H), 7.02 (td, | |
| | | 1H), 6.93-6.90 (m, 1H), 3.91 (s, | |
| | | 3H), 1.86 (s, 6H); LCMS (M + H): | |
| | | 406.10 | |
| 88 | N-(pyridin-3-yl)-4-(2-(5- | 1H-NMR (400 MHz, | 420 mg, |
| | (trifluoromethyl)-1,2,4-oxadiazol-3- | CHLOROFORM-D) δ 8.67-8.66 | 87% yield |
| | yl)propan-2-yl)benzamide | (m, 1H), 8.39-8.28 (m, 2H), 8.03 | |
| | | (s, 1H), 7.89-7.85 (m, 2H), 7.48- | |
| | | 7.45 (m, 2H), 7.34-7.31 (m, 1H), | |
| | | 1.86 (s, 6H); LCMS (M + H): | |
| | | 377.10 | |

### Example 11:—Preparation of N-phenyl-4-(1-(5-(trifluoromethyl)-1,2,4-oxadiazol-3-yl)cyclopropyl)benzamide (Compound No. 99)

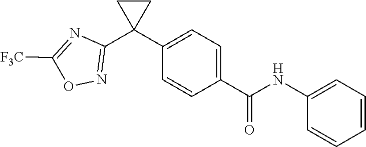 [SMILES: O=C(Nc1ccccc1)c1ccc(C2(c3noc(C(F)(F)F)n3)CC2)cc1]

### Step 1:—Preparation of 4-(1-cyanocyclopropyl)benzoic acid

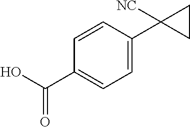 [SMILES: N#CC1(c2ccc(C(=O)O)cc2)CC1]

To a stirred suspension of sodium hydride (6.64 g, 166 mmol) in tetrahydrofuran (100 mL), methyl-4-(cyanomethyl)benzoate (8 g, 46 mmol) in tetrahydrofuran (30 mL) was added portion wise at 0° C. and stirred for 30 min. To the reaction mixture was added 1,2-dibromoethane (3.6 ml, 42 mmol) in tetrahydrofuran (10 mL) and stirred at 0° C. for 1 h and allowed to stir at 25° C. for 12 h. The reaction mixture was quenched with ice cold water slowly and diluted with ethyl acetate (100 mL). The aqueous layer was collected and acidified with 10% of hydrochloric acid and extracted twice with ethyl acetate (80 mL). The ethyl acetate layer was collected, dried over anhydrous sodium sulphate and concentrated under reduced pressure to obtain 4-(1-cyanocyclopropyl)benzoic acid (3.4 g, 18 mmol, 44% yield).

### Step 2:—Preparation of 4-(1-(N′-hydroxycarbamimidoyl)cyclopropyl)benzoic acid

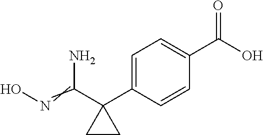 [SMILES: NC(=NO)C1(c2ccc(C(=O)O)cc2)CC1]

To a solution of 4-(1-cyanocyclopropyl)benzoic acid (3.4 g, 18 mmol) in ethanol (50 mL), hydroxylamine aqueous solution (50%) (3.9 mL, 64 mmol) was added at 25° C. and stirred at 65° C. for 16 h. The resulting reaction mixture was concentrated under reduced pressure to obtain 4-(1-(N′-hydroxycarbamimidoyl)cyclopropyl)benzoic acid (3.8 g, 17.25 mmol, 95% yield).

### Step 3:—Preparation of 4-(1-(5-(trifluoromethyl)-1,2,4-oxadiazol-3-yl)cyclopropyl)benzoic acid

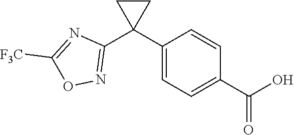 [SMILES: O=C(O)c1ccc(C2(c3noc(C(F)(F)F)n3)CC2)cc1]

To a suspension of 4-(1-(N′-hydroxycarbamimidoyl)cyclopropyl)benzoic acid (4.8 g, 21.8 mmol) in tetrahydrofuran (50 mL), trifluoroacetic anhydride (0.28 mL, 1.98 mmol) was added at 0° C. under nitrogen atmosphere. The resulting reaction mixture was stirred at 25° C. for 16 h. The reaction mixture was poured into ice cold water (50 mL) and extracted twice with ethyl acetate (80 mL). The ethyl acetate layer was dried over anhydrous sodium sulphate and concentrated under reduced pressure. The obtained crude product was purified by column chromatography on silica gel using 30% ethyl acetate in hexane as an eluent to obtain 4-(1-(5-(trifluoromethyl)-1,2,4-oxadiazol-3-yl)cyclopropyl)benzoic acid (4.5 g, 15.1 mmol, 69% yield).

### Step 4:—Preparation of N-phenyl-4-(1-(5-(trifluoromethyl)-1,2,4-oxadiazol-3-yl)cyclopropyl)benzamide

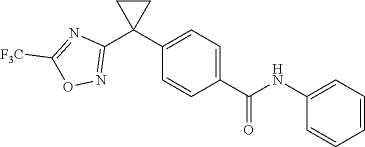 [SMILES: O=C(Nc1ccccc1)c1ccc(C2(c3noc(C(F)(F)F)n3)CC2)cc1]

To a solution of 4-(1-(5-(trifluoromethyl)-1,2,4-oxadiazol-3-yl)cyclopropyl)benzoic acid (0.2 g, 0.67 mmol) in dichloromethane (15 mL), 4-dimethylaminopyridine (0.21 g, 1.68 mmol), 1-(3-dimethylaminopropyl)-3-ethylcarbodiimide hydrochloride (0.26 g, 1.34 mmol) and aniline (0.08 g, 0.87 mmol) were added at 0-5° C. under nitrogen atmosphere and stirred at 25° C. for 18 h. The reaction mixture was diluted with dichloromethane (20 mL), washed twice with water (30 mL), dried over anhydrous sodium sulphate and concentrated under reduced pressure to obtain a crude product. The crude product was purified by flash column chromatography on silica gel using eluent 35% ethyl acetate in hexane to obtain N-phenyl-4-(1-(5-(trifluoromethyl)-1,2,4-oxadiazol-3-yl)cyclopropyl)benzamide (0.14 g, 0.36 mmol, 54% yield).

TABLE 12
The following compounds were prepared by the procedure analogous to that for the compound No. 99

| Compound | | | |
|---|---|---|---|
| no. | IUPAC Name | NMR and LCMS data | Yield |
| 99 | 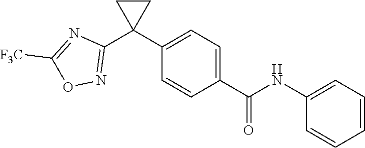 [SMILES: O=C(Nc1ccccc1)c1ccc(C2(c3noc(C(F)(F)F)n3)CC2)cc1] | N-phenyl-4-(1-(5-(trifluoromethyl)- 1,2,4-oxadiazol-3- yl)cyclopropyl)benzamide | 135 mg, 54% yield |
| 100 | 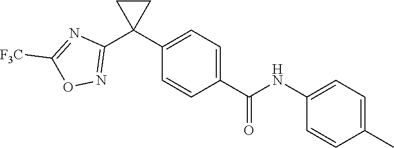 [SMILES: Cc1ccc(NC(=O)c2ccc(C3(c4noc(C(F)(F)F)n4)CC3)cc2)cc1] | N-(p-tolyl)-4-(1-(5-(trifluoromethyl)- 1,2,4-oxadiazol-3- yl)cyclopropyl)benzamide | 151 mg, 58% yield |
| 101 | 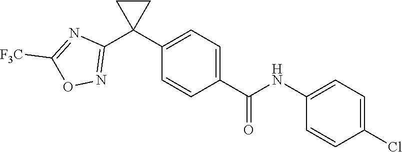 [SMILES: O=C(Nc1ccc(Cl)cc1)c1ccc(C2(c3noc(C(F)(F)F)n3)CC2)cc1] | N-(4-chlorophenyl)-4-(1-(5- (trifluoromethyl)-1,2,4-oxadiazol-3- yl)cyclopropyl)benzamide | 125 mg, 46% yield |
| 102 | 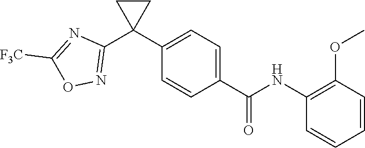 [SMILES: COc1ccccc1NC(=O)c1ccc(C2(c3noc(C(F)(F)F)n3)CC2)cc1] | N-(2-methoxyphenyl)-4-(1-(5- (trifluoromethyl)-1,2,4-oxadiazol-3- yl)cyclopropyl)benzamide | 126 mg, 47% yield |

### Example 12: Preparation of -(4-((phenylthio)methyl)benzyl)-5-(trifluoromethyl)-1,2,4-oxadiazole (Compound No. 105)

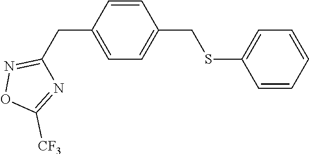 [SMILES: FC(F)(F)c1nc(Cc2ccc(CSc3ccccc3)cc2)no1]

### Step 1: Preparation of 2-(4-(bromomethyl)phenyl)acetonitrile

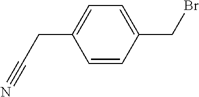 [SMILES: N#CCc1ccc(CBr)cc1]

N-bromosuccinimide (10.8 g, 61 mmol) was slowly added to the solution of 2-(p-tolyl)acetonitrile (8 g, 61 mmol) in chloroform (80 mL) at 25° C. Azobisisobutyronitrile (2.003 g, 12.20 mmol) was added and the resulting solution was stirred at 25° C. for 16 h. After completion of the reaction, the reaction mixture was diluted with dichloromethane (100 mL), washed twice with saturated aqueous sodium bicarbonate (20 mL) solution and concentrated under reduced pressure to obtain a crude product. The crude product was purified by column purification to obtain 2-(4-(bromomethyl)phenyl)acetonitrile (3 g, 14.3 mmol, 23% yield).

### Step 2: Preparation of 2-(4-((phenylthio)methyl)phenyl)acetonitrile

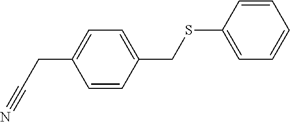 [SMILES: N#CCc1ccc(CSc2ccccc2)cc1]

Potassium tert-butoxide (0.75 g, 6.7 mmol) was slowly added to a solution of thiophenol (0.52 mL, 5 mmol) in N,N-dimethylformamide (6 mL) at 25° C. To this mixture, 2-(4-(bromomethyl)phenyl)acetonitrile (0.700 g, 3.33 mmol) was added and the resulting reaction mixture was stirred at 25° C. for 16 h. The reaction mixture was quenched by crushed ice (30 g) and extracted thrice with ethyl acetate (10 mL). The combined ethyl acetate layer was dried over sodium sulphate and concentrated under reduced pressure to obtain 2-(4-((phenylthio)methyl)phenyl)acetonitrile (0.7 g, 2.9 mmol, 88% yield).

### Step 3: Preparation of N′-hydroxy-2-(4-((phenylthio)methyl)phenyl)acetimidamide

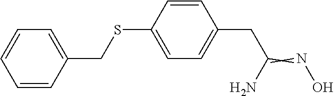 [SMILES: NC(Cc1ccc(SCc2ccccc2)cc1)=NO]

To a solution of 2-(4-((phenylthio)methyl)phenyl)acetonitrile (0.96 g, 4 mmol) in methanol (10 mL), hydroxylamine (50% solution in water) (0.86 mL, 14 mmol) was added at 25° C. and allowed to stir at 70° C. for overnight. The reaction mixture was concentrated under reduced pressure to obtain N′-hydroxy-2-(4-((phenylthio)methyl)phenyl)acetimidamide (1 g, 3.67 mmol, 92% yield).

### Step 4: Preparation of 3-(4-((phenylthio)methyl)benzyl)-5-(trifluoromethyl)-1,2,4-oxadiazole

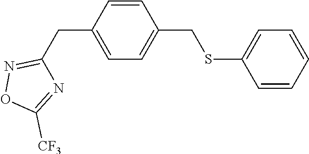 [SMILES: FC(F)(F)c1nc(Cc2ccc(CSc3ccccc3)cc2)no1]

To a solution of N′-hydroxy-2-(4-((phenylthio)methyl)phenyl)acetimidamide (0.9 g, 3.3 mmol) in tetrahydrofuran (10 mL), and trifluoroacetic anhydride (0.7 mL, 5 mmol) was added at 0° C. under nitrogen atmosphere. The reaction mixture was allowed to stir at 25° C. for 16 hours. The reaction mixture was added to the ice cold mixture of a solution of sodium bicarbonate (1.11 g, 13.2 mmol), and ethyl acetate (20 mL) and stirred for 5 min. The ethyl acetate layer was isolated, dried over sodium sulphate and concentrated under reduced pressure to obtain a crude product. The crude product was purified by column chromatography to obtain 3-(4-((phenylthio)methyl)benzyl)-5-(trifluoromethyl)-1,2,4-oxadiazole (0.79 g, 2.25 mmol, 68% yield).

1H-NMR (400 MHz, CHLOROFORM-D) δ 7.32-7.15 (m, 9H), 4.13 (s, 2H), 4.09 (s, 2H); LCMS (M+H): 350.2

### Example 13: Preparation of 3-(4-((phenylsulfinyl)methyl)benzyl)-5-(trifluoromethyl)-1,2,4-oxadiazole (Compound No. 105)

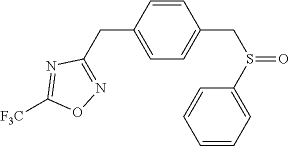 [SMILES: O=S(Cc1ccc(Cc2noc(C(F)(F)F)n2)cc1)c1ccccc1]

To a stirred solution of 3-(4-((phenylthio)methyl)benzyl)-5-(trifluoromethyl)-1,2,4-oxadiazole (0.4 g, 1.14 mmol) in dichloromethane (10 mL), m-chloroperbenzoic acid (0.296 g, 1.7 mmol) was added at 0° C. and allowed to stir at 25° C. for overnight. The reaction mixture was diluted with dichloromethane (10 mL), washed twice with aqueous sodium bicarbonate solution (10 mL) and concentrated under reduced pressure. The obtained crude product was purified by column chromatography on silica gel to get pure 3-(4-((phenylsulfinyl)methyl)benzyl)-5-(trifluoromethyl)-1,2,4-oxadiazole (0.1 g, 0.27 mmol, 24% yield).

1H-NMR (400 MHz, DMSO-D6) δ 7.53-7.49 (m, 5H), 7.24 (d, 2H), 7.07 (d, 2H), 4.25 (d, 3H), 4.02 (d, 1H); LCMS (M+H): 366.85

### Example 14: Preparation of 3-(4-((phenylsulfonyl)methyl)benzyl)-5-(trifluoromethyl)-1,2,4-oxadiazole (Compound No. 106)

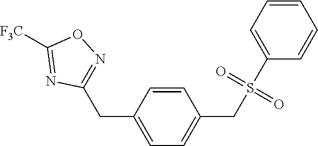 [SMILES: O=S(=O)(Cc1ccc(Cc2noc(C(F)(F)F)n2)cc1)c1ccccc1]

To a solution of 3-(4-((phenylthio)methyl)benzyl)-5-(trifluoromethyl)-1,2,4-oxadiazole (0.16 g, 0.46 mmol) in dichloromethane (10 mL), m-chloroperbenzoic acid (0.24 g, 1.37 mmol) was added at 25° C. and allowed to stir for 16 h. The reaction mixture was diluted with dichloromethane (10 mL) and the mixture was washed twice with aqueous sodium bicarbonate solution (10 mL). The dichloromethane layer was concentrated under reduced pressure to obtain 3-(4-((phenylsulfonyl)methyl)benzyl)-5-(trifluoromethyl)-1,2,4-oxadiazole (0.125 g, 0.33 mmol, 71% yield).

1H-NMR (400 MHz, DMSO-D6) δ 7.72-7.69 (m, 3H), 7.59-7.55 (m, 2H), 7.26-7.24 (d, 2H), 7.14-7.11 (d, 2H), 4.64 (s, 3H), 4.24 (s, 2H); LCMS (M−H): 381.05

As described herein the compounds of general Formula I show an extremely high fungicidal activity which is exerted with respect to numerous phytopathogenic fungi which attack on important agricultural crops. Compounds of present invention were assessed for activity against one or more of the following:

### BIOLOGY EXAMPLES

### Biological Test Examples (In Vitro Test)

### Example 1: Pyricularia oryzae (Rice Blast)

Compounds were dissolved in 0.3% DMSO and then added to Potato Dextrose Agar medium just prior to dispensing it into petri dishes. 5 mL medium with a compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds 14 16 21 40 52 showed >70% at 300 ppm control in these tests when compared to the untreated check which showed extensive disease development.

### Example 2: Alternaria solani (Early Blight of Tomato/Potato)

Compounds were dissolved in 0.3% DMSO and then added to Potato Dextrose Agar medium just prior to dispensing it into petri dishes. 5 mL medium with a compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds

| | | | | | | | | |
|---|---|---|---|---|---|---|---|---|
| 6 | 12 | 15 | 16 | 17 | 19 | 21 | 55 | 64 |

showed >70% at 300 ppm control in these tests when compared to the
untreated check which showed extensive disease development.

### Example 3: Colletotrichum capsici (Anthracnose)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compound 6 showed >70% at 300 ppm control in these tests when compared to the untreated check which showed extensive disease development.

### Example 4: Corynespora cassicola (Leaf Spot of Tomato)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 70% relative humidity for seven days and radial growth was measured. Compound 68 showed >70% at 300 ppm control in these tests when compared to the untreated check which showed extensive disease development

### Biological Test Examples (Greenhouse)

### Example A: Phakopsora pachyrhizi Test in Soybean

Compounds were dissolved in 2% DMSO/Acetone and then diluted with water containing emulsifier to the desired test concentration.

To test the preventive activity of compounds, healthy young soybean plants, raised in the greenhouse, were sprayed with the active compound solution at the stated application rates inside spray cabinets using hallowcone nozzles. One day after treatment, the plants were inoculated with a suspension containing 2.1×10⁶ Phakopsora pachyrhizi spores. The inoculated plants were then kept in the greenhouse chamber at 25° C. temperature and 90% relative humidity for disease expression.

A visual assessment of the compound's performance was carried out by rating the disease severity (0-100% scale) on treated plants on 3, 7, 10 and 15 days after application. Efficacy (% control) of the compounds was calculated by comparing the disease rating in the treatment with the one of the untreated control. The treated plants were also assessed for plant compatibility by recording symptoms like necrosis, chlorosis and stunting.

Compounds

| | | | | | | | | | | | |
|---|---|---|---|---|---|---|---|---|---|---|---|
| 2 | 3 | 4 | 5 | 6 | 8 | 9 | 10 | 12 | 13 | 18 | 27 |
| 29 | 30 | 38 | 40 | 42 | 45 | 46 | 47 | 48 | 49 | 50 | 67 |
| 68 | | | | | | | | | | | |

showed >70% at 500 ppm control in these tests when compared to the
untreated check which showed extensive disease development.

1. A compound of Formula I, 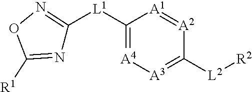
wherein,
R¹ is selected from the group consisting of C₁-C₂-monohaloalkyl, C₁-C₂-dihaloalkyl, C₁-C₂-trihaloalkyl, C₁-C₂-tetrahaloalkyl, and C₁-C₂-pentahaloalkyl;
A¹ is CR^{A1} or N;
A² is CR^{A2} or N;
A³ is CR^{A3} or N; &
A⁴ is CR^{A4} or N; wherein no more than two of A¹, A², A³ & A⁴ are nitrogen;
wherein, R^{A1}, R^{A2}, R^{A3}, R^{A4} and R^{A5} are independently and optionally selected from the group consisting of hydrogen, halogen, cyano, nitro, amino, hydroxy, C₁-C₆-alkyl, C₃-C₆-cycloalkyl, C₁-C₆-haloalkyl, C₁-C₆-hydroxyalkyl, C₁-C₆-alkoxy, C₁-C₆-alkoxy-C₁-C₆-alkyl, and C₁-C₆-haloalkoxy;
either R^{A1} and R^{A2} or R^{A3} and R^{A4} or both R^{A1} and R^{A2} as well as R^{A3} and R^{A4} together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S(O)₀₋₂; wherein, the carbocyclic or the heterocyclic ring or ring system may optionally further be substituted with one or more of halogen, C₁-C₆-alkyl, C₃-C₆-cycloalkyl, C₁-C₆-haloalkyl, C₁-C₆-hydroxyalkyl, C₁-C₆-alkoxy and C₁-C₆-haloalkoxy; L¹ is —C(R⁴R⁵)— or —C(═W)—;
L² is a direct bond, —C(R^{4a}R^{5a})—, —(NR⁶)₀₋₁C(═W¹)—(NR⁶)₀₋₁, —C(F₂)—, —C(R^{4a}R^{5a})C(═O)—, —O—, —(CR^{4a}R^{5a})₀₋₂S(═O)₀₋₂—, —N(R⁶)—, —(CR^{4a}R^{5a})₀₋₂C(═W¹)NR⁶(CR^{4a}R^{5a})₀₋₂—, and —NR⁶S(═O)₀₋₂—;
wherein W and W¹ is O or S;
wherein, R² is selected from the group consisting of hydrogen, halogen, cyano, nitro, amino, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, aryloxy, heteroaryloxy, C₃-C₈-heterocylyloxy, C₃-C₈-cycloalkyloxy, C₁-C₆-haloalkoxy, C₁-C₆-haloalkoxycarbonyl, C₁-C₆-alkylthio, arylthio, heteroarylthio, C₄-C₅-heterocyclylthio, C₁-C₆-haloalkylthio, C₁-C₆-haloalkylsulfinyl, C₁-C₆-haloalkylsulfonyl, arylsulfonyl, heteroarylsulfonyl, C₃-C₈-cycloalkylsulfonyl C₁-C₆-alkylsulfinyl, C₁-C₆-alkylsulfonyl, C₁-C₆-alkylamino, arylamino, heteroarylamino, C₄-C₈-heterocyclylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₃-C₆-cycloalkoxycarbonyl, aryloxycarbonyl, heteroaryloxycarbonyl, heterocycloxycarbonyl, C₁-C₆-alkylaminocarbonyl, C₁-C₆-dialkylaminocarbonyl, arylaminocarbonyl, heteroarylaminocarbonyl, C₃-C₆-cycloalkylaminocarbonyl, C₁-C₆-alkylaminocarbonylamino, C₁-C₆-dialkylaminocarbonylamino, arylaminocarbonylamino, heteroarylaminocarbonylamino, C₃-C₆-cycloalkylaminocarbonylamino, C₁-C₆-alkylcarbonylamino, C₃-C₆-cycloalkylcarbonylamino, arylcarbonylamino, heteroarylcarbonylamino, heterocyclylcarbonylamino, C₁-C₆-haloalkylcarbonylamino, C₁-C₆-alkyloxycarbonylamino, aryloxycarbonylamino, heterocycloxycarbonylamino, heteroaryloxycarbonylamino, C₃-C₆-cycloalkyloxycarbonylamino, C₁-C₆-alkoxycarbonyloxy, C₁-C₆-alkylaminocarbonyloxy, or C₁-C₆-dialkylaminocarbonyloxy, sulfilimines, sulfoximines, sulfonamide, and sulfinamide; R² may optionally further be substituted with one or more R⁷; or
R² is phenyl, benzyl, naphthyl, a 5- or 6-membered aromatic ring, an 8- to 11-membered aromatic multi-cyclic ring system, an 8- to 11-membered aromatic fused ring system, a 5- or 6-membered heteroaromatic ring, an 8- to 11-membered heteroaromatic multi-cyclic ring system or an 8- to 11-membered heteroaromatic fused ring system; wherein the heteroatom of the heteroaromatic ring or ring system is one or more heteroatom selected from N, O or S, and each phenyl, benzyl, aromatic or heteroaromatic ring or ring system may be optionally substituted with one or more substituents selected from R³; or
or R² and R⁶ together with the atoms to which they are attached form a 4-, 5-, 6- or 7-membered nonaromatic heterocyclic ring, an 8- to 15-membered nonaromatic hetero-multicyclic ring system, an 5- to 15 membered hetero-spirocyclic ring system, or an 8- to 15-membered nonaromatic heterocyclic fused ring system, wherein the heteroatom of the nonaromatic heterocyclic ring or ring system is selected from N, O or S(O)₀₋₂; and the C ring member of the nonaromatic heterocyclic ring or ring system may be replaced with C(═O), C(═S), C(═CR^{4b}R^{5b}) or C(═NR^{6a}) and each or nonaromatic heterocyclic ring or ring system may be optionally substituted with one or more substituents selected from R³;
wherein, R³ is independently selected from halogen, cyano, nitro, hydroxy, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₃-C₈-cycloalkyl-C₁-C₆-alkyl, C₃-C₈-cycloalkyl-C₃-C₈-cycloalkyl, C₃-C₈-cycloalkenyl, C₁-C₆-alkoxy-C₁-C₆-alkyl, C₃-C₈-cycloalkoxy-C₁-C₆-alkyl, C₁-C₆-alkylsulfinyl-C₁-C₆-alkyl, C₁-C₆-alkylsulfonyl-C₁-C₆-alkyl, C₁-C₆-alkylamino, di-C₁-C₆-alkylamino, C₁-C₆-alkylamino-C₁-C₆-alkyl, di-C₁-C₆-alkylamino-C₁-C₆-alkyl, C₁-C₆-haloalkylamino-C₁-C₆-alkyl, C₃-C₈-cycloalkylamino, C₃-C₈-cycloalkylamino-C₁-C₆-alkyl, C₁-C₆-alkylcarbonyl, C₁-C₆-haloalkoxy-C₁-C₆-alkyl, C₁-C₆-hydroxyalkyl, C₂-C₆-hydroxyalkenyl, C₂-C₆-hydroxyalkynyl, C₂-C₆-alkenyloxy, C₂-C₆-haloalkenyloxy, C₂-C₆-alkynyloxy, C₁-C₆-alkylcarbonylalkoxy, C₁-C₆-alkylthio, C₁-C₆-haloalkylthio, C₃-C₈-cycloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkylsulfonyl, C₃-C₈-cycloalkylsulfinyl, C₁-C₆-alkylsulfonylamino, C₁-C₆-haloalkylsulfonylamino, C₁-C₆-alkylsulfonyloxy, C₆-C₁₀-arylsulfonyloxy, C₆-C₁₀-arylsulfonyl, C₆-C₁₀-arylsulfinyl, C₆-C₁₀-arylthio, C₁-C₆-cyanoalkyl, C₁-C₆-haloalkylamino, C₁-C₆-alkoxyamino, C₁-C₆-haloalkoxyamino, C₁-C₆-alkoxycarbonylamino, C₁-C₆-alkylcarbonyl-C₁-C₆-alkylamino, C₂-C₆-alkenylthio, di(C₁-C₆-haloalkyl)amino-C₁-C₆-alkyl, C₁-C₆-alkylaminocarbonylamino, di(C₁-C₆-haloalkyl)amino, sulfilimines, sulfoximines or SF5; wherein, R³ may be optionally substituted with halogen, cyano, amino, C₁-C₆-alkyl, C₁-C₆-alkoxy, C₁-C₆-alkylamino-C₁-C₆-alkoxy, C₁-C₆-alkylthio, and C₃-C₈-cycloalkyl; or
R⁷ is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₃-C₈-cycloalkyloxy, aryloxy, C₁-C₆-haloalkoxy, and C₁-C₆-haloalkoxycarbonyl; or
two R⁷ together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S; or
R⁷ is phenyl, benzyl, a 5-membered aromatic ring, a 5- or 6-membered heteroaromatic ring; wherein heteroatom of the heteroaromatic ring is selected from N, O or S; or
R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N, O or S(O)₀₋₂; and the C ring member of the nonaromatic carbocyclic or nonaromatic heterocyclic ring may be replaced with C(═O), C(═S), C(═CR^{4c}R^{5c}) or C(═NR^{6b}),
wherein, R⁷ may be further substituted with one or more R^{4d} on C atom and with one or more R^{6c} on N atom;
R⁴, R^{4a}, R^{4b}, R^{4c}, R^{4d}, R⁵, R^{5a}, R^{5b}, and R^{5c} are independently selected from hydrogen, halogen, cyano, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₃-C₈-cycloalkyloxy, C₁-C₄-alkoxy, and C₁-C₄-haloalkoxy; or
all or either of R⁴ and R⁵; R^{4a} and R^{5a}; R^{4b} and R^{5b}; and R^{4c} and R^{5c}; together with the atoms to which they are attached may form a C₃-C₆ non-aromatic carbocylic ring or C₃-C₆ non-aromatic heterocylic ring;
R⁶, R^{6a}, R^{6b}, and R^{6c} are independently selected from the group consisting of hydrogen, cyano, hydroxy, NR^{b}R^{c}, (C═O)—R^{d}, (C═O)(C═O)—R^{d}, S(O)₀₋₂R^{e}, C₁-C₆-alkyl, C₁-C₆-haloalkyl, C₁-C₆-alkoxy, C₁-C₆-haloalkoxy, C₁-C₆-alkylamino, di-C₁-C₆-alkylamino, tri-C₁-C₆-alkylamino, and C₃-C₈-cycloalkyl;
R^{b} and R^{c} represent hydrogen, hydroxyl, cyano, C₁-C₄-alkyl, C₁-C₄-haloalkyl, C₁-C₄-alkoxy, C₃-C₈-cycloalkyl, aryl, heteroaryl, C₄-C₆-heterocyclyl, and C₃-C₈-halocycloalkyl;
R^{d} represents hydrogen, hydroxy, halogen, NR^{b}R^{c}, C₁-C₆-alkyl, C₁-C₆-haloalkyl, C₁-C₆-alkoxy, C₁-C₆-haloalkoxy, C₃-C₈-cycloalkyl aryloxy, heteroaryloxy, C₃-C₈-cycloalkoxy, and C₃-C₈-halocycloalkyl; and
R^{e} represents hydrogen, halogen, cyano, C₁-C₆-alkyl, C₁-C₆-haloalkyl, C₁-C₆-alkoxy, C₁-C₆-haloalkoxy, C₃-C₈-cycloalkyl, and C₃-C₈-halocycloalkyl;
or N-oxides, metal complexes, isomers, polymorphs or the agriculturally acceptable salts thereof,
provided the following compounds are excluded from the definition of Formula I:
N-[4-[[5-(trifluoromethyl)-1,2,4-oxadiazol-3-yl]methyl]phenyl]-methanesulfonamide [Cas No. 1128079-05-9],
N-[4-[[5-(trifluoromethyl)-1,2,4-oxadiazol-3-yl]methyl]phenyl]-acetamide [Cas No. 943828-64-6],
1,2,4-Oxadiazole, 3-[(2,6-dichloro-4-hydrazinylphenyl)methyl]-5-(trifluoromethyl) [Cas No. 164157-03-3], and
4-[[5-(trifluoromethyl)-1,2,4-oxadiazol-3-yl]methyl]-benzoic acid methyl ester [Cas No. 2368917-79-5].
1,2,4-Oxadiazole, 3-[(4-methoxyphenyl)methyl]-5-(trifluoromethyl) [Cas No. 2322048-55-3],

2. The compound of claim 1, wherein
R¹ is C₁-C₂-dihaloalkyl or C₁-C₂-trihaloalkyl;
A¹ is CR^{A1} or N;
A² is CR^{A2} or N;
A³ is CR^{A3} or N; &
A⁴ is CR^{A4} or N; wherein no more than one of A¹, A², A³ & A⁴ are nitrogen;
wherein, R^{A1}, R^{A2}, R^{A3}, R^{A4} and R^{A5} are independently and optionally selected from the group consisting of hydrogen, halogen, cyano, C₁-C₆-alkyl, C₃-C₆-cycloalkyl, C₁-C₆-haloalkyl, and C₁-C₆-alkoxy;
L¹ is —C(R⁴R⁵)— or —C(═W)—;
L² is —(NR⁶)₀₋₁C(═W¹)—(NR⁶)₀₋₁, —(CR^{4a}R^{5a})₁₋₂S(═O)₀₋₂—, —(CR^{4a}R^{5a})₀₋₂C(═W¹)NR⁶(CR^{4a}R^{5a})₀₋₂, and NR⁶—NR⁶S(═O)₀₋₂—;
wherein W and W¹ is O or S;
wherein, R² is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, aryloxy, heteroaryloxy, C₃-C₈-heterocylyloxy, C₃-C₈-cycloalkyloxy, C₁-C₆-alkylthio, arylthio, heteroarylthio, C₄-C₈-heterocyclylamino, C₁-C₆-dialkylamino, and C₃-C₈-cycloalkylamino; or
R² is phenyl, benzyl, a 5- or 6-membered heteroaromatic ring; wherein the heteroatom of the heteroaromatic ring is one or more heteroatom selected from N, O or S, and each phenyl, benzyl or heteroaromatic ring may be optionally substituted with one or more substituents selected from R³; or
or R² and R⁶ together with the atoms to which they are attached form a 4-, 5- or 6-membered nonaromatic heterocyclic ring, wherein the heteroatom of the nonaromatic heterocyclic ring is selected from N or O; and nonaromatic heterocyclic ring may be optionally substituted with one or more substituents selected from R³;
wherein, R³ is independently selected from halogen, cyano, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₁-C₆-alkylamino, di-C₁-C₆-alkylamino, and C₁-C₆-alkoxy; or
R⁴, R^{4a}, R^{4b}, R⁵, R^{5a} and R^{5b} are independently selected from hydrogen, halogen, C₁-C₄-alkyl, C₁-C₄-haloalkyl, C₂-C₄-haloalkenyl, C₃-C₆-cycloalkyl, C₃-C₆-halocycloalkyl, C₃-C₈-cycloalkyloxy, C₁-C₄-alkoxy, and C₁-C₄-haloalkoxy; or
all or either of R⁴ and R⁵; R^{4a} and R^{5a}; and R^{4b} and R^{5b}; together with the atoms to which they are attached may form a C₃-C₆ non-aromatic carbocylic ring or C₃-C₆ non-aromatic heterocylic ring;
R⁶ and R^{6a} are independently selected from the group consisting of hydrogen, S(O)₀₋₂R^{e}, C₁-C₆-alkyl, C₁-C₆-haloalkyl, C₁-C₆-alkoxy, C₁-C₆-haloalkoxy, and C₃-C₈-cycloalkyl;
R^{e} represents hydrogen, C₁-C₆-alkyl, C₁-C₆-haloalkyl, C₁-C₆-alkoxy, C₁-C₆-haloalkoxy, C₃-C₈-cycloalkyl, and C₃-C₈-halocycloalkyl;
or N-oxides, metal complexes, isomers, polymorphs or the agriculturally acceptable salts thereof.

3. The compound of claim 1, wherein
R¹ is C₁-C₂-trihaloalkyl;
A¹ is CH;
A² is CH;
A³ is CH; &
A⁴ is CH;
L¹ is —C(R⁴R⁵)— or —C(═W)—;
L² is —(NR⁶)₀₋₁C(═W¹)—(NR⁶)₀₋₁, —(CR^{4a}R^{5a})₁₋₂S(═O)₀₋₂—, —(CR^{4a}R^{5a})₀₋₂C(═W¹)NR⁶(CR^{4a}R^{5a})₀₋₂, and NR⁶—NR⁶S(═O)₀₋₂—;
wherein W and W¹ is O or S;
wherein, R² is selected from the group consisting of C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-alkoxy, aryloxy, heteroaryloxy, C₃-C₈-heterocylyloxy, C₄-C₈-heterocyclylamino, and C₁-C₆-dialkylamino; or
R² is phenyl, benzyl, a 5- or 6-membered heteroaromatic ring; wherein the heteroatom of the heteroaromatic ring is one or more heteroatom selected from N, O or S, and each phenyl, benzyl or heteroaromatic ring may be optionally substituted with one or more substituents selected from R³; or
or R² and R⁶ together with the atoms to which they are attached form a 4-, 5- or 6-membered nonaromatic heterocyclic ring, wherein the heteroatom of the nonaromatic heterocyclic ring is selected from N or O; and nonaromatic heterocyclic ring may be optionally substituted with one or more substituents selected from R³;
wherein, R³ is independently selected from halogen, cyano, C₁-C₆-alkyl, C₁-C₆-haloalkyl, C₃-C₈-cycloalkyl, C₁-C₆-alkylamino, di-C₁-C₆-alkylamino, and C₁-C₆-alkoxy; or
R⁴, R^{4a}, R^{4b}, R⁵, R^{5a} and R^{5b} are independently selected from hydrogen, halogen, C₁-C₄-alkyl, C₁-C₄-haloalkyl, C₃-C₆-cycloalkyl, C₃-C₆-halocycloalkyl, C₃-C₈-cycloalkyloxy, C₁-C₄-alkoxy and C₁-C₄-haloalkoxy; or
all or either of R⁴ and R⁵; R^{4a} and R^{5a}; and R^{4b} and R^{5b}; together with the atoms to which they are attached may form a C₃-C₆ non-aromatic carbocylic ring;
R⁶ and R^{6a} are independently selected from the group consisting of hydrogen, C₁-C₆-alkyl, C₁-C₆-haloalkyl, and C₃-C₈-cycloalkyl;
or N-oxides, metal complexes, isomers, polymorphs or the agriculturally acceptable salts thereof.

4. The compound of claim 1, wherein the compound is selected from the group consisting of:
4-methyl-N-(4-((5-(trifluoromethyl)-1,2,4-oxadiazol-3-yl)methyl)phenyl)benzamide; N-(4-((5-(trifluoromethyl)-1,2,4-oxadiazol-3-yl)methyl)phenyl)benzamide; N-(4-((5-(trifluoromethyl)-1,2,4-oxadiazol-3-yl)methyl)phenyl)picolinamide; N-(4-((5-(trifluoromethyl)-1,2,4-oxadiazol-3-yl)methyl)phenyl)nicotinamide; N-(4-((5-(trifluoromethyl)-1,2,4-oxadiazol-3-yl)methyl)phenyl)isonicotinamide; 2-phenyl-N-(4-((5-(trifluoromethyl)-1,2,4-oxadiazol-3-yl)methyl)phenyl)acetamide; 4-cyano-N-(4-((5-(trifluoromethyl)-1,2,4-oxadiazol-3-yl)methyl)phenyl)benzamide; 4-(trifluoromethyl)-N-(4-((5-(trifluoromethyl)-1,2,4-oxadiazol-3-yl)methyl)phenyl)benzamide; 4-fluoro-N-(4-((5-(trifluoromethyl)-1,2,4-oxadiazol-3-yl)methyl)phenyl)benzamide; 4-chloro-N-(4-((5-(trifluoromethyl)-1,2,4-oxadiazol-3-yl)methyl)phenyl)benzamide; 2-(4-fluorophenyl)-N-(4-((5-(trifluoromethyl)-1,2,4-oxadiazol-3-yl)methyl)phenyl)acetamide; N-(4-fluorobenzyl)-4-((5-(trifluoromethyl)-1,2,4-oxadiazol-3-yl)methyl)benzamide; morpholino(4-((5-(trifluoromethyl)-1,2,4-oxadiazol-3-yl)methyl)phenyl)methanone; N-(3-fluorobenzyl)-4-((5-(trifluoromethyl)-1,2,4-oxadiazol-3-yl)methyl)benzamide; N-(1-(p-tolyl)ethyl)-4-((5-(trifluoromethyl)-1,2,4-oxadiazol-3-yl)methyl)benzamide; N-(pyridin-3-ylmethyl)-4-((5-(trifluoromethyl)-1,2,4-oxadiazol-3-yl)methyl)benzamide; N-(5-chloropyridin-3-yl)-4-((5-(trifluoromethyl)-1,2,4-oxadiazol-3-yl)methyl)benzamide; N-(2-chloro-5-methoxyphenyl)-4-((5-(trifluoromethyl)-1,2,4-oxadiazol-3-yl)methyl)benzamide; N-(2-methoxyphenyl)-4-((5-(trifluoromethyl)-1,2,4-oxadiazol-3-yl)methyl)benzamide; N-(4-methoxyphenyl)-4-((5-(trifluoromethyl)-1,2,4-oxadiazol-3-yl)methyl)benzamide; N-(2-morpholinoethyl)-4-((5-(trifluoromethyl)-1,2,4-oxadiazol-3-yl)methyl)benzamide; N-(4-chlorophenyl)-4-((5-(trifluoromethyl)-1,2,4-oxadiazol-3-yl)methyl)benzamide; N-(3-fluorobenzyl)-N-methyl-4-((5-(trifluoromethyl)-1,2,4-oxadiazol-3-yl)methyl)benzamide; N-(isoxazol-3-yl)-4-((5-(trifluoromethyl)-1,2,4-oxadiazol-3-yl)methyl)benzamide; 4-methoxy-N-(4-(5-(trifluoromethyl)-1,2,4-oxadiazole-3-carbonyl)phenyl)benzamide; 4-chloro-N-(4-(5-(trifluoromethyl)-1,2,4-oxadiazole-3-carbonyl)phenyl)benzamide; N-(4-(5-(trifluoromethyl)-1,2,4-oxadiazole-3-carbonyl)phenyl)isonicotinamide; N-(4-(5-(trifluoromethyl)-1,2,4-oxadiazole-3-carbonyl)phenyl)nicotinamide; tert-butyl (4-(5-(trifluoromethyl)-1,2,4-oxadiazole-3-carbonyl)phenyl)carbamate; tert-butyl (4-(difluoro(5-(trifluoromethyl)-1,2,4-oxadiazol-3-yl)methyl)phenyl)carbamate; 2-(4-fluorophenyl)-N-(4-(5-(trifluoromethyl)-1,2,4-oxadiazole-3-carbonyl)phenyl)acetamide; N-(4-(difluoro(5-(trifluoromethyl)-1,2,4-oxadiazol-3-yl)methyl)phenyl)-4-(trifluoromethyl)benzamide; N-(4-(difluoro(5-(trifluoromethyl)-1,2,4-oxadiazol-3-yl)methyl)phenyl)-2-phenylacetamide; N-(4-(difluoro(5-(trifluoromethyl)-1,2,4-oxadiazol-3-yl)methyl)phenyl)-4-fluorobenzamide; N-(4-(difluoro(5-(trifluoromethyl)-1,2,4-oxadiazol-3-yl)methyl)phenyl)benzamide; N-(4-(difluoro(5-(trifluoromethyl)-1,2,4-oxadiazol-3-yl)methyl)phenyl)-2-(4-fluorophenyl)acetamide; 4-cyano-N-(4-(difluoro(5-(trifluoromethyl)-1,2,4-oxadiazol-3-yl)methyl)phenyl)benzamide; N-(4-(difluoro(5-(trifluoromethyl)-1,2,4-oxadiazol-3-yl)methyl)phenyl)-4-methylbenzamide; N-(4-(difluoro(5-(trifluoromethyl)-1,2,4-oxadiazol-3-yl)methyl)phenyl)picolinamide; N-methyl-4-((5-(trifluoromethyl)-1,2,4-oxadiazol-3-yl)methyl)benzamide; N,N-dimethyl-4-((5-(trifluoromethyl)-1,2,4-oxadiazol-3-yl)methyl)benzamide; N-(2-methoxyethyl)-4-((5-(trifluoromethyl)-1,2,4-oxadiazol-3-yl)methyl)benzamide; N-allyl-4-((5-(trifluoromethyl)-1,2,4-oxadiazol-3-yl)methyl)benzamide; azetidin-1-yl(4-((5-(trifluoromethyl)-1,2,4-oxadiazol-3-yl)methyl)phenyl)methanone; pyrrolidin-1-yl(4-((5-(trifluoromethyl)-1,2,4-oxadiazol-3-yl)methyl)phenyl)methanone; N-(2-methoxyethyl)-N-methyl-4-((5-(trifluoromethyl)-1,2,4-oxadiazol-3-yl)methyl)benzamide; N-(cyclopropylmethyl)-4-((5-(trifluoromethyl)-1,2,4-oxadiazol-3-yl)methyl)benzamide; N-ethyl-N-methyl-4-((5-(trifluoromethyl)-1,2,4-oxadiazol-3-yl)methyl)benzamide; N-aIlyl-N-methyl-4-((5-(trifluoromethyl)-1,2,4-oxadiazol-3-yl)methyl)benzamide; N-(prop-2-yn-1-yl)-4-((5-(trifluoromethyl)-1,2,4-oxadiazol-3-yl)methyl)benzamide; N-phenyl-4-((5-(trifluoromethyl)-1,2,4-oxadiazol-3-yl)methyl)benzamide; tert-butyl (4-((5-(trifluoromethyl)-1,2,4-oxadiazol-3-yl)methyl)phenyl)carbamate; N-(3,4-dichlorophenyl)-4-((5-(trifluoromethyl)-1,2,4-oxadiazol-3-yl)methyl)benzamide; N-(p-tolyl)-4-((5-(trifluoromethyl)-1,2,4-oxadiazol-3-yl)methyl)benzamide; N-(3-chlorophenyl)-4-((5-(trifluoromethyl)-1,2,4-oxadiazol-3-yl)methyl)benzamide; N-(4-(dimethylamino)phenyl)-4-((5-(trifluoromethyl)-1,2,4-oxadiazol-3-yl)methyl)benzamide; N-(4-(tert-butyl)phenyl)-4-((5-(trifluoromethyl)-1,2,4-oxadiazol-3-yl)methyl)benzamide; N-(m-tolyl)-4-((5-(trifluoromethyl)-1,2,4-oxadiazol-3-yl)methyl)benzamide; 4-((5-(trifluoromethyl)-1,2,4-oxadiazol-3-yl)methyl)-N-(3-(trifluoromethyl)phenyl)benzamide; N-(3-fluorophenyl)-4-((5-(trifluoromethyl)-1,2,4-oxadiazol-3-yl)methyl)benzamide; N-(2-fluorophenyl)-4-((5-(trifluoromethyl)-1,2,4-oxadiazol-3-yl)methyl)benzamide; N-(4-fluorophenyl)-4-((5-(trifluoromethyl)-1,2,4-oxadiazol-3-yl)methyl)benzamide; N-(2,4-dichlorophenyl)-4-((5-(trifluoromethyl)-1,2,4-oxadiazol-3-yl)methyl)benzamide; N-(m-tolyl)-4-((5-(trifluoromethyl)-1,2,4-oxadiazol-3-yl)methyl)benzothioamide; N-(4-(dimethylamino)phenyl)-4-((5-(trifluoromethyl)-1,2,4-oxadiazol-3-yl)methyl)benzothioamide; N-(3-fluorophenyl)-4-((5-(trifluoromethyl)-1,2,4-oxadiazol-3-yl)methyl)benzothioamide; N-(4-fluorophenyl)-4-((5-(trifluoromethyl)-1,2,4-oxadiazol-3-yl)methyl)benzothioamide; N-(4-((5-(trifluoromethyl)-1,2,4-oxadiazol-3-yl)methyl)phenyl)benzenesulfonamide; 4-fluoro-N-(4-((5-(trifluoromethyl)-1,2,4-oxadiazol-3-yl)methyl)phenyl)benzenesulfonamide; 4-methyl-N-(4-((5-(trifluoromethyl)-1,2,4-oxadiazol-3-yl)methyl)phenyl)benzenesulfonamide; N-(3-fluorobenzyl)-4-((5-(trifluoromethyl)-1,2,4-oxadiazol-3-yl)methyl)benzothioamide; 3-chloro-N-(4-((5-(trifluoromethyl)-1,2,4-oxadiazol-3-yl)methyl)phenyl)benzenesulfonamide; 1-isopropyl-3-(4-((5-(trifluoromethyl)-1,2,4-oxadiazol-3-yl)methyl)phenyl)urea; 1-(pyridin-3-yl)-3-(4-((5-(trifluoromethyl)-1,2,4-oxadiazol-3-yl)methyl)phenyl)urea; 1-(4-methoxyphenyl)-3-(4-((5-(trifluoromethyl)-1,2,4-oxadiazol-3-yl)methyl)phenyl)urea; 1-(p-tolyl)-3-(4-((5-(trifluoromethyl)-1,2,4-oxadiazol-3-yl)methyl)phenyl)urea; 1-(4-chlorophenyl)-3-(4-((5-(trifluoromethyl)-1,2,4-oxadiazol-3-yl)methyl)phenyl)urea; 1-(4-fluorophenyl)-3-(4-((5-(trifluoromethyl)-1,2,4-oxadiazol-3-yl)methyl)phenyl)urea; 2-fluoro-N-(4-((5-(trifluoromethyl)-1,2,4-oxadiazol-3-yl)methyl)phenyl)benzenesulfonamide; 1-phenyl-3-(4-((5-(trifluoromethyl)-1,2,4-oxadiazol-3-yl)methyl)phenyl)urea; 1-ethyl-3-(4-((5-(trifluoromethyl)-1,2,4-oxadiazol-3-yl)methyl)phenyl)urea; N-phenyl-4-(2-(5-(trifluoromethyl)-1,2,4-oxadiazol-3-yl)propan-2-yl)benzamide; N-(p-tolyl)-4-(2-(5-(trifluoromethyl)-1,2,4-oxadiazol-3-yl)propan-2-yl)benzamide; N-(4-chlorophenyl)-4-(2-(5-(trifluoromethyl)-1,2,4-oxadiazol-3-yl)propan-2-yl)benzamide; N-(pyridin-4-yl)-4-(2-(5-(trifluoromethyl)-1,2,4-oxadiazol-3-yl)propan-2-yl)benzamide; 3-(trifluoromethyl)-N-(4-((5-(trifluoromethyl)-1,2,4-oxadiazol-3-yl)methyl)phenyl)benzenesulfonamide; N-(2-methoxyphenyl)-4-(2-(5-(trifluoromethyl)-1,2,4-oxadiazol-3-yl)propan-2-yl)benzamide; N-(pyridin-3-yl)-4-(2-(5-(trifluoromethyl)-1,2,4-oxadiazol-3-yl)propan-2-yl)benzamide; 1-(cyclopropylmethyl)-3-(4-((5-(trifluoromethyl)-1,2,4-oxadiazol-3-yl)methyl)phenyl)urea; 1-(tert-butyl)-3-(4-((5-(trifluoromethyl)-1,2,4-oxadiazol-3-yl)methyl)phenyl)urea; phenyl (4-((5-(trifluoromethyl)-1,2,4-oxadiazol-3-yl)methyl)phenyl)carbamate; methyl (4-((5-(trifluoromethyl)-1,2,4-oxadiazol-3-yl)methyl)phenyl)carbamate; N-(4-((5-(trifluoromethyl)-1,2,4-oxadiazol-3-yl)methyl)benzyl)cyclopropanecarboxamide; 4-methyl-N-(4-((5-(trifluoromethyl)-1,2,4-oxadiazol-3-yl)methyl)benzyl)benzamide; 2-fluoro-N-(4-((5-(trifluoromethyl)-1,2,4-oxadiazol-3-yl)methyl)benzyl)benzamide; 3-fluoro-N-(4-((5-(trifluoromethyl)-1,2,4-oxadiazol-3-yl)methyl)benzyl)benzamide; 3-chloro-N-(4-((5-(trifluoromethyl)-1,2,4-oxadiazol-3-yl)methyl)benzyl)benzamide; N-(4-((5-(trifluoromethyl)-1,2,4-oxadiazol-3-yl)methyl)benzyl)propionamide; N-phenyl-4-(1-(5-(trifluoromethyl)-1,2,4-oxadiazol-3-yl)cyclopropyl)benzamide; N-(p-tolyl)-4-(1-(5-(trifluoromethyl)-1,2,4-oxadiazol-3-yl)cyclopropyl)benzamide; N-(4-chlorophenyl)-4-(1-(5-(trifluoromethyl)-1,2,4-oxadiazol-3-yl)cyclopropyl)benzamide; N-(2-methoxyphenyl)-4-(1-(5-(trifluoromethyl)-1,2,4-oxadiazol-3-yl)cyclopropyl)benzamide; 3-(4-((phenylthio)methyl)benzyl)-5-(trifluoromethyl)-1,2,4-oxadiazole; 3-(4-((phenylsulfinyl)methyl)benzyl)-5-(trifluoromethyl)-1,2,4-oxadiazole; and 3-(4-((phenylsulfonyl)methyl)benzyl)-5-(trifluoromethyl)-1,2,4-oxadiazole.

5. A composition comprising the compound of claim 1 in a combination with at least one further pesticidally active substance selected from the group consisting of fungicides, insecticides, nematicides, acaricides, biopesticides, herbicides, safeners, plant growth regulators, antibiotics, fertilizers and nutrients.

6. A composition comprising the compound of claim 1 and at least one agrochemically acceptable auxiliary.

7. The composition of claim 6, wherein the composition further comprises at least one additional active ingredient.

8. A composition comprising the compound of claim 1 applied to seed, wherein the amount of the compound of the formula I is from 0.1 gai to 10 kgai per 100 kg of seeds.

9. A method for controlling or preventing phytopathogenic fungi, wherein the method comprises treating the fungi or the materials, plants, plant parts, locus thereof, soil or seeds to be protected against fungal attack, with an effective amount of the compound of claim 1.

10. A method for controlling or preventing infestation of plants by phytopathogenic micro-organisms in agricultural crops and or horticultural crops wherein an effective amount of at least one compound of claim 1, is applied to the seeds of plants.

11. The compound of claim 1, for controlling or preventing plant diseases.

12. The compound of claim 1, used as a fungicides.

13. The method of claim 9, wherein the plant diseases are rust pathogens selected from the group consisting of Puccinia spp. (rusts), comprising P. triticina (brown or leaf rust), P. striiformis (stripe or yellow rust), P. hordei (dwarf rust), P. graminis (stem or black rust) and P. recondita (brown or leaf rust) on cereals viz., wheat, barley or rye, P. melanocephala on sugarcane and Phakopsora spp. comprising Phakopsora pachyrhizi and P. meibomiae on soybeans, Hemileia vastatrix (Coffee rust), Uromyces spp., comprising U. fabae (rust of beans).

14. A process for preparing in the compound of claim 1, said process comprising any of the steps of:
a. reacting a nitrile derivative of Formula (i) with hydroxylamine salt in the presence of a suitable base to obtain hydroxyl imidamide derivative of Formula (ii), 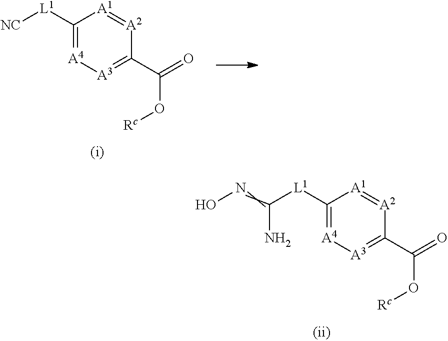
wherein, R^{c} is C₁-C₄-alkyl; L¹ is CR⁴R⁵;
b. reacting the hydroxyl imidamide derivative of Formula (ii) with an anhydride of Formula (V-a) or an acid halide of Formula (V-b) to obtain a compound of Formula (iii), 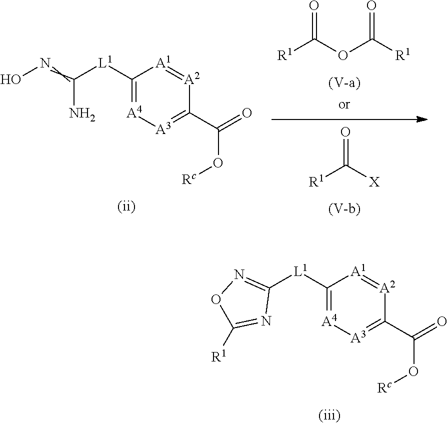
wherein, R^{c} is C₁-C₄-alkyl; L¹ is CR⁴R⁵; X is halide;
c. reacting the compound of Formula (iii) with an amine in the presence of trialkyl aluminium to obtain the compound of Formula I, 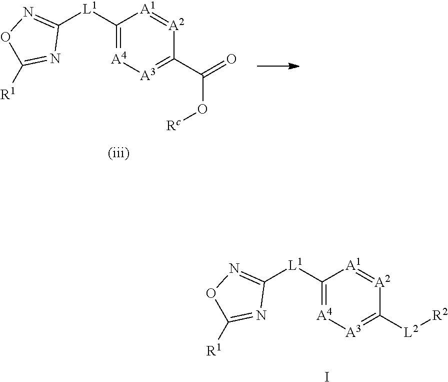
wherein, R^{c} is C₁-C₄-alkyl; L¹ is CR⁴R⁵; L² is C(═O)NR⁶;
d. reacting a nitrile derivative of Formula (iv) with hydroxylamine salt in the presence of a suitable base to obtain hydroxyl imidamide derivative of Formula (v), 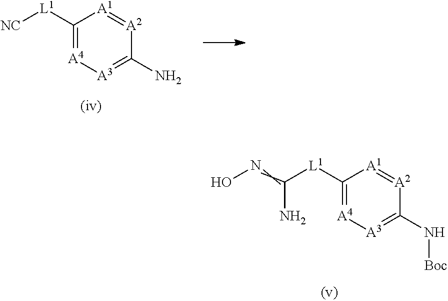
wherein, L¹ is CR⁴R⁵, C(═W) or CF₂;
e. reacting the hydroxyl imidamide derivative of Formula (v) with an anhydride of Formula (V-a) or an acid halide of Formula (V-b) to obtain a compound of Formula (vi), 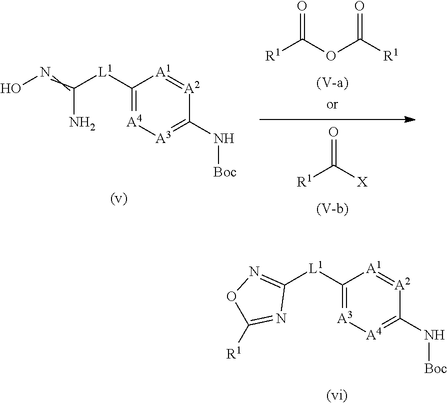
wherein, L¹ is CR⁴R⁵, C(═W) and CF₂; X is halide;
f. converting the compound of Formula (vi) into the compound of Formula (vii) using a suitable reagent, 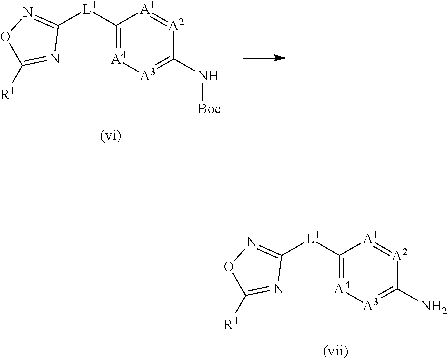
wherein, L¹ is CR⁴R⁵, C(═W) and CF₂;
g. reacting the compound of Formula (vii) with a suitable reactant to obtain the compound of Formula I, 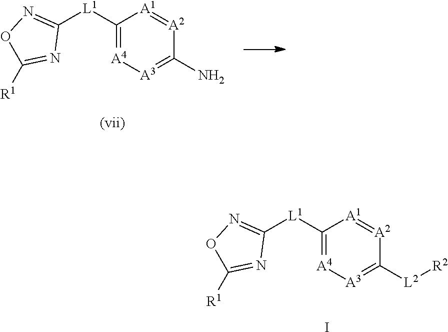
wherein, the suitable reactant is acid or acid halide when L² is NR⁶C(═O); L¹ is CR⁴R⁵, C(═W) and CF₂; and the reaction is carried out using a suitable base optionally in the presence of a suitable coupling reagent;
the suitable reactant is sulphonyl chloride when L² is NR⁶; R² is selected from the group consisting of C₁-C₆-haloalkylsulfonyl, arylsulfonyl, heteroarylsulfonyl, C₃-C₈-cycloalkylsulfonyl C₁-C₆-alkylsulfinyl, and C₁-C₆-alkylsulfonyl; L¹ is CR⁴R⁵, C(═W) and CF₂; and the reaction is carried out using a suitable base;
the suitable reactant is hydroxy compound when L² is NR⁶C(═O); R² is C₁-C₆-alkoxy, aryloxy, heteroaryloxy, C₃-C₈-heterocylyloxy, C₃-C₈-cycloalkyloxy, and C₁-C₆-haloalkoxy; L¹ is CR⁴R⁵, C(═W) and CF₂; and the reaction is carried out using a suitable reagent;
the suitable reactant is amine compound when L² is NR⁶C(═O); R² is C₁-C₆-alkylamino, arylamino, heteroarylamino, C₄-C₈-heterocyclylamino, C₁-C₆-dialkylamino, C₃-C₈-cycloalkylamino, and C₁-C₆-alkyl-C₃-C₈-cycloalkylamino; L¹ is CR⁴R⁵, C(═W) and CF₂; and the reaction is carried out using a suitable reagent;
h. fluorinating a compound of Formula (d) to obtain the compound of Formula (vi) using a suitable fluorinating agent, 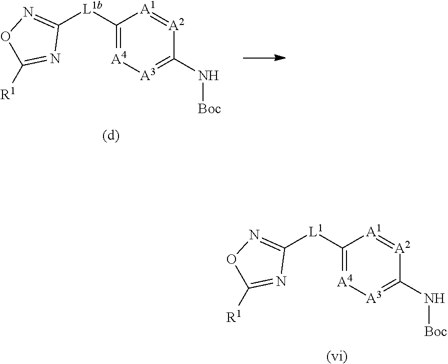
wherein, L¹ is CF₂; L^{1b} is C(═O);
i. reacting a nitrile derivative of Formula (g) with hydroxylamine salt in the presence of a suitable base to obtain hydroxyl imidamide derivative of Formula (h), 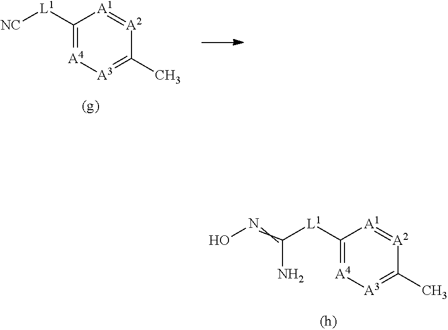
wherein, L¹ is CR⁴R⁵;
j. reacting the hydroxyl imidamide derivative of Formula (h) with an anhydride of Formula (V-a) or an acid halide of Formula (V-b) to obtain a compound of Formula (i), 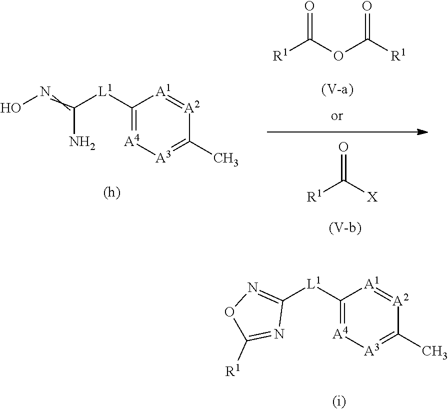
wherein, L¹ is CR⁴R⁵; X is halide;
k. brominating the compound of Formula (i) using a suitable brominating reagent in the presence of a suitable radical initiator to obtain a compound of Formula (j), 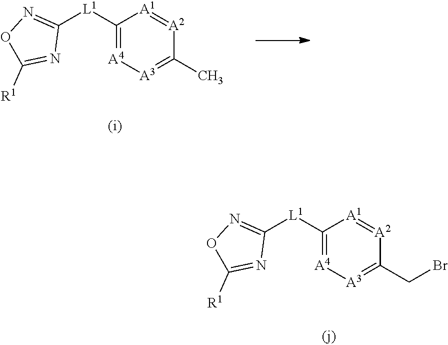
wherein, L¹ is CR⁴R⁵;
l. converting the compound of Formula (j) into a compound of Formula (k) using a suitable metal azide, 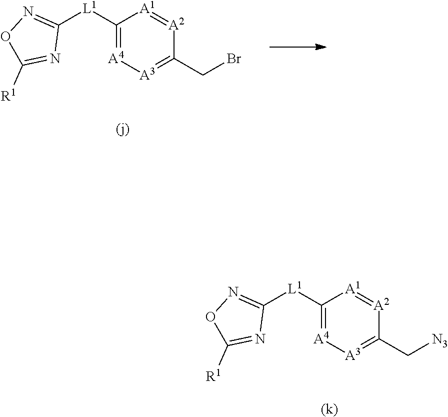
wherein, L¹ is CR⁴R⁵;
m. reducing the compound of Formula (k) into a compound of Formula (l) using a suitable phosphine reagent, 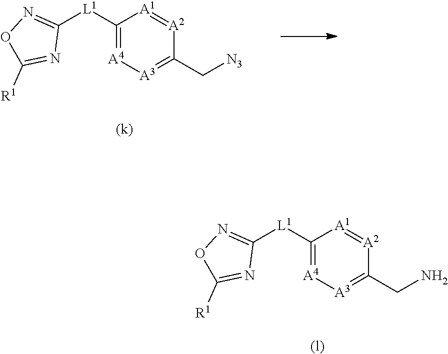
wherein, L¹ is CR⁴R⁵;
n. reacting the compound of Formula (I) with a suitable reactant to obtain the compound of Formula I, 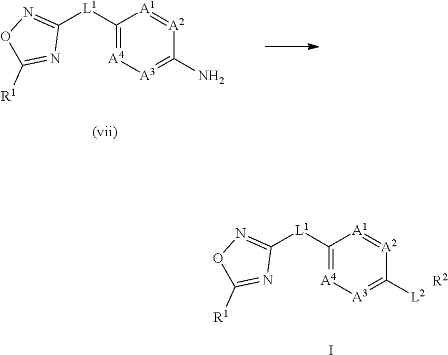
wherein, the suitable reactant is acid or acid halide when L² is CR⁴R⁵; R² is selected from the group consisting of C₁-C₆-alkylcarbonylamino, C₃-C₆-cycloalkylcarbonylamino, arylcarbonylamino, heteroarylcarbonylamino, heterocyclylcarbonylamino, and C₁-C₆-haloalkylcarbonylamino, L¹ is CR⁴R⁵; and the reaction is carried out using a suitable base optionally in the presence of a suitable coupling reagent;
the suitable reactant is sulphonyl chloride when L² is CR⁴R⁵; R² is sulfonamide; L¹ is CR⁴R⁵; and the reaction is carried out using a suitable base;
the suitable reactant is isocyanate compound when L² is CR⁴R⁵; and R² is selected from the group consisting of C₁-C₆-alkylaminocarbonylamino, C₁-C₆-dialkylaminocarbonylamino, arylaminocarbonylamino, heteroarylaminocarbonylamino, and C₃-C₆-cycloalkylaminocarbonylamino; L¹ is CR⁴R⁵;
the suitable reactant is chloroformate compound when L² is CR⁴R⁵; and R² is selected from the group consisting of C₁-C₆-alkyloxycarbonylamino, aryloxycarbonylamino, heterocycloxycarbonylamino, heteroaryloxycarbonylamino, and C₃-C₆-cycloalkyloxycarbonylamino; L¹ is CR⁴R⁵; and the reaction is carried out using a suitable reagent;
o. brominating the compound of Formula (g) using a suitable brominating reagent in the presence of a suitable radical initiator to obtain a compound of Formula (m), 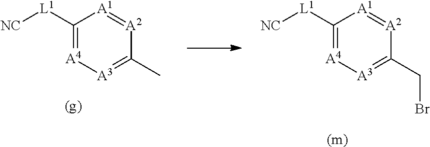
wherein, L¹ is CR⁴R⁵;
p. reacting the compound of Formula (m) with a mercapto compound in the presence of a suitable base, 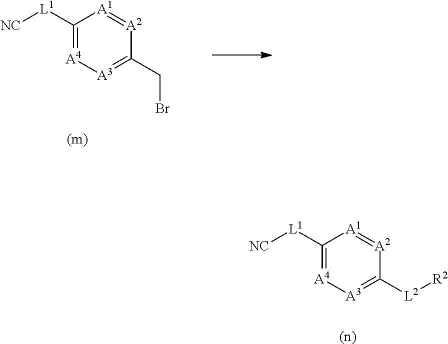
wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio;
q. reacting a nitrile derivative of Formula (n) with hydroxylamine salt in the presence of a suitable base to obtain hydroxyl imidamide derivative of Formula (o), 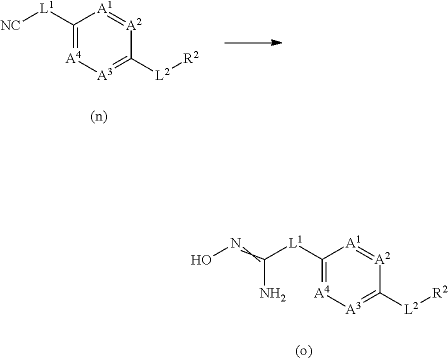
wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio;
r. reacting the hydroxyl imidamide derivative of Formula (o) with an anhydride of Formula (V-a) or an acid halide of Formula (V-b) to obtain the compound of Formula I, 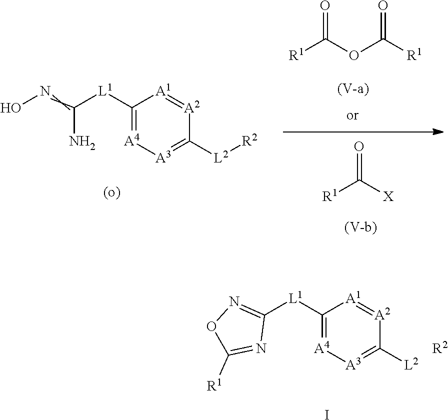
wherein, L¹ is CR⁴R⁵; L² is CH₂; R² is selected from the group consisting of C₁-C₆-alkylthio, arylthio, heteroarylthio, C₄-C₅-heterocyclylthio, and C₁-C₆-haloalkylthio; X is halide;
s. oxidizing a compound of Formula (p) to obtain the compound of Formula I using a suitable oxidizing reagent, 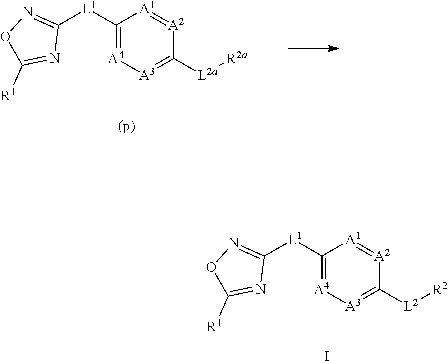
wherein, L¹ is CR⁴R⁵; L² is CH₂; L^{2a} is CH₂; R^{2a} is selected from the group consisting of C₁-C₆-alkylthio, arylthio, heteroarylthio, C₄-C₅-heterocyclylthio, and C₁-C₆-haloalkylthio; R² is selected from the group consisting of C₁-C₆-haloalkylsulfinyl, arylsulfinyl, heteroarylsulfinyl, C₃-C₈-cycloalkylsulfinyl, C₁-C₆-alkylsulfinyl, C₁-C₆-haloalkylsulfonyl, arylsulfonyl, heteroarylsulfonyl, C₃-C₈-cycloalkylsulfonyl, and C₁-C₆-alkylsulfonyl;
t. hydrolyzing an ester of Formula (q) into an acid of Formula (r) using suitable hydrolyzing agent, 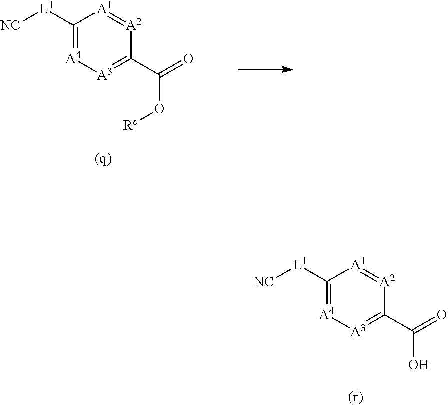
wherein, R^{c} is C₁-C₄-alkyl; L¹ is CR⁴R⁵;
u. the acid of Formula (r) is reacted with hydroxylamine salt in the presence of a suitable base to obtain the compound of Formula (s), 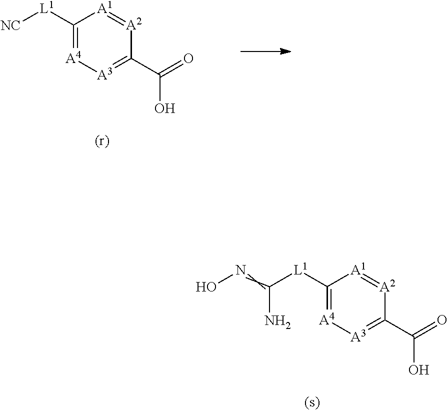
wherein, L¹ is CR⁴R⁵;
v. reacting the compound of Formula (s) with an anhydride of Formula (V-a) or an acid halide of Formula (V-b) to obtain the compound of Formula (t), 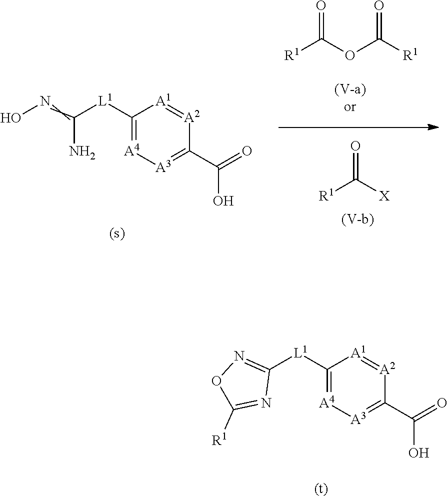
wherein, L¹ is CR⁴R⁵; X is halide;
w. reacting the compound of Formula (t) with an amine NHR⁶R² in the presence of a suitable coupling reagent and a suitable base to obtain the compound of Formula I, 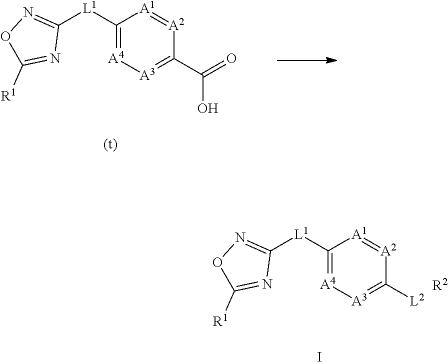
wherein, L¹ is CR⁴R⁵; and
x. converting a compound of Formula (u) into the compound of Formula I using a Lawesson's reagent, 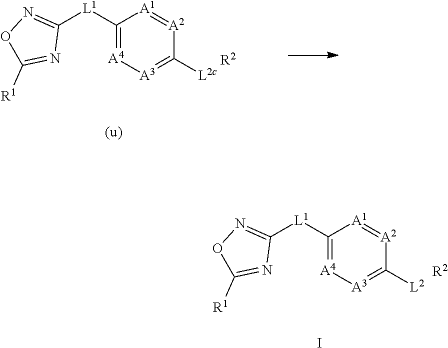
wherein, L¹ is CR⁴R⁵; L² is C(═S); R² is selected from the group consisting of C₁-C₆-alkylamino, arylamino, heteroarylamino, C₄-C₈-heterocyclylamino, C₁-C₆-dialkylamino, C₃-C₈-cycloalkylamino, and C₁-C₆-alkyl-C₃-C₈-cycloalkylamino; L^{2c} is C(═O).

15. A Compound of Formula (ii), 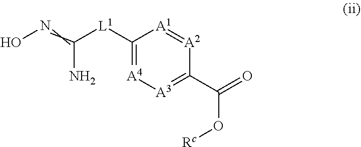
wherein, L¹ is —C(R⁴R⁵)— or —C(═W)—; R⁴ & R⁵ are as defined in claim 1 excluding hydrogen; R^{c} is C₁-C₄-alkyl; and A¹, A², A³, and A⁴ are as defined in claim 1.

16. A compound of Formula (vi), 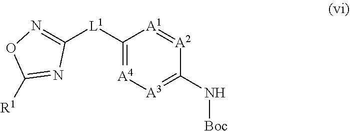
wherein, L¹ is —C(R⁴R⁵)— or —C(═W)—; R¹ is CF₃, CF₂Cl or CHF₂ and R⁴, R⁵ A¹, A², A³, and A⁴ are as defined in claim 1.

17. A compound of Formula (vii), 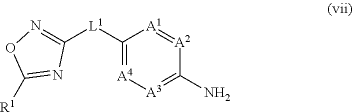
wherein, L¹ is —C(R⁴R⁵)— or —C(═W)—; R¹ is CF₃, CF₂Cl or CHF₂ and R⁴, R⁵ A¹, A², A³, and A⁴ are as defined in claim 1.

18. A compound of Formula (k), 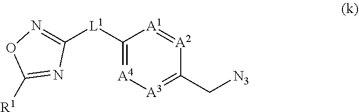
wherein, L¹ is —C(R⁴R⁵)— or —C(═W)—; R¹ is CF₃, CF₂Cl or CHF₂ and R⁴, R⁵ A¹, A², A³, and A⁴ are as defined in claim 1.

19. A compound of Formula (t), 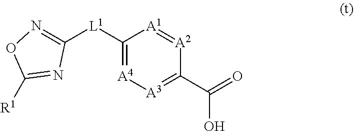
wherein, L¹ is —C(R⁴R⁵)— or —C(═W)—; R¹ is CF₃, CF₂Cl or CHF₂ and R⁴, R⁵ A¹, A², A³, and A⁴ are as defined in claim 1.
